SEQUENCE LISTING (39 sequences)

SEQ ID NO: 1 - protein, 7 aa, synthetic construct
NGSGQNQ

SEQ ID NO: 2 - protein, 4 aa, synthetic construct
AQAQ

SEQ ID NO: 3 - protein, 7 aa, synthetic construct
DGAATKN

SEQ ID NO: 4 - protein, 11 aa, synthetic construct
DGTLAVPFKAQ

SEQ ID NO: 5 - protein, 7 aa, synthetic construct
DGQSSKS

SEQ ID NO: 6 - protein, 11 aa, synthetic construct
AQPHEGSSRAQ

SEQ ID NO: 7 - protein, 11 aa, synthetic construct
AQPNASVNSAQ

SEQ ID NO: 8 - protein, 11 aa, synthetic construct
AQLNTTKPIAQ

SEQ ID NO: 9 - protein, 11 aa, synthetic construct
AQGNNTRSVAQ

SEQ ID NO: 10 - protein, 11 aa, synthetic construct
AQNNSTRGGAQ

SEQ ID NO: 11 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIDGAATKNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIDGQSSKSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPHEGSSRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPNASVNSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLNTTKPIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIDGAATKNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQNNSTRGGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 766 aa, synthetic construct
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 20 - protein, 350 aa, synthetic construct
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 21 - protein, 166 aa, synthetic construct
MVLASSTTSIHTMLLLLLMLFHLGLQASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVEC

SEQ ID NO: 22 - protein, 143 aa, synthetic construct
MSRLPVLLLLHLLVSPGLQAPMTQTTSLKTSWAKCSNMIDEIITHLNQPPLPSPDFNNLNEEDQTILVEKNLRRSNLEAFSKAVKSLQNASAIESILKNLPPCLPMATAAPTRPPIRITNGDRNDFRRKLKFYLKTLENEQAQ

SEQ ID NO: 23 - protein, 152 aa, synthetic construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 24 - protein, 555 aa, synthetic construct
MMDNKDLEAEIHPLKNEDKKSQENPGNLPRNEDNLKSKPVPSRLSRCRTVAFFLSLFTCLFVVFVLSFIIPCPDRPSSQGTWKLDYNNAVMYDFLALGDINKDKVQDVLFLYKNTNSSNNLTRSCADEGFSTPCAFVVAVSGANGSVLWERPVAQDVALVKCAMPQTLDSDEVSSACIVVGRAGSFVAVSFFTGETLWSHPSSFSGNVSILSPLLQVPDIDGDGDGTPDLLILAQEGQEVSGALYSGSTGYQIGHRGSLGVDGDGVALLHVTRTGAQYILLPCASALCGFSVKSLYERITGRDGHFKEDPYWENMLNHSVHRRLLHRLGAVRYLMNIPGKAGQDLLLVTSEACVLLDGQDLEPRWTLGEVQVLRKPILGHYKPDTLAVVIENGTSIDRQILLLDLSTGSILWSQPLPSLPGGPPSTSLMTADHRSAFFFWGLHDLVSTNEMDPPDVQHSLYMFHPTLPGILLELANVSANIVAFDAVLLEPSRHAAYVLLTGPASSDVPGLVSVTKHKVQDLVPGSRVIHLGEGSSDSDQAIRDRFSRLRYRSEM

SEQ ID NO: 25 - protein, 555 aa, synthetic construct; identical to SEQ ID NO: 24

SEQ ID NO: 26 - protein, 534 aa, synthetic construct
MKRMVGCDLLWLAAASCVLTLVSPSTIHQGYGRPRNSSNLDLDCGSPDSPSSGICFDPCQNHTVLNDPTRSTENNDSSVAWCDDNLHGWYRFVGDGGVKMPETCVSVFRCHTSAPMWLSGSHPILGDGIVSHTACANWNENCCFWRSEVQVKACSEELGEYHVYKLQGTPECSLRYCTDPSTAPKNCEITCRPEEECVFQNNNWSCVCRQDLHVSDSQSLQPLLDCGDNEIKVKLDKCLLGGMGFKEEIIAYLNDRNCNGTMQDEPNNWVSMTSPVVANYCGNILEKNGTHAIYRNTLSLATDFIIRDFRVNVNFQCAYPLDMSVSLETALQPIVSSLTVDVDGAGEFNVKMALFQDQSYTNPYEGAEVLLPVESILYVGVLLNRGDTSRFKLLLTNCYATPSEDRHDPVKYFIIKNRCPNQRDSTINVRENGVSSESRFSVQMFMFAGNYDLVFLHCEVYLCDSTTEQCQPSCSTNRLRSSRPAIDYNRVLDLGPITKRSAQSSATSKGTPHTTGFLLAWPMFFLPVFLALLF

SEQ ID NO: 27 - protein, 534 aa, synthetic construct
MPHLMERIVGSGLLWLALVSCILAQASAVQQGYGNPSEASSYGLDLDCGAPGTPEAHICFDPCQNYTLLDEPFRSTENSEEKEGCDDNMSGWYRFAGEGGVRMSETCVQVHRCQTAAPMWLNGTHPALGDGIVNRTACAHWSGNCCLWKTEVLVKACPGGYHVYRLEGTPECSLRYCTDPSTVEDKCEKACRPEEECLARNSTWGCFCRQDLNSSDFHSLQPQLDCGAEEIKVKVDKCLLGGLGLEEEVIAYLRDPNCSSILQTGETNWVSVTSPVQASACRNILERNQTHAIYKNTLSLVSDFIIRDTILNINFQCAYPLDMKVSLQAALKPIVSFLNVSVGGDGEFIVRMALFQDQNYTNPYEGDVAELSVESMLYVGAILEQGDTSRFNLVLRNCYATPTEDKADPVKYFIIRNSCSNQRDSTIHVEENGRSSESRFSVQMFMFAGHYDLVFLHCEIHLCDSFNEQCQPSCPRSQVRSEVPAIDLSRVLDLGPITRRGAQSPGVMNGTPGTAGFLVAWPMVLLTVLLAWLF

SEQ ID NO: 28 - protein, 537 aa, synthetic construct
MPHLMERMVGSGLLWLALVSCILTQASAVQRGYGNPIEASSYGLDLDCGAPGTPEAHVCFDPCQNYTLLDEPFRSTENSAGSQGCDKNMSGWYRFVGEGGVRMSETCVQVHRCQTDAPMWLNGTHPALGDGITNHTACAHWSGNCCFWKTEVLVKACPGGYHVYRLEGTPWCNLRYCTVPRDPSTVEDKCEKACRPEEECLALNSTWGCFCRQDLNSSDVHSLQPQLDCGPREIKVKVDKCLLGGLGLGEEVIAYLRDPNCSSILQTEERNWVSVTSPVQASACRNILERNQTHAIYKNTLSLVNDFIIRDTILNINFQCAYPLDMKVSLQAALQPIVSSLNVSVDGNGEFIVRMALFQDQNYTNPYEGDAVELSVESVLYVGAILEQGDTSRFNLVLRNCYATPTEDKADLVKYFIIRNSCSNQRDSTIHVEENGQSSESRFSVQMFMFAGHYDLVFLHCEIHLCDSLNEQCQPSCSRSQVRSEVPAIDLARVLDLGPITRRGAQSPGVMNGTPSTAGFLVAWPMVLLTVLLAWLF

SEQ ID NO: 29 - protein, 1613 aa, synthetic construct
MGAVLRSLLACSFCVLLRAAPLLLYANRRDLRLVDATNGKENATIVVGGLEDAAAVDFVFGHGLIYWSDVSEEAIKRTEFNKSESVQNVVVSGLLSPDGLACDWLGEKLYWTDSETNRIEVSNLDGSLRKVLFWQELDQPRAIALDPSSGFMYWTDWGEVPKIERAGMDGSSRFVIINTEIYWPNGLTLDYQERKLYWADAKLNFIHKSNLDGTNRQAVVKGSLPHPFALTLFEDTLYWTDWNTHSILACNKYTGEGLREIHSNIFSPMDIHAFSQQRQPNATNPCGIDNGGCSHLCLMSPVKPFYQCACPTGVKLMENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGTIYWGDAKTDKIEVMNTDGTGRRVLVEDKIPHIFGFTLLGDYVYWTDWQRRSIERVHKRSAEREVIIDQLPDLMGLKATSVHRVIGSNPCAEDNGGCSHLCLYRPQGLRCACPIGFELIGDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSDMLGLNREVIADDLPHPFGLTQYQDYIYWTDWSRRSIERANKTSGQNRTIIQGHLDYVMDILVFHSSRQAGWNECASSNGHCSHLCLAVPVGGFVCGCPAHYSLNADNRTCSAPSTFLLFSQKSAINRMVIDEQQSPDIILPIHSLRNVRAIDYDPLDKQLYWIDSRQNSIRKAHEDGGQGFNVVANSVANQNLEIQPYDLSIDIYSRYIYWTCEATNVIDVTRLDGRSVGVVLKGEQDRPRAIVVNPEKGYMYFTNLQERSPKIERAALDGTEREVLFFSGLSKPIALALDSKLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGDIDCIPVAWRCDGFTECEDHSDELNCPVCSESQFQCASGQCIDGALRCNGDANCQDKSDEKNCEVLCLIDQFRCANGQCVGKHKKCDHSVDCSDRSDELDCYPTEEPAPQATNTVGSVIGVIVTIFVSGTIYFICQRMLCPRMKGDGETMTNDYVVHSPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDVNYDSEPVPPPPTPRSQYLSAEENYESCPPSPYTERSYSHHLYPPPPSPCTDSS

SEQ ID NO: 30 - protein, 1613 aa, synthetic construct
MGAVLRSLLACSFCVLLRAAPLLLYANRRDLRLVDATNGKENATIVVGGLEDAAAVDFVFGHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSNIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYALNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSIGVVLKGEQDRPRAIVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTIYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 31 - protein, 1613 aa, synthetic construct
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAVVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 32 - protein, 967 aa, synthetic construct
MAKGFYISKSLGILGILLGVAAVCTIIALSVVYSQEKNKNANSSPVASTTPSASATTNPASATTLDQSKAWNRYRLPNTLKPDSYRVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 33 - protein, 144 aa, synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 34 - protein, 322 aa, synthetic construct
MKTLAGLVLGLVIFDAAVTAPTLESINYDSETYDATLEDLDNLYNYENIPVDKVEIEIATVMPSGNRELLTPPPQPEKAQEEEEEEESTPRLIDGSSPQEPEFTGVLGPHTNEDFPTCLLCTCISTTVYCDDHELDAIPPLPKNTAYFYSRFNRIKKINKNDFASLSDLKRIDLTSNLISEIDEDAFRKLPQLRELVLRDNKIRQLPELPTTLTFIDISNNRLGRKGIKQEAFKDMYDLHHLYLTDNNLDHIPLPLPENLRALHLQNNNILEMHEDTFCNVKNLTYIRKALEDIRLDGNPINLSKTPQAYMCLPRLPVGSLV

SEQ ID NO: 35 - protein, 15 aa, synthetic construct
AQGNNTRSVAQAQTG

SEQ ID NO: 36 - protein, 15 aa, synthetic construct
AQLNTTKPIAQAQTG

SEQ ID NO: 37 - protein, 17 aa, synthetic construct
DNVDADKVMITNEEEIK

SEQ ID NO: 38 - protein, 16 aa, synthetic construct
AVKSLQNASAIESILK

SEQ ID NO: 39 - protein, 27 aa, synthetic construct
TINGSGQNQQTLKFSVAGPSNMAVQGR

(19) United States
(12) Patent Application Publication
(10) Pub. No.: US 2024/0139329 A1
(43) Pub. Date: May 2, 2024

(54) Targets for Receptor-Mediated Control of Therapeutic Biodistribution and Efficacy

(71) Applicant: California Institute of Technology, Pasadena, CA (US)

(72) Inventors: Viviana Gradinaru, La Canada Flintridge, CA (US); Timothy F. Shay, Pasadena, CA (US); Xinhong Chen, Pasadena, CA (US); Seongmin Jang, Pasadena, CA (US)

(21) Appl. No.: 18/495,508

(22) Filed: Oct. 26, 2023

Related U.S. Application Data
(60) Provisional application No. 63/420,002, filed on Oct. 27, 2022.
(60) Provisional application No. 63/465,814, filed on May 11, 2023.

(51) Int. Cl. A61K 47/64; A61K 48/00; C07K 14/00

(52) U.S. Cl. CPC A61K 47/64; A61K 48/0041; C07K 14/001

(57) ABSTRACT

Disclosed herein include novel blood-brain barrier (BBB)-crossing receptors on the BBB interface, targeting peptides and derivatives thereof capable of binding to the novel receptors, and related methods of using the receptors to increase the permeability of the BBB and to deliver an agent to a nervous system (e.g., CNS). In some embodiments, the BBB-crossing receptor is LRP6. Disclosed herein also include recombinant adeno-associated viruses (rAAVs) with increased specificity and transduction efficiency across the BBB and related compositions and methods of treating various diseases and conditions.

### RELATED APPLICATIONS

This application claims the benefit under 35 U.S.C. § 119(e) of U.S. Provisional Patent Application Ser. No. 63/420,002, filed Oct. 27, 2022; and U.S. Provisional Patent Application Ser. No. 63/465,814, filed May 11, 2023. The contents of these related applications are incorporated herein by reference in their entireties for all purposes.

### STATEMENT REGARDING FEDERALLY SPONSORED R&D

This invention was made with government support under Grant No. NS111369 awarded by National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365863-US_SequenceListing, created Oct. 23, 2023, which is 51,181 bytes in size. The information in the electronic format of the Sequence Listing is incorporated herein by reference in its entirety.

### BACKGROUND

### Field

The present disclosure relates generally to the field of gene delivery. More specifically, methods and compositions are disclosed for crossing the blood brain barrier.

### Description of the Related Art

Targeting therapeutic and research molecules to tissues and cell types of interest and away from tissues and cell types mediating potentially hazardous side effects is a foundational problem for drug development. This is especially the case for molecules targeting the brain. The blood-brain barrier (BBB) sharply limits the properties of therapeutic and research molecules that may enter the central nervous system (CNS) upon systemic administration into the peripheral bloodstream.

There is a need for using a gain-of-function rather than loss-of-function screen, and screening both naturally- and lab-evolved AAVs. There is a need for methods to reverse engineer novel targets that determine therapeutic biodistribution the products of natural evolution and of directed evolution on adeno-associated viruses (AAVs).

### SUMMARY

Disclosed herein include methods of increasing permeability of the blood brain barrier. In some embodiments, the method comprises: providing a targeting peptide capable of binding to low density lipoprotein receptor related protein 6 (LRP6), thereby increasing permeability of the blood brain barrier.

In some embodiments, the targeting peptide binds YWTD domain 1 and/or domain 2 of LRP6. In some embodiments, the permeability of the blood brain barrier is increased by at least 25%, 50%, 75%, 100%, or more as compared to the absence of the targeting peptide.

Disclosed herein include methods of delivering a payload to a nervous system of a subject. In some embodiments, the method comprises: providing a targeting peptide capable of binding to low density lipoprotein receptor related protein 6 (LRP6) or a derivative thereof, wherein the targeting peptide is part of a delivery system, and wherein the delivery system comprises the payload to be delivered to the nervous system; and administering the delivery system to the subject.

In some embodiments, the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and any combination thereof. In some embodiments, the delivery system comprises a viral vector or a non-viral vector. In some embodiments, the targeting peptide enhances the binding affinity of the viral vector or the non-viral vector to LRP6. In some embodiments, the viral vector comprises an AAV vector. In some embodiments, the targeting peptide is part of a capsid protein of an AAV vector. In some embodiments, the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof. In some embodiments, the non-viral vector comprises lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

In some embodiments, the payload to be delivered to a nervous system is a biological molecule, a non-biological molecule, or a combination thereof. In some embodiments, the biological molecule is selected from the group consisting of a nucleic acid sequence, a protein, a peptide, a lipid, a polysaccharide, and any combination thereof. In some embodiments, the payload is a therapeutic molecule. In some embodiments, the nucleic acid sequence to be delivered to a nervous system comprises one or more of: a) a sequence encoding a trophic factor, a growth factor, or other soluble factors that might be released from the transduced cells and affect the survival or function of that cell and/or surrounding cells; b) a DNA that restores protein function to humans or animals harboring a genetic mutation(s) in that gene; c) a DNA that encodes a protein that can be used to control or alter the activity or state of a cell; d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

In some embodiments, the LRP6 is a mouse LRP6. In some embodiments, the LRP6 has an amino acid sequence having at least 80% sequence identity to an amino acid sequence of SEQ ID NO: 29. In some embodiments, the LRP6 is a macaque LRP6. In some embodiments, the LRP6 has an amino acid sequence having at least 80% sequence identity to an amino acid sequence of SEQ ID NO: 30. In some embodiments, the LRP6 is a human LRP6. In some embodiments, the LRP6 has an amino acid sequence having at least 80% sequence identity to an amino acid sequence of SEQ ID NO: 31. In some embodiments, upon binding the targeting peptide is capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

In some embodiments, the targeting peptide is inserted between two adjacent amino acids in AA587-594 of SEQ ID NO: 11 of the AAV9 vector or functional equivalents of AA587-594 in an amino acid sequence at least 80% identical to SEQ ID NO: 11. In some embodiments, the targeting peptide is inserted between AA588-589 of SEQ ID NO: 11 of the AAV9 vector or functional equivalents of AA588-589 in an amino acid sequence at least 80% identical to SEQ ID NO: 11.

The AAV vector can be conjugated to, e.g., a nanoparticle, a second molecule, or a combination thereof. The administration can be, e.g., a systemic administration. In some embodiments, the administration is an intravenous administration or an intrathecal administration.

In some embodiments, the subject is a mammal. In some embodiments, the subject is a human. In some embodiments, the subject is a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. In some embodiments, the lysosomal storage disorder that involve cells within the CNS is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease. In some embodiments, the subject is a subject suffering from, at risk to develop, or has suffered from a stroke, traumatic brain injury, epilepsy, or spinal cord injury.

Disclosed herein include adeno-associated virus (AAV) capsid proteins. In some embodiments, the AAV capsid protein comprises a targeting peptide having a binding specificity to LRP6. In some embodiments, the targeting peptide is part of a capsid protein of an rAAV vector. In some embodiments, the targeting peptide is inserted between two adjacent amino acids in AA587-594 of SEQ ID NO: 11 or functional equivalents of AA587-594 in an amino acid sequence at least 80% identical to SEQ ID NO: 11. In some embodiments, upon binding to LRP6 the targeting peptide is capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31. In some embodiments, the AAV is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof.

Disclosed herein include recombinant adeno-associated virus (rAAV). In some embodiments, the rAAV comprises any of the AAV capsid protein disclosed herein. In some embodiments, the rAAV comprises an AAV capsid protein which comprises a targeting peptide having a binding specificity to low density lipoprotein receptor related protein 6 (LRP6), wherein the amino acid sequence of the targeting peptide is inserted between two adjacent amino acids in AA587-594, or functional equivalents thereof, of the AAV capsid protein. In some embodiments, the two adjacent amino acids are AA588 and AA589. In some embodiments, upon binding to LRP6 the targeting peptide is capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, 5114, E115, R141, W157, W183, or W242 in LRP6 having an amino acid sequence of SEQ ID NO: 31. In some embodiments, the rAAV has enhanced tropism for the nervous system relative to an rAAV that does not comprise the targeting peptide. In some embodiments, the rAAV is capable of transducing the nervous system with an efficiency at least 2-fold higher than an rAAV that does not comprise the targeting peptide.

Disclosed herein include compositions for use in the delivery of an agent to a nervous system of a subject in need. In some embodiments, the composition comprises an AAV comprising (1) an AAV capsid protein disclosed herein and (2) an agent to be delivered to the nervous system of the subject; optionally wherein the nervous system is the central nervous system (CNS), the peripheral nervous system (PNS), or a combination thereof. In some embodiments, the nervous system is brain endothelial cells, neurons, capillaries in the brain, arterioles in the brain, arteries in the brain, or a combination thereof. In some embodiments, the composition is a pharmaceutical composition comprising one or more pharmaceutical acceptable carriers. In some embodiments, the agent to be delivered comprises a nucleic acid, a peptide, a small molecule, an aptamer, or a combination thereof.

Disclosed herein include antibodies or fragments thereof. In some embodiments, the antibody or fragment thereof comprises an amino acid sequence having a binding specificity to LRP6. In some embodiments, the antibody or fragment thereof is a bispecific antibody comprising at least one Fab having specificity to LRP6. Disclosed herein include antibody conjugates comprising any of the antibody or fragment thereof disclosed herein. In some embodiments, the antibody conjugate further comprises a therapeutic agent or a detectable label. Disclosed herein include peptides or a derivative or a conjugate thereof, having specificity to low density lipoprotein receptor related protein 6 (LRP6). Disclosed herein include nucleic acids. In some embodiments, the nucleic acid comprises a sequence encoding any of the antibody or fragment thereof or the peptide or any of the derivative or the conjugate thereof disclosed herein.

Disclosed herein include delivery systems. In some embodiments, the delivery system comprises (1) a targeting peptide having specificity to low density lipoprotein receptor related protein 6 (LRP6); and (2) an agent. In some embodiments, the targeting peptide is (1) displayed on the surface of the delivery system; or (2) partially embedded in the delivery system. In some embodiments, the delivery system is selected from the group consisting of: nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a viral vector or a non-viral vector. In some embodiments, the delivery system comprises a nanoparticle selected from the group consisting of: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

Disclosed herein include methods of designing a targeting peptide having specificity to low density lipoprotein receptor related protein 6 (LRP6). In some embodiments, the method comprises: generating in silico one or more targeting peptides each capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

In some embodiments, generating in silico the one or more targeting peptides comprises: generating in silico a plurality of candidate peptides; performing computer-assisted docking simulations for each of the plurality of candidate peptides binding to LRP6; and analyzing the structure of LRP6 binding to one or more of the plurality of candidate peptides to identify one or more targeting peptides capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

The method can comprise: obtaining a binding score for each of the plurality of candidate peptides binding to LRP6; and selecting one or more of the plurality of candidate peptides having a binding score above a threshold value as a targeting peptide having specificity to LRP6. The method can comprise: comparing the binding scores of two or more of the plurality of candidate peptides to rank the candidate peptide sequences. In some embodiments, obtaining the binding score for each of the plurality of candidate peptide sequences comprises (1) counting a total number of atoms in the interface of a candidate peptide and LRP6; (2) counting a total number of atoms in the candidate peptide, wherein the atoms are clashing with LRP6; (3) obtaining a binding angle of the candidate peptide; and (4) obtaining a binding depth of the candidate peptide.

Disclosed herein include agents capable of binding to a protein selected from the group consisting of interleukin 3 (IL3), Family With Sequence Similarity 234 Member A (FAM234A), Glycoprotein 2 (GP2), Dipeptidyl peptidase-4 (DPP4), Dickkopf WNT Signaling Pathway Inhibitor 3 (DKK3), Alanyl Aminopeptidase (ANPEP), Epiphycan (EPYC), and LRP6. In some embodiments, the agent is selected from the group consisting of an antibody or fragment thereof, an aptamer, a small molecule, a nucleic acid, and a peptide. In some embodiments, the antibody or fragment thereof comprises an Fc domain. In some embodiments, the antibody or fragment thereof is a single-chain variable fragment (scFv), a single-domain antibody, an immunoglobulin molecule, a monoclonal antibody, a chimeric antibody, a CDR-grafted antibody, a humanized antibody, a Fab fragment, a Fab′ fragment, a F(ab′)2 fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof. In some embodiments, the nucleic acid is a miRNA, an shRNA, and siRNA, or an oligonucleotide.

In some embodiments, the agent is conjugated to a detectable label. In some embodiments, the detectable label is selected from the group consisting of biotin, a fluorophore, a luminescent or bioluminescent marker, a radiolabel, an enzyme, an enzyme substrate, a quantum dot, an imaging agent, a metal particle, a magnetic particle, and any combination thereof. In some embodiments, the agent is a therapeutic agent. In some embodiments, the agent is a targeting peptide. In some embodiments, the targeting peptide is part of a delivery system wherein the delivery system comprises a payload to be delivered to a cell. In some embodiments, the delivery system comprises a viral or a non-viral vector. In some embodiments, the viral vector comprises an AAV vector. In some embodiments, the targeting peptide is part of a capsid protein of an AAV vector. In some embodiments, the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof. In some embodiments, the non-viral vector comprises lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

In some embodiments, the payload to be delivered to the cell is a biological molecule, a non-biological molecule, or a combination thereof. In some embodiments, the biological molecule is selected from the group consisting of a nucleic acid sequence, a protein, a peptide, a lipid, a polysaccharide, and any combination thereof. In some embodiments, the payload is a therapeutic molecule. In some embodiments, the nucleic acid sequence to be delivered to a nervous system comprises one or more of: a) a sequence encoding a trophic factor, a growth factor, or other soluble factors that might be released from the transduced cells and affect the survival or function of that cell and/or surrounding cells; b) a DNA that restores protein function to humans or animals harboring a genetic mutation(s) in that gene; c) a DNA that encodes a protein that can be used to control or alter the activity or state of a cell; d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 displays non-limiting exemplary data showing Surface Plasmon Resonance (SPR) confirms direct binding interaction between LRP6 extracellular domain and both AAV-X1.1 and AAV.CAP-Mac. No binding is detected with AAV9.

FIG. 2 displays exemplary computational modeling in AlphaFold2 of engineered AAV insertion peptides with human LRP6 extracellular domain shows binding to domain 2 for X1.1 and binding to either of domain 1 or domain 2 for CAP-Mac.

FIG. 3 displays exemplary data related to AAV-X1 and AAV.CAP-Mac show enhanced potency on HEK293 cells compared to standard AAV9-based AAVs that have not gained interactions with a human receptor.

FIG. 4 displays non-limiting exemplary data showing knockdown of endogenous LRP6 in HEK293 cells via siRNA selectively decreases the potency of AAV-X1.1 and AAV.CAP-Mac.

FIG. 5 displays non-limiting exemplary screening library and confirmation of results. Test AAVs and controls were added to fixed microarray slides.

FIG. 6 displays non-limiting exemplary screening library and confirmation of results. Test AAVs and controls were added to fixed microarray slides.

FIG. 7A-FIG. 7D display non-limiting exemplary data related to confirmation and specificity screens. Test AAVs were added to fixed confirmation slides using the direct fix method (AAVs not removed). Detection was performed using anti-AAV9 antibody followed by AF647 anti-mIgG H+L. Anti-Adeno-associated Virus 9, clone HL2372, supplied by Merck, Cat #MABF2309-100UL, 1:500 dilution. Detection antibody: AlexaFluor647 anti-mIgG H+L.

FIG. 8A-FIG. 8B display non-limiting exemplary data related to confirmation and specificity screens. Test and control AAVs were added to fixed confirmation slides using the direct fix method (AAVs not removed). Detection was performed using anti-AAV9 antibody followed by AF647 anti-mIgG H+L. Anti-Adeno-associated Virus 9, clone HL2372, supplied by Merck, Cat #MABF2309-100UL, 1:500 dilution. Detection antibody: AlexaFluor647 anti-mIgG H+L.

FIG. 9A-FIG. 9C FIG. 9A FIG. 9B display non-limiting exemplary data related to confirmation and specificity screens. Controls were added to fixed confirmation slides using the direct fix method (samples not removed). Anti-Adeno-associated Virus 9, clone HL2372, supplied by Merck, Cat #MABF2309-100UL, 1:500 dilution. Detection antibody: AlexaFluor647 anti-mIgG H+L.

FIG. 10 displays data related to overall summary of specific hits (e.g., weak intensity and above).

FIG. 11 displays data related to overall summary of specific hits (e.g., weak intensity and above).

FIG. 12A-FIG. 12E display non-limiting exemplary data showing high-throughput screen identifies AAV-binding human proteins. FIG. 12A displays a schematic of AAV cell microarray screen. DNA oligos that encode individual membrane proteins were chemically coupled to slides in a known pattern, the cells that grow on them were reverse transfected, thereby creating spots of cells overexpressing a particular, known protein. Each protein was expressed in duplicate at two different slide locations on the slide. When AAVs were applied to the slides, enhanced binding was detected from duplicate cell spots overexpressing cognate AAV receptors. FIG. 12B displays data related to known AAV capsid receptor interactions, such as AAVR and LY6A with AAV-PHP.eB, which were used to optimize conditions for streptavidin-based detection of biotinylated capsids with two sets of replicate spots. Anti-TGFBR2 antibody was used as a non-AAV positive control. FIG. 12C shows the use of AAVR and LY6A interaction with AAV9.CAP-B22 to optimize conditions for anti-AAV9 antibody direct detection of unmodified capsids with two sets of replicate spots. Anti-TGFBR2 antibody was used as a non-AAV control. FIG. 12D shows the pooled AAV capsid screening conditions were optimized by varying the concentrations of individual capsids within the pool to maximize signal to noise after direct detection with anti-AAV9 antibody, with two sets of replicate spots. FIG. 12E displays data showing pooled screening led to preliminary hits which were deconvoluted by individual-capsid screens. Novel potential capsid-binding proteins were identified by direct detection with anti-AAV9 antibody. Transfection control condition detected fluorescent protein reverse transfected along with each receptor. The none condition was treated only with anti-AAV9 antibody. DPP4, IL3, and DKK3 were identified in all individual AAV screens, and likely represent interactions outside the engineered regions of AAV9. LY6A, GP2, LRP6, FAM234A, ANPEP, CSF2, and EPYC, specifically bind to at least one engineered capsid.

FIG. 13A-FIG. 13D display non-limiting exemplary data related to species and serotype-specific interaction between AAV9 and IL3. FIG. 13A displays a schematic of Surface Plasmon Resonance (SPR) experiments where IL3-Fc is captured on a protein A sensor chip and AAV analyte flows over the sensor. FIG. 13B displays graphs of data showing SPR confirms serotype-specific interaction of AAV9 with the human immunomodulatory cytokine IL3. FIG. 13C displays graphs of data showing SPR confirms AAV9 binding with macaque but not marmoset or mouse IL3. FIG. 13D displays data related to MS/MS analyses. Bis(sulfosuccinimidyl)suberate (BS3) crosslinked AAV9 and hsIL3 were extracted from a PAGE gel and analyzed by MS/MS, identifying two intermolecular crosslinks with high-confidence XlinkX scores.

FIG. 14A-FIG. 14D display non-limiting exemplary data showing primate brain-enhanced AAVs gain interaction with LRP6. FIG. 14A shows non-limiting exemplary cartoons, showing that arraying AAV capsid-specific hits by human brain endothelial cell expression levels reveals highly-conserved LRP6 as a potential receptor for BBB crossing. FIG. 14B displays graphs showing SPR confirms that the engineered capsids AAV9-X1.1 and CAP-Mac gained direct binding interactions with human LRP6. FIG. 14C shows of AlphaFold models of X1 and CAP-Mac peptides which predict selective interaction with human LRP6 YWTD domain 1 (E1). FIG. 14D displays SPR data of mouse LRP6-E1E2 and LRP6-E3E4 (the minimal stable extracellular domain fragments due to cooperative folding) confirms that AAV9-X1.1 and CAP-Mac bind only to LRP6-E1E2.

FIG. 15A-FIG. 15F display non-limiting exemplary data showing LRP6 modulates CNS function of engineered AAVs in mice. FIG. 15A displays a schematic of Lrp6 conditional knockout by sequential AAV injection. Lrp6 Cre-conditional knockout mice are systemically injected with AAV1-X1 packaging either Cre or mCherry, creating cohorts of mice that differ in their LRP6 expression. After allowing time for expression, these cohorts were each injected with AAV9-PHP.eB or AAV9-X1.1 packaging eGFP. By switching serotypes, neutralizing antibodies are evaded and vector dependence on Lrp6 in vivo may be assessed. FIG. 15B displays representative sagittal brain images and liver images from the conditional Lrp6 knockout experiment. Imaging parameters were optimized independently for AAV9-X1.1 and AAV9-PHP.eB second dose conditions. FIG. 15C shows graphs of quantification of AAV potency demonstrating that conditional knockout of Lrp6 in mouse brain selectively and potently reduces AAV9-X1.1 brain and liver potency. Data points are the average of two sections per tissue region for an animal, with consistent physiological regions of interest across the four experimental cohorts. Bars represent the mean value. FIG. 15D-FIG. 15E display data showing that AAV9-X1.1 has enhanced potency in macaque (FIG. 15D) and human primary brain microvascular endothelial cell culture (FIG. 15E), which decreases to AAV9 levels with Mesd inhibition of LRP6. Bars indicate the mean value. Immunofluorescence of mouse and infant macaque brain tissue reveals consistently high LRP6 expression across brain endothelia as well as neurons and astrocytes (FIG. 15F).

FIG. 16 shows non-limiting exemplary data related to individual characterization of pool AAVs prior to full screen. Individual AAVs were tested at various doses to determine the optimal signal to noise ratio for each capsid while confirming detection of known interactions KIAA0319L (AAVR) and, for CAP-B22 only, LY6A. AAV binding detected at duplicate spots of the same protein is indicated by arrows.

FIG. 17A-FIG. 17C display data related to cell culture potency assay validation of high-throughput screen hits. FIG. 17A depicts representative images and quantification showing transient overexpression of mouse and human GP2 in HEK293T cells resulted in enhanced potency for CAP-Mac and AAV9-X1.1, with a stronger effect for the human protein. Scales show extent of infection (min, 0.01; max, 0.17) and total brightness per signal area (min, 0.11; max, 0.45). FIG. 17B depicts representative images and quantification showing transient overexpression of mouse and human FAM234A in HEK293T cells results in enhanced potency for PHP.eB and CAP-B22, with a stronger effect for the mouse protein. Extent of infection (min, 0.04; max, 0.07) and total brightness per signal area (min, 0.16; max, 0.29). FIG. 17C depicts representative images and quantification showing transient overexpression of human ANPEP and DPP4 did not result in potency enhancements for AAV9-X1.1 and AAV9, respectively. Scale bars indicate 200 μm.

FIG. 18A-FIG. 18B display non-limiting exemplary data related to SPR confirmation of selected screen hits. Immobilization of human DKK3-Fc (FIG. 18A) or human GP2-Fc (FIG. 18B) on a protein A chip allowed AAV analyte interactions to be assessed. In contrast to the cell microarray screen, no interaction was observed for AAV9 with DKK3, whereas AAV9-X1.1 gained direct binding ability to human GP2, in agreement with the cell microarray screen and cell culture potency assay.

FIG. 19A-FIG. 19B display non-limiting exemplary graphs of annotated MS/MS spectra of AAV9 crosslinked to human IL3 via BS3.

FIG. 20A-FIG. 20D display non-limiting exemplary data related to LRP6 binding to X1 peptide in multiple serotypes and to AAV-BI30. FIG. 20A displays data showing SPR of the complete human LRP6 extracellular domain confirmed that the X1 insertion peptide modularly enables LRP6 binding across multiple serotypes. FIG. 20B displays data showing SPR of AAV-BI30 confirmed binding interaction with mouse LRP6-E1E2 and not LRP6-E3E4. FIG. 20C shows exemplary AlphaFold models that predict that X1 and CAP-Mac variable region VIII peptides bind to WYTD domains 1 or 2 (e.g., E1E2) but, in some embodiments, cannot confidently assign a specific binding pose. FIG. 20D displays non-limiting exemplary data showing that despite their high degree of sequence similarity, LRP5 WYTD domain 1, unlike that of LRP6, does not bind AAV-X1.1.

FIG. 21 depicts non-limiting exemplary data showing X1.1 and CAP-Mac bind LRP6 and AAVR but not LRP5. Pull-down assay with the extracellular domains of mouse LRP6, mouse LRP5, and human AAVR PDK2 domain against AAV9, AAV9-X1.1, and CAP-Mac prey is shown. Asterisks indicate the LRP6 binding interaction gained by X1.1 and CAP-Mac during directed evolution from parent capsid AAV9.

FIG. 22A-FIG. 22B display non-limiting exemplary data related to cell culture potency assay validation of LRP6 interaction. FIG. 22A shows a schematic of Mesd chaperone function and LRP6 domain-dependent inhibition by recombinant Mesd and SOST proteins. FIG. 22B shows quantification of AAV potency demonstrating the effects of LRP receptor transient overexpression and LRP6 inhibition. Extent of infection (min, 0.04; max, 0.23) and total brightness per signal area (min, 0.04; max, 0.51).

FIG. 23A-FIG. 23B display non-limiting exemplary data related to the potency of X1 AAVs in mouse liver and human primary cell culture. FIG. 23A shows representative liver images from the conditional Lrp6 knockout experiment. The same imaging parameters were applied to all conditions. FIG. 23B depicts data showing AAV1-X1 has enhanced potency in human primary brain microvascular endothelial cell culture, which decreases to AAV1 levels with Mesd inhibition of LRP6. Bars indicate the mean value.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of increasing permeability of the blood brain barrier. In some embodiments, the method comprises: providing a targeting peptide capable of binding to low density lipoprotein receptor related protein 6 (LRP6), thereby increasing permeability of the blood brain barrier.

Disclosed herein include methods of delivering a payload to a nervous system of a subject. In some embodiments, the method comprises: providing a targeting peptide capable of binding to LRP6 or a derivative thereof, wherein the targeting peptide is part of a delivery system, and wherein the delivery system comprises the payload to be delivered to the nervous system; and administering the delivery system to the subject.

Disclosed herein include adeno-associated virus (AAV) capsid proteins. In some embodiments, the AAV capsid protein comprises a targeting peptide having a binding specificity to LRP6. Disclosed herein include recombinant adeno-associated virus (rAAV). In some embodiments, the rAAV comprises any of the AAV capsid protein disclosed herein. In some embodiments, the rAAV comprises an AAV capsid protein which comprises a targeting peptide having a binding specificity to LRP6, wherein the amino acid sequence of the targeting peptide is inserted between two adjacent amino acids in AA587-594, or functional equivalents thereof, of the AAV capsid protein.

Disclosed herein include compositions for use in the delivery of an agent to a nervous system of a subject in need. In some embodiments, the composition comprises an AAV comprising (1) an AAV capsid protein disclosed herein and (2) an agent to be delivered to the nervous system of the subject; optionally wherein the nervous system is the central nervous system (CNS), the peripheral nervous system (PNS), or a combination thereof.

Disclosed herein include antibodies or fragments thereof. In some embodiments, the antibody or fragment thereof comprises an amino acid sequence having a binding specificity to LRP6. Disclosed herein include peptides or a derivative or a conjugate thereof, having specificity to LRP6. Disclosed herein include nucleic acids. In some embodiments, the nucleic acid comprises a sequence encoding any of the antibody or fragment thereof or the peptide or any of the derivative or the conjugate thereof disclosed herein.

Disclosed herein include delivery systems. In some embodiments, the delivery system comprises (1) a targeting peptide having specificity to low density lipoprotein receptor related protein 6 (LRP6); and (2) an agent.

Disclosed herein include methods of designing a targeting peptide having specificity to LRP6. In some embodiments, the method comprises: generating in silico one or more targeting peptides each capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, 5114, E115, R141, W157, W183, or W242 in LRP6 having an amino acid sequence of SEQ ID NO: 31.

Disclosed herein include agents capable of binding to a protein selected from the group consisting of interleukin 3 (IL3), Family With Sequence Similarity 234 Member A (FAM234A), Glycoprotein 2 (GP2), Dipeptidyl peptidase-4 (DPP4), Dickkopf WNT Signaling Pathway Inhibitor 3 (DKK3), Alanyl Aminopeptidase (ANPEP), Epiphycan (EPYC), and LRP6.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see e.g., Lewin, “Genes V” (Oxford University Press, Oxford) pages 847-873). Exemplary regulatory elements in prokaryotes include promoters, operator sequences and a ribosome binding site. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, the term “variant” can refer to a polynucleotide or polypeptide having a sequence substantially similar to a reference polynucleotide or polypeptide. In the case of a polynucleotide, a variant can have deletions, substitutions, and/or additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

As used herein, the term “binding” refers to a non-covalent interaction between macromolecules (e.g., between two proteins and/or peptides). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

The wording “specific,” “specifically,” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred. The term “specific,” “specifically,” or “specificity” as used herein with reference to a binding of a targeting peptide to a target protein, e.g., LRP6, refers to the ability of the targeting peptide to form stable complex with a target protein, with substantially less to no binding to macromolecules other than the target protein that may be present. The term “specific,” “specifically,” or “specificity” as used herein with reference to a binding of a targeting peptide to a target protein, e.g., LRP6, also refers to the ability of the protein to form stable complex with the targeting peptide, with substantially less to no binding to candidate peptides other than the identified targeting peptide that may be present.

The term “AAV” or “adeno-associated virus” refers to a Dependoparvovirus within the Parvoviridae genus of viruses. For example, the AAV can be an AAV derived from a naturally occurring “wild-type” virus, an AAV derived from an rAAV genome packaged into a capsid derived from capsid proteins encoded by a naturally occurring cap gene and/or an rAAV genome packaged into a capsid derived from capsid proteins encoded by a non-natural capsid cap gene. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infects primates. Likewise, an AAV may infect bovine animals (e.g., “bovine AAV”, and the like). In some instances, the AAV is wild type, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV inverted terminal repeat (ITR) sequences. The AAV genome can be an rAAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV ITR sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

The terms “native” and “wild type” are used interchangeably herein, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject disclosure include not only the specifically exemplified full-length sequences, but also portions, segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA can be introduced exogenously by a virus, e.g., an AAV nanoparticle.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus, the term “antibody” includes any protein or peptide containing molecule that comprises at least a portion of an immunoglobulin molecule having biological activity of binding to the antigen. Examples of such include, but are not limited to, a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

As described herein, the products of natural evolution and of directed evolution on adeno-associated viruses (AAVs) were used to reverse engineer novel targets that determine therapeutic biodistribution. The identified targets open novel paths for rational engineering of potent and specific therapeutics in humans. A cell-based microarray screening technology to probe AAV interactions with human cell surface and secreted proteins was used. Screening revealed a panel of novel protein interactions grouped as (1) those targeting AAV serotype 9 (AAV9) and (2) those selectively targeting engineered AAVs evolved from AAV9. These new targets include proteins that can enhance therapeutic targeting to specific cell types and tissues, as well as targets that assist proteins in evading host immune responses. The disclosed target proteins newly allows target-based engineering to introduce and/or modulate interactions with these proteins to alter therapeutic biodistribution, efficacy, and patient immune response.

Provided herein are novel targets (e.g., of viral vectors) GP2, DPP4, IL-3, DKK3, FAM234A, LRP6, ANPEP, CSF2, and EPYC which can be used for modifying the targeting of biologics or chemicals in vitro or in vivo following intravenous, intrathecal, direct or other routes of administration. In some embodiments, GP2, DPP4, IL-3, DKK3, FAM234A, LRP6, ANPEP, CSF2, and/or EPYC can be used for crossing the BBB in mammalian systems by biological or chemical molecules.

Provided herein are AAVs whose capsids have been engineered directly against GP2, DPP4, IL-3, DKK3, FAM234A, LRP6, ANPEP, CSF2, and/or EPYC or which have been combined with molecules that have been engineered directly against GP2, DPP4, IL-3, DKK3, FAM234A, LRP6, ANPEP, CSF2, and/or EPYC.

Provided herein are antibodies, scFab, scFv and/or alternative protein scaffolds engineered directly against GP2, DPP4, IL-3, DKK3, FAM234A, LRP6, ANPEP, CSF2, and/or EPYC. Provided herein are peptides engineered directly against GP2, DPP4, IL-3, DKK3, FAM234A, LRP6, ANPEP, CSF2, and/or EPYC. Provided herein are small molecules engineered directly against GP2, DPP4, IL-3, DKK3, FAM234A, LRP6, ANPEP, CSF2, and/or EPYC.

Provided herein are small molecule- and biologic-drug conjugates (e.g., antibody-drug conjugate, ADC) targeted to GP2, DPP4, IL-3, DKK3, FAM234A, LRP6, ANPEP, CSF2, and/or EPYC. Provided herein are bispecific antibodies containing at least one Fab targeted to GP2, DPP4, IL-3, DKK3, FAM234A, LRP6, ANPEP, CSF2, and/or EPYC.

As disclosed herein, AAV capsids engineered directly against GP2, DPP4, IL-3, DKK3, FAM234A, LRP6, ANPEP, CSF2, and/or EPYC can be used for delivery of DNA or ASO molecules, therapeutic proteins, small therapeutic molecules, or other biologics in vitro and in vivo.

Disclosed herein include agents capable of binding to a protein selected from the group consisting of interleukin 3 (IL3), Family With Sequence Similarity 234 Member A (FAM234A), Glycoprotein 2 (GP2), Dipeptidyl peptidase-4 (DPP4), Dickkopf WNT Signaling Pathway Inhibitor 3 (DKK3), Alanyl Aminopeptidase (ANPEP), Epiphycan (EPYC), and LRP6. In some embodiments, the agent is selected from the group consisting of an antibody or fragment thereof, an aptamer, a small molecule, a nucleic acid, and a peptide.

The antibody or fragment thereof can comprise an Fc domain. The antibody or fragment thereof can be a single-chain variable fragment (scFv), a single-domain antibody, an immunoglobulin molecule, a monoclonal antibody, a chimeric antibody, a CDR-grafted antibody, a humanized antibody, a Fab fragment, a Fab′ fragment, a F(ab′)2 fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof. The nucleic acid can be a miRNA, an shRNA, and siRNA, or an oligonucleotide.

The agent can be conjugated to a detectable label. In some embodiments, the detectable label is selected from the group consisting of biotin, a fluorophore, a luminescent or bioluminescent marker, a radiolabel, an enzyme, an enzyme substrate, a quantum dot, an imaging agent, a metal particle, a magnetic particle, and any combination thereof. The agent can be a therapeutic agent.

The agent can be a targeting peptide. The targeting peptide can be part of a delivery system wherein the delivery system comprises a payload to be delivered to a cell. The delivery system can comprise a viral or a non-viral vector. The viral vector can comprise an AAV vector. The targeting peptide can be part of a capsid protein of an AAV vector. In some embodiments, the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof. The non-viral vector can comprise lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

The payload to be delivered the cell can be a biological molecule, a non-biological molecule, or a combination thereof. In some embodiments, the biological molecule is selected from the group consisting of a nucleic acid sequence, a protein, a peptide, a lipid, a polysaccharide, and any combination thereof. The payload can be a therapeutic molecule. The nucleic acid sequence to be delivered to a nervous system can comprise one or more of: a) a sequence encoding a trophic factor, a growth factor, or other soluble factors that might be released from the transduced cells and affect the survival or function of that cell and/or surrounding cells; b) a DNA (e.g., a genomic or cDNA sequence) that restores protein function to humans or animals harboring a genetic mutation(s) in that gene; c) a DNA that encodes a protein that can be used to control or alter the activity or state of a cell; d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

### Lipoprotein Receptor Related Protein 6 (LRP6)

Disclosed herein include methods of increasing permeability of the blood brain barrier. In some embodiments, the method comprises: providing a targeting peptide capable of binding to low density lipoprotein receptor related protein 6 (LRP6), thereby increasing permeability of the blood brain barrier. In some embodiments, the targeting peptide binds YWTD domain 1 and/or domain 2 (also referred to as E1 and E2 domains) of LRP6. The permeability of the blood brain barrier can be increased by at least, or at least about, 25%, 50%, 75%, 100%, or more (e.g., at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) as compared to the absence of the targeting peptide.

Disclosed herein include methods of delivering a payload to a nervous system of a subject. In some embodiments, the method comprises: providing a targeting peptide capable of binding to LRP6 or a derivative thereof, wherein the targeting peptide is part of a delivery system, and wherein the delivery system comprises the payload to be delivered to the nervous system; and administering the delivery system to the subject. The delivery system can comprise nanoparticles, nanotubes, nanowires, dendrimers, liposomes, ethosomes and aquasomes, polymersomes and niosomes, foams, hydrogels, cubosomes, quantum dots, exosomes, macrophages, and any combination thereof. The delivery system can comprise a viral vector or a non-viral vector.

In some embodiments, the targeting peptide enhances the binding affinity of the viral vector or the non-viral vector to LRP6. For example, the binding affinity of the viral vector to LRP6 can be enhanced by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). The viral vector can comprise an AAV vector. The targeting peptide can be part of a capsid protein of an AAV vector. In some embodiments, the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof. The non-viral vector can comprise lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

The payload to be delivered to a nervous system can be a biological molecule, a non-biological molecule, or a combination thereof. In some embodiments, the biological molecule is selected from the group consisting of a nucleic acid sequence, a protein, a peptide, a lipid, a polysaccharide, and any combination thereof. The payload can be a therapeutic molecule. The nucleic acid sequence to be delivered to a nervous system can comprise one or more of: a) a sequence encoding a trophic factor, a growth factor, or other soluble factors that might be released from the transduced cells and affect the survival or function of that cell and/or surrounding cells; b) a DNA (e.g., genomic DNA or cDNA sequence) that restores protein function to humans or animals harboring a genetic mutation(s) in that gene; c) a DNA that encodes a protein that can be used to control or alter the activity or state of a cell; d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

LRP6 gene encodes a member of the low density lipoprotein (LDL) receptor gene family. LDL receptors are transmembrane cell surface proteins involved in receptor-mediated endocytosis of lipoprotein and protein ligands. The protein encoded by this gene functions as a receptor or, with Frizzled, a co-receptor for Wnt and thereby transmits the canonical Wnt/beta-catenin signaling cascade. Through its interaction with the Wnt/beta-catenin signaling cascade this gene plays a role in the regulation of cell differentiation, proliferation, and migration and the development of many cancer types. This protein undergoes gamma-secretase dependent RIP-(regulated intramembrane proteolysis) processing. The NCBI Gene ID is 4040. The Ensembl ID is ENSG00000070018. The OMIM® ID is 603507 The UniProtKB/Swiss-Pro ID is 075581.

The LRP6 can be a mouse LRP6. In some embodiments, the LRP6 has an amino acid sequence having at least 80% (e.g., at least or at least about 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence of SEQ ID NO: 29. The LRP6 can be a macaque LRP6. In some embodiments, the LRP6 has an amino acid sequence having at least 80% (e.g., at least or at least about 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence of SEQ ID NO: 30. The LRP6 can be a human LRP6. In some embodiments, the LRP6 has an amino acid sequence having at least 80% (e.g., at least or at least about 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence of SEQ ID NO: 31.

The method disclosed herein can comprise: providing a targeting peptide capable of binding to LRP6 or a derivative thereof. Upon binding the targeting peptide can be capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31. The targeting peptide can be inserted between two adjacent amino acids in AA587-594 of SEQ ID NO: 11 of the AAV9 vector or functional equivalents of AA587-594 in an amino acid sequence at least 80% identical to SEQ ID NO: 11. The targeting peptide can be inserted between AA588-589 of SEQ ID NO: 11 of the AAV9 vector or functional equivalents of AA588-589 in an amino acid sequence at least 80% identical to SEQ ID NO: 11. The targeting peptide can be inserted between or replace AA452-460 of SEQ ID NO: 11 of the AAV9 vector or functional equivalents of AA588-589 in an amino acid sequence at least 80% identical to SEQ ID NO: 11.

In some embodiments, the targeting peptide can comprise or consist of an amino acid sequence having at least 80% identity (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 1-10. In some embodiments, the targeting peptide comprises or consists of an amino acid sequence of any of the sequences of SEQ ID NOs: 1-10. In some embodiments, the targeting peptide comprises at least four contiguous amino acids from any one of the sequences of SEQ ID NOs: 1-10.

### Targeting Peptides

Disclosed herein are targeting peptides and associated AAV particles comprising a capsid protein with one or more targeting peptide inserts, for enhanced or improved transduction of a target tissue (e.g., cells of the CNS or PNS). In some embodiments, the targeting peptide may direct an AAV particle to a cell or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei. In some embodiments, the targeting peptide may direct an AAV particle to a cell or tissue of the PNS. The cell or tissue of the PNS may be, but is not limited to, a dorsal root ganglion (DRG). The targeting peptide may direct an AAV particle to the CNS (e.g., the cortex) after intravenous administration. The targeting peptide may direct and AAV particle to the PNS (e.g., DRG) after intravenous administration.

A targeting peptide may vary in length. In some embodiments, the targeting peptide is 3-20 amino acids in length. As non-limiting examples, the targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length.

In some embodiments, there is provided a targeting peptide capable of binding to low density lipoprotein receptor related protein 6 (LRP6) or a derivative thereof. Upon binding the targeting peptide can be capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

Targeting peptides of the present disclosure may be identified and/or designed by any method known in the art. As a non-limiting example, the CREATE system as described in Deverman et al., (Nature Biotechnology 34(2):204-209 (2016)) and in International Patent Application Publication Nos. WO2015038958, WO2017100671, and WO2020028751 the contents of each of which are herein incorporated by reference in their entirety, may be used as a means of identifying targeting peptides, in either mice or other research animals, such as, but not limited to, non-human primates. The targeting peptides may be designed in silico. The in silico designed peptides can be tested in any in vitro or in vivo model as assessed by the skilled artisan.

Targeting peptides and associated AAV particles may be identified from libraries of AAV capsids comprised of targeting peptide variants. In some embodiments, the targeting peptides may be 7 amino acid sequences (7-mers). In another embodiment, the targeting peptides may be 9 amino acid sequences (9-mers). The targeting peptides may also differ in their method of creation or design, with non-limiting examples including, random peptide selection, site saturation mutagenesis, and/or optimization of a particular region of the peptide (e.g., flanking regions or central core).

In some embodiments, a targeting peptide library comprises targeting peptides of 7 amino acids (7-mer) in length randomly generated by PCR. In some embodiments, a targeting peptide library comprises targeting peptides with 3 mutated amino acids. In some embodiments, these 3 mutated amino acids are consecutive amino acids. In some embodiments, these 3 mutated amino acids are not consecutive amino acids. In some embodiments, the parent targeting peptide is a 7-mer. In another embodiment, the parent peptide is a 9-mer.

In some embodiments, a targeting peptide library comprises 7-mer targeting peptides, wherein the amino acids of the targeting peptide and/or the flanking sequences are evolved through site saturation mutagenesis of 3 consecutive amino acids. In some embodiments, NNK (N=any base; K=G or T) codons are used to generate the site saturated mutation sequences.

In Silico Screening of Targeting Peptides

Disclosed herein include methods of designing a targeting peptide having specificity to LRP6. In some embodiments, the method comprises: generating in silico one or more targeting peptides each capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, 5114, E115, R141, W157, W183, or W242 in LRP6 having an amino acid sequence of SEQ ID NO: 31.

Generating in silico the one or more targeting peptides can comprise: generating in silico a plurality of candidate peptides; performing computer-assisted docking simulations for each of the plurality of candidate peptides binding to LRP6; and analyzing the structure of LRP6 binding to one or more of the plurality of candidate peptides to identify one or more targeting peptides capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

In silico methods can offer a high throughput approach for screening a large number of candidate peptides and identifying targeting peptides with the desired specificity. In some embodiments, the candidate peptide can comprise a portion of an AAV capsid protein. In some embodiments, the candidate peptide is part of an AAV capsid protein. The method can comprise constructing one or more peptide-receptor models for each candidate peptide in complex with LRP6 (e.g., performing computer-assisted docking simulations). The molecular models can be constructed for a peptide-protein complex using any rational computational peptide design and docking methods, database, programs, or algorithms described herein or known in the field. Exemplary computational moldering methods, public database, and programs, include, but are not limited to, AlphaFold (e.g., AlphaFold2, and AlphaFold-Multimer provided by DeepMind, available at github.com/deepmind/alphafold), RoseTTAFold (github.com/RosettaCommons/RoseTTAFold), AutoDock, DOCK, FlexX, GOLD, OSPREY, SCWRL, PyMol, SWISS-MODEL (academic.oup.com/nar/article/46/W1/W296/5000024), Protein Data Bank (PDB) (available via web sites of member organizations, e.g., PDBe—pdbe.org, PDBj—pdbj.org, RC SB—resb.org/pdb, and BMRB—bmrb.wisc.edu), Phyre2 (nature.com/articles/nprot.2015.053), and RaptorX (nature.com/articles/nprot.2012.085).

The method can further comprise evaluating features or parameters associated with interactions between a candidate peptide and LRP6 using visualization software such as PyMol, Qlucore Omics Explorer, WebMol, Insight II, Discovery Studio 2.1 and others identifiable to a person skilled in the art. Features or parameters being evaluated can comprise interface energy and physical and geometric scorings. Evaluating the features or parameters associated with interactions between a candidate peptide and the LRP6 can comprise measuring surface complementarity, solvent accessible surface areas, solvation free energy, electrostatic interaction energy, van der Waals energy and/or the total molecular mechanics energy. The method can also comprise determining the total number of atoms in the interface, the total number of atoms in the peptide that are clashing with the LRP6, the binding angle of the peptide, and/or the binding depths of the peptide in each putative peptide-receptor complex model. The method can also comprise identifying the lowest energy conformation of a LRP6 complex. The energy score of each conformation can be determined by calculating the interaction energy between the peptide and LRP6, including electrostatic, desolvation, van der Waals energy as will be understood by a skilled person.

In some embodiments, the targeting peptides can be assigned with a binding score and ranked based on the binding score. A threshold can be imposed to identify desired targeting peptides. The method can comprise obtaining a binding score for each of the plurality of candidate peptides binding to LRP6 and selecting one or more of the plurality of candidate peptides having a binding score above a threshold value as a targeting peptide having a binding specificity (or high binding affinity) to LRP6.

A combination of physical and geometric scoring parameters including interface energy, binding angle, and binding pocket depth calculation can be used to generate a binding score. In some embodiments, the binding score for each of the plurality of candidate peptide sequences can be obtained by (1) counting a total number of atoms in the interface of a candidate peptide and LRP6; (2) counting a total number of atoms in the candidate peptide, wherein the atoms are clashing with LRP6; (3) obtaining a binding angle of the candidate peptide; and/or (4) obtaining a binding depth of the candidate peptide.

The total number of atoms in the interface of a candidate peptide and the LRP6 can be the total number of atoms within a cutoff distance between interfacing atoms of the candidate peptide and interfacing atoms of the LRP6. The cutoff distance can vary in different embodiments. In some embodiments, the cutoff distance is about, at most, at most about 5 angstrom (e.g., 2 angstrom, 3 angstrom, 4 angstrom or 5 angstrom). The number of interfacing atoms between a candidate peptide and LRP6 can be different in different embodiments. For example, the number of interfacing atoms can be about or can be at least 2, 3, 4, 5, 6, 7, 8, 9, 10, 11, 12, 13, 14, 15, 16, 17, 18, 19, 20, 25, 30, 35, 40, 45, 50, or a number or a range between any of these values.

The total number of atoms in the peptide that are clashing with LRP6 can be the total number of atoms within a clashing distance. A clashing distance can be defined as a distance when geometric clashes occur between peptide and receptor atoms. In some embodiments, a clashing distance can be about 1 angstrom.

The binding angle of the peptide can be defined as the angle between the vector from LRP6 gravity center to LRP6 anchor and the vector from LRP6 gravity center to peptide gravity center. The binding depth can be defined as the difference of the distance between the closest point on the peptide to LRP6 center and the minor radius of the ellipsoid hull of the LRP6 normalized by the minor radius. The binding score can be the sum of a contact score calculated based on the total number of atoms in the interface of a candidate peptide and the LRP6 and the total number of clashing atoms), a binding angle, and a binding depth. In some embodiments, a binding score is equal to or greater than 0. For example, a binding score is defined as 0, if the sum of a contact score, a binding angle and a binding depth is negative. The binding score of a targeting peptide binding to LRP6 can be about 0, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300 or a number or a range between any two of these values.

In some embodiments, the binding score of a candidate peptide (B^{peptide}) can be defined as follows:

$$\begin{matrix}{B}^{peptide} = max ( {B}_{energetic}^{peptide} + {B}_{angle}^{peptide} + {B}_{depth}^{prptide} , 0 ) & ( 1 ) \\ {B}_{energetic}^{peptide} = max ( {N}_{contact}^{peptide} - {10}^{3} \cdot {N}_{clash}^{peptide} , 0 ) & ( 2 ) \\ {B}_{angle}^{peptide} = \{ \begin{matrix}- {10}^{3} \cdot {( 1 - \frac{θ}{\frac{π}{2}} )}^{10} , if θ < \frac{π}{2} \\ 0 , if \frac{π}{2} <= θ <= π\end{matrix} & ( 3 ) \\ {B}_{depth}^{peptide} = {10}^{2} \cdot {10}^{3} & ( 4 )\end{matrix}$$

The method can further comprise selecting one or more of the plurality of candidate peptides having a binding score above a threshold value as a targeting peptide having a binding specificity to LRP6. The threshold can be an arbitrary value defined by a user. In some embodiments, the threshold can be a binding score of a targeting peptide known for having a binding specificity to LRP6. In some embodiments, results from individual pairwise competitions (a relative score between two competing peptides) can be assembled into a peptide competition metric that ranks sets of candidate peptides according to their receptor-binding probability encoded in the AlphaFold2 neural network.

CREATE System

In some embodiments, the targeting peptides of the present disclosure are isolated via the CREATE system, as described in Deverman et al., (Nature Biotechnology 34(2):204-209 (2016)) and in International Patent Application Publication Nos. WO2015038958 and WO2017100671, the contents of each of which are herein incorporated by reference in their entirety. “CREATE” or “Cre-recombinant-based AAV targeted evolution” refers to an AAV capsid selection strategy that selects for capsids that transduce target tissues (e.g., CNS or PNS) following intravenous injection. The method has been demonstrated in a mouse model.

Libraries of AAV capsids with one or more targeting peptide inserts are developed and administered intravenously to transgenic mice. These transgenic Cre-expressing mice may be developed for specific targeting, for example, GFAP-Cre mice may be used for targeting to astrocytes. In some embodiments, Cre/LoxP mediated system can be used to knock-out or over and/or ectopically express LRP6 to identify targeting peptides that interact with LRP6.

Variation of the targeting sequence as well as the transgenic animal model enables the selection of AAV variants with desired transduction profiles, for example, tropism to neurons or astrocytes, as compared to other AAV serotypes, including the parent AAV particle and capsid.

The CREATE method involves the generation of a library of targeting peptides which are then assembled into a viral genome backbone comprising a parent AAV capsid sequence. An AAV capsid library (AAV particles) is then generated, purified and administered to a transgenic animal (e.g., mouse). Target tissue is collected and AAV sequences selectively recovered from Cre expressing cells. These sequences are assessed and characterized for the identification of targeting peptides that lead to enrichment in a target tissue (i.e., enhanced transduction or tropism). Targeting peptides and associated AAV particles can then be generated for further testing and characterization. This process is considered one round of evolution or selection. In some embodiments, more than one round of evolution is conducted. As many as 15 rounds of selection may be conducted.

In more detail, the CREATE system uses an rAAV-Cap-in-cis-lox viral genome comprising AAV cap and regulator elements of the AAV rep genes and a Cre-invertible switch. Since this viral genome lacks a fully functioning rep gene necessary for AAV particle production, the rep is provided in trans. A modified AAV2/9 Rep-Cap plasmid may be provided, wherein stop-codons are provided in-frame to prevent the expression of VP1-VP3 proteins.

Capsid libraries are generated using the rAAV-Cap-in-cis-lox viral genome as a backbone. Targeting peptides are inserted into the parent AAV capsid protein (e.g., AAV9) at any position that results in the generation of a fully functional AAV capsid protein and AAV particle. Targeting peptides may be designed by any method known in the art. In some embodiments, targeting peptides are generated using polymerase chain reaction (PCR). AAV particles comprising capsid proteins with targeting peptide inserts are generated and viral genomes encoding a reporter (e.g., GFP) encapsulated within. These AAV particles (or AAV capsid library) are then administered to a transgenic mouse by intravenous delivery to the tail vein. Administration of these capsid libraries to Cre-expressing mice results in expression of the reporter payload in the target tissue, due to the expression of Cre.

AAV particles and/or viral genomes may be recovered from the target tissue for identification of targeting peptides and associated AAV particles that are enriched, indicating enhanced transduction of target tissue. Standard methods in the art, such as, but not limited to next generation sequencing (NGS), viral genome quantification, biochemical assays, immunohistochemistry and/or imaging of target tissue samples may be used to determine enrichment.

A target tissue may be any cell, tissue or organ of a subject. As non-limiting examples, samples may be collected from brain, spinal cord, dorsal root ganglia and associated roots, liver, heart, gastrocnemius muscle, soleus muscle, pancreas, kidney, spleen, lung, adrenal glands, stomach, sciatic nerve, saphenous nerve, thyroid gland, eyes (with or without optic nerve), pituitary gland, skeletal muscle (rectus femoris), colon, duodenum, ileum, jejunum, skin of the leg, superior cervical ganglia, urinary bladder, ovaries, uterus, prostate gland, testes, and/or any sites identified as having a lesion, or being of interest.

Targeting peptides and associated AAV capsid proteins and AAV particles identified using a CREATE system include AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), and AAVPHP.S. In some embodiments, CREATE in mice is used to identify AAV capsids and/or targeting peptides having enhanced transduction of a target tissue (e.g., CNS or PNS).

The CREATE system has proven efficacious in identifying targeting peptides for enhanced transduction to the CNS of mice after intravenous administration. However, translation of findings from mouse to human is not always straightforward. Modifying the CREATE system for non-transgenic animals or model systems that more closely resemble humans may help identify targeting peptides and associated AAV capsids and particles useful for the treatment of human disease. In some embodiments, the AAV interactors identified herein can be used to address this unmet need and assess or generate new models for design of AAV capsids in humans.

For adaptation of the CREATE method to non-transgenic animals, another mechanism needs to be used to alter target tissues and/or cells to express Cre. In some embodiments, AAV Cre-vectors may be used to transduce cells and induce subsequent Cre expression. In some embodiments, these AAV Cre-vectors may be AAV1-Cre vectors. The AAV Cre-vectors can comprise viral genomes with a cell-type specific promoter. These cell-type specific promotors may be, but are not limited to, CAG, UBC, EF1α, synapsin, GFAP, MBP, VGLUT, VGAT, Nav1.8, parvalbumin, TH, ChaT, and/or any promoter known in the art.

In some embodiments, these AAV-Cre vectors are delivered to a target tissue by intraparenchymal administration. In some embodiments, the intraparenchymal administration is directly to the putamen of the subject. In some embodiments, the intraparenchymal administration is directly to the thalamus of a subject. In some embodiments, the intraparenchymal administration is directly to the cortex of a subject. In some embodiments, the intraparenchymal administration is indirectly to the cortex of a subject. In some embodiments, the intraparenchymal administration is simultaneously to one or more of the putamen, the thalamus and or the cortex of a subject, and may be bi-lateral administrations. In some embodiments, the subject is a non-human primate.

As for the CREATE method developed in mice, the AAV capsid libraries may be administered intravenously. In another embodiment, the AAV capsid libraries may be administered by intraparenchymal delivery. In some embodiments, the AAV capsid library is administered prior to the delivery of the AAV-Cre vectors. In another embodiment, the AAV capsid library is administered after the delivery of the AAV-Cre vectors. The length of time between the administration of the AAV-Cre vectors and the AAV capsid libraries may be seconds, minutes, hours, days, weeks, or years.

The AAV capsid library may comprise AAV particles comprising a viral genome encoding a reporter (e.g., GFP). Only those cells of the target tissue (e.g., CNS or DRG) also expressing Cre (co-transduced by a Cre-vector administered intraparenchymally) will express the reporter. Target tissues may be collected and analyzed for the identification of AAV particles and targeting peptides that lead to enrichment in the target tissue, i.e., enhanced transduction. Standard methods in the art may be used to assess, analyze, or characterize sample tissues and AAV sequences, including but not limited to, next generation sequencing, viral genome quantification, biochemical assays, immunohistochemistry and/or imaging.

### Antibody and Peptide Derivatives

Disclosed herein also include antibodies or fragments thereof comprising an amino acid sequence having a binding specificity to a target protein disclosed herein (e.g., LRP6). Disclosed herein also includes a peptide derivative or a conjugate thereof, having specificity to a protein disclosed herein (e.g., LRP6).

In some embodiments, the antibody or fragment thereof can further comprise an Fc domain. In some embodiments, the antibody or fragment thereof is a single-chain variable fragment (scFv), a single-domain antibody, an immunoglobulin molecule, a monoclonal antibody, a chimeric antibody, a CDR-grafted antibody, a humanized antibody, a Fab fragment, a Fab′ fragment, an F(ab′)2 fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof.

In some embodiments, the antibody or fragment thereof is a bispecific antibody comprising at least one Fab having specificity to LRP6 or another protein identified herein (e.g., GP2). The bispecific antibody can comprise another binding site directed at a different antigen.

In some embodiments, the antibodies or fragments thereof are not capable of eliciting a deleterious immune response in a subject to be treated, e.g., in a human. In some embodiments, antibodies, fragments, variants, or derivatives thereof of the disclosure are modified to reduce their immunogenicity using techniques recognized in the art. For example, antibodies can be humanized, primatized, deimmunized, or chimeric antibodies.

In some embodiments, the antibodies, fragments, variants, or derivatives thereof can further comprise a chemical moiety not naturally associated with an antibody. For example, the antibody or fragment thereof can comprise a flexible linker or can be modified to add a functional moiety such as a detectable label. The antibodies, fragments, variants, or derivatives thereof can be modified, e.g., by the covalent or non-covalent attachment of a chemical moiety to the antibody such that the attachment does not interfere or prevent the antibody from binding to the epitope. In some embodiments, a chemical moiety can be conjugated to an antibody using any technique known in the art.

The present disclosure also provides isolated polynucleotides or nucleic acid molecules encoding the peptides, antibodies, fragments, variants or derivatives thereof of the disclosure. For example, the polynucleotides of the present disclosure can encode the heavy and light chain variable regions of the antibodies, fragments, variants or derivatives thereof on the same polynucleotide molecule or on separate polynucleotide molecules. In some embodiments, the polynucleotides of the present disclosure can encode portions of the heavy and light chain variable regions of the antibodies (e.g., the CDR regions), fragments, variants or derivatives thereof on the same polynucleotide molecule or on separate polynucleotide molecules.

### Payload Delivery

Disclosed herein include methods and delivery systems for delivering a payload (e.g., a therapeutic agent) to a target tissue, e.g., a nervous system. In some embodiments, the method comprises providing a targeting peptide capable of binding LRP6 or a derivative thereof. The targeting peptide can be part of a delivery system and the delivery system can comprise a payload to be delivered to a nervous system. The method can further comprise administering the delivery system to the subject.

In some embodiments, the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a nanoparticle selected from lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

In some embodiments, the delivery system comprises a viral vector or a non-viral vector. For example, the viral vector can comprise an adenovirus vector, an adeno-associated virus (AAV) vector, a lentiviral vector, or a retrovirus vector. In some embodiments, the viral vector is an AAV vector and the target peptide can be part of a capsid protein of the AAV vector.

Adeno-Associated Virus (AAV) and Recombinant AAV (rAAV)

In some embodiments, the delivery system for delivering a payload across the BBB is an AAV vector. In some embodiments, the AAV is a replication-deficient parvovirus, the single-stranded DNA genome of which is about 4.7 kb in length including 145 nucleotide inverted terminal repeats (ITRs). The ITRs play a role in the integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for the production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

In some embodiments, the AAV vectors can comprise coding regions of one or more proteins of interest. The AAV vector can include a 5′ AAV ITR, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the AAV vector includes a posttranscriptional regulator-element downstream of the restriction site and upstream of the 3′ AAV ITR.

The viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in US2012/0232133 which is hereby incorporated by reference in its entirety.

The AAV serotype used to derive the AAV capsid protein can vary. The AAV capsid can be derived from AAV9, or a variant thereof. The AAV capsid can be derived from an AAV selected from AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. In some embodiments, the AAV capsid protein can be derived from an AAV serotype selected from AAV9, AAV9 K449R (or K449R AAV9), AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 A1 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh. 54, AAV4-19/rh.55, AAV5-3/rh.57, AAV5-22/rh. 58, AAV7.3/hu.7, AAV16.8/hu.10, AAV16.12/hu.1 1, AAV29.3/bb.1, AAV29.5/bb.2, AAV106. 1/hu.37, AAV1 14.3/hu.40, AAV127.2/hu.41, AAV127.5/hu.42, AAV128.3/hu.44, AAV130.4/hu.48, AAV145. 1/hu.53, AAV145.5/hu.54, AAV145.6/hu.55, AAV161. 10/hu.60, AAV161.6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh. 57, AAVrh. 58, AAVrh. 61, AAVrh. 64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1 0.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

The AAV vector can be an AAV9 having an amino acid sequence of SEQ ID NO: 11 or an amino acid sequence having at least 70% sequence identity (e.g., at least 70%, 80%, 90%, 95%, 96%, 97%, 98%, 99% or higher) to an amino acid sequence of SEQ ID NO: 11. In some embodiments, the AAV vector is a variant AAV vector having at least 70% sequence identity (e.g., at least 70%, 80%, 90%, 95%, 96%, 97%, 98%, 99% or higher) to an amino acid sequence of any one of SEQ ID NOs: 11-18.

In some embodiments, the AAV vectors disclosed herein can be used as AAV transfer vectors carrying a transgene encoding a protein of interest (e.g., a targeting peptide) for producing recombinant AAV viruses that can express the protein of interest in a host cell. Accordingly, disclosed herein also include recombinant AAV viruses (rAAV). The rAAV can comprise an AAV capsid protein described herein.

The rAAV can comprise a chimeric AAV capsid. A “chimeric” AAV capsid refers to a capsid that has an exogenous amino acid or amino acid sequence. The rAAV may comprise a mosaic AAV capsid. A “mosaic” AAV capsid refers to a capsid that made up of two or more capsid proteins or polypeptides, each derived from a different AAV serotype. The rAAV can be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype ban be pseudotyped, which means that the ITRs from a first AAV serotype (e.g., AAV1) are used in a capsid from a second AAV serotype (e.g., AAV9), wherein the first and second AAV serotypes are not the same. As a non-limiting example, a pseudotyped AAV serotype comprising the AAV1 ITRs and AAV9 capsid protein may be indicated AAV1/9. The rAAV may additionally, or alternatively, comprise a capsid that has been engineered to express an exogenous ligand binding moiety (e.g., receptor), or a native receptor that is modified.

In some embodiments, the rAAV capsid proteins comprise a substitution or insertion of one or more amino acids in an amino acid sequence of an AAV capsid protein. The rAAV capsid proteins described herein have, in some cases, an insertion or substitution of an amino acid that is heterologous to the wild-type AAV capsid protein at the amino acid position of the insertion or substitution. In some embodiments, the amino acid is not endogenous to the wild-type AAV capsid protein at the amino acid position of the insertion or substitution. The amino acid can be a naturally occurring amino acid in the same or equivalent amino acid position as the insertion of the substitution in a different AAV capsid protein. The AAV capsid protein from which the engineered AAV capsid protein of the present disclosure is produced can be referred to as a “parental” or “wild-type” AAV capsid protein, or a “corresponding unmodified capsid protein.” In some cases, the parental AAV capsid protein has a serotype selected from AAV1, AAV2, rAAV3, AAV4, AAV5, AAV6, AAV7, AAV8, AAV9, AAV10, AAV11, and AAV12. The complete genome of AAV-1 is provided in GenBank Accession No. NC 002077; the complete genome of AAV-2 is provided in GenBank Accession No. NC 001401 and Srivastava et al., J. Virol., 45: 555-564 (1983); the complete genome of AAV-3 is provided in GenBank Accession No. NC 1829; the complete genome of AAV-4 is provided in GenBank Accession No. NC 001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

In some embodiments, the rAAV vectors disclosed herein can carry a transgene encoding a targeting peptide described herein that is capable of binding LRP6. The targeting peptide can be part of a capsid of the rAAV. Disclosed herein also include AAV capsid proteins. The AAV capsid protein can comprise a targeting peptide disclosed herein.

The location of the targeting peptide within the capsid protein can vary. In some embodiments, the targeting peptide can be inserted between two adjacent amino acids in AA586-595 (e.g., between AA586 and AA587, AA587 and AA588, AA588 and AA589, AA589 and AA590, AA590 and AA591, AA591 and AA592, AA592 and AA593, AA593, AA594 and AA595) of AAV9 capsid protein or functional equivalents thereof in other AAV capsid proteins. The AAV vector can be a vector selected from AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, or a variant thereof. In some embodiments, the AAV vector is AAV9 or a variant or a derivative thereof. For example, the AAV capsid protein comprises, or consists thereof, SEQ ID NO: 11 or an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 11.

The targeting peptide can be inserted between AA588-589 of AAV9 capsid protein or functional equivalents thereof in other AAV capsid proteins. The two adjacent amino acids can be AA588-589. In some embodiments, the targeting peptide is inserted between AA587-590 of AAV9 capsid protein or functional equivalents thereof in other AAV capsid proteins.

Upon binding to LRP6, the targeting peptide can be capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

The binding specificity between the targeting peptide carried by the rAAV and the LRP6 can be screened using a multiplexed Cre-recombination-based AAV targeted evolution (CREATE) method (M-CREATE). In the M-CREATE, the rAAV nucleic acid contains a label sequence flanked by two lox sequences. The rAAV is administrated to an animal (e.g., mouse) with the gene encoding a Cre recombinase expressed only in a target cell (e.g., endothelial cells in brain). The label sequence of rAAV capable of entering the target cell is inverted by Cre recombinase expressed in the target cell. Because the nucleic acid of rAAV lacking specificity to the target cell has no access to Cre recombinase, its label sequence is not inverted. Thus, after sequencing the nucleic acid of rAAV recovered from the animal, the targeting peptide of rAAV containing an inverted label sequence has specificity to the target cell. M-CREATE method is described in detail in U.S. Patent Publication US20170166926A1, the content of which is incorporated herein by reference in its entirety for all purposes.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

Uses of AAV Vectors and rAAVs for Payload Delivery

Disclosed herein also includes compositions for use in the delivery of a payload (e.g., a pharmaceutical agent) to a target environment such as a nervous system of a subject. The composition can comprise an AAV comprising (1) an AAV capsid protein disclosed herein and (2) an agent to be delivered to a target environment (e.g., nervous system) of the subject.

The target environment can be the CNS, the peripheral nervous system (PNS), or a combination thereof. The target environment can be brain endothelial cells, neurons, capillaries in the brain, arterioles in the brain, arteries in the brain, or a combination thereof.

The pharmaceutical agent to be delivered can comprise a nucleic acid, a peptide, a small molecule, an aptamer, or a combination thereof. The AAV vectors disclosed herein can be effectively transduced to a target environment (e.g., the CNS), for example, for delivering nucleic acids. In some embodiments, a method of delivering a nucleic acid sequence to the nervous system is provided. The protein can be part of a capsid of an AAV. The AAV can comprise a nucleic acid sequence to be delivered to a nervous system. One can then administer the AAV to the subject.

The nucleic acid sequence to be delivered to a nervous system can comprise one or more of: a) a sequence encoding a trophic factor, a growth factor, or other soluble factors that might be released from the transduced cells and affect the survival or function of that cell and/or surrounding cells; b) a DNA (e.g., genomic or cDNA sequence) that restores protein function to humans or animals harboring a genetic mutation(s) in that gene; c) a DNA that encodes a protein that can be used to control or alter the activity or state of a cell; d) a DNA that encodes a protein or a nucleic acid used for assessing the state of a cell; e) a DNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

In some embodiments, the vector can also comprise regulatory control elements known to one of skill in the art to influence the expression of the RNA and/or protein products encoded by the polynucleotide within desired cells of the subject.

Functionally, expression of the polynucleotide can be at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of the intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in a specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences, the CMV, chicken β-actin, rabbit β-globin (CAG) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

The nucleic acid (e.g., a heterologous nucleic acid) can comprise a 5′ ITR and a 3′ ITR. The agent can comprise a DNA sequence encoding a protein (e.g., a trophic factor, a growth factor, or a soluble protein). The nucleic acid can comprise a promoter operably linked to the polynucleotide encoding, e.g., a protein or an RNA agent. The promoter can be capable of inducing the transcription of the polynucleotide. Transcription of the polynucleotide can generate a transcript. The nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the transcript and/or reducing the translation of the transcript. In some embodiments, the silencing effector comprises one or more miRNA binding sites (e.g., miR-122 binding sites). miRNA binding sites are operably linked regulatory elements that are typically located in the 3′UTR of the transcribed sequence. Binding of miRNAs to the target transcript (in complex with the RNA-Induced Silencing Complex, RISC) can reduce the expression of the target transcript via translation inhibition and/or transcript degradation.

The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The nucleic acid can be or can encode an RNA agent. The RNA agent can comprise one or more of dsRNA, siRNA, shRNA, pre-miRNA, pri-miRNA, miRNA, stRNA, lncRNA, piRNA, and snoRNA. The RNA agent inhibits or suppresses the expression of a gene of interest in a cell. In some embodiments, the gene of interest can be selected from SOD1, MAPT, APOE, HTT, C90RF72, TDP-43, APP, BACE, SNCA, ATXN1, ATXN2, ATXN3, ATXN7, SCN1A-SCN5A, and SCN8A-SCN11A. The nucleic acid further can comprise a polynucleotide encoding one or more secondary proteins, and the protein and the one or more secondary proteins can comprise a synthetic protein circuit. The nucleic acid can comprise a single-stranded AAV (ssAAV) vector or a self-complementary AAV (scAAV) vector.

The promoter can comprise a ubiquitous promoter. The ubiquitous promoter can be selected from a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin ((3-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, including but not limited to, a tetracycline responsive promoter, a TRE promoter, a Tre3G promoter, an ecdysone responsive promoter, a cumate responsive promoter, a glucocorticoid responsive promoter, and estrogen responsive promoter, a PPAR-y promoter, an RU-486 responsive promoter, or a combination thereof.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. The tissue specific promoter can be a neuron-specific promoter, for example a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be, or comprise, a muscle-specific promoter, e.g., an MCK promoter.

The promoter can comprise an intronic sequence. The promoter can comprise a bidirectional promoter and/or an enhancer. In some embodiments, the enhancer can be a CMV enhancer. One or more cells of a subject can comprise an endogenous version of a nucleic acid sequence (e.g., a gene), and the promoter can comprise or can be derived from the promoter of the endogenous version. In some embodiments, one or more cells of a subject comprise an endogenous version of the nucleic acid sequence, and the sequence is not truncated relative to the endogenous version.

The promoter can vary in length, for example be less than 1 kb. In other embodiments, the promoter is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of the therapeutic gene expression product for a period of time in targeted tissues such as, but not limited to, the CNS. Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 1 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

As used herein, a “protein of interest” can be any protein, including naturally-occurring and non-naturally occurring proteins. In some embodiments, a polynucleotide encoding one or more proteins of interest can be present in one of the AAV vectors disclosed herein, wherein the polynucleotide is operably linked with a promoter. In some instances, the promoter can drive the expression of the protein(s) of interest in a host cell (e.g., an endothelial cell). In some embodiments, the protein of interest is an anti-tau antibody, an anti-AB antibody, and/or ApoE isoform.

The protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof.

The protein can comprise a disease-associated protein. In some embodiments, the level of expression of the disease-associated protein correlates with the occurrence and/or progression of the disease. The protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

In some embodiments, the nucleic acid can comprise a DNA (e.g., a cDNA or genomic DNA sequence) that encodes a protein to control or monitor the activity or state of a cell, and/or for assessing the state of a cell. The protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The protein can comprise a nuclear localization signal (NLS) or a nuclear export signal (NES).

The protein can comprise a CRE recombinase, GCaMP, a cell therapy component, a knock-down gene therapy component, a cell-surface exposed epitope, or any combination thereof. The protein can comprise a chimeric antigen receptor. The protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

In some embodiments, the nucleic acid can comprise a DNA (e.g., a cDNA or genomic DNA sequence) that encodes a protein for gene editing, or a guide RNA; or a DNA sequence for genome editing via homologous recombination. The protein can comprise a programmable nuclease. In some embodiments, the programmable nuclease is selected from: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tetl, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The nucleic acid and/or rAAV can comprise a polynucleotide encoding (i) a targeting molecule and/or (ii) a donor nucleic acid. The targeting molecule can be capable of associating with the programmable nuclease. The targeting molecule can comprise single-strand DNA or single-strand RNA. The targeting molecule can comprise a single guide RNA (sgRNA).

The rAAV disclosed herein can comprise one or more of the nucleic acids disclosed herein. The nucleic acid can comprise a polynucleotide encoding a protein. The nucleic acid can be or can encode an RNA agent. The nucleic acid can comprise a promoter operably linked to the polynucleotide encoding a protein. As disclosed herein, the gene is operatively linked with appropriate regulatory elements in some embodiments. The one or more genes of the nucleic acid can comprise an siRNA, an shRNA, an antisense RNA oligonucleotide, an antisense miRNA, a trans-splicing RNA, a guide RNA, a single-guide RNA, a crRNA, a tracrRNA, a trans-splicing RNA, a pre-mRNA, an mRNA, or any combination thereof. The one or more genes of the nucleic acid can comprise one or more synthetic protein circuit components. The one or more genes of the nucleic acid can comprise can entire synthetic protein circuit comprising one or more synthetic protein circuit components. The one or more genes of the nucleic acid can comprise two or more synthetic protein circuits.

The protein can be any protein, including naturally-occurring and non-naturally occurring proteins. Examples include, but are not limited to, luciferases; fluorescent proteins (e.g., GFP); growth hormones (GHs) and variants thereof; insulin-like growth factors (IGFs) and variants thereof granulocyte colony-stimulating factors (G-CSFs) and variants thereof erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof clotting factors and variants thereof; cystic fibrosis transmembrane conductance regulator (CFTR) and variants thereof; and interferons and variants thereof.

Examples of protein of interest include, but are not limited to, luciferases; fluorescent proteins (e.g., GFP); growth hormones (GHs) and variants thereof; insulin-like growth factors (IGFs) and variants thereof granulocyte colony-stimulating factors (G-CSFs) and variants thereof erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; CFTR and variants thereof; and interferons and variants thereof.

In some embodiments, the protein of interest is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (GF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-α receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Groa/IL-S, RANTES, M1P-1 a, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-rel easing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of nietalloproteinases; vasoactive intestinal peptide; angiogenin; angiotropin; fibrin; hirudin; IL-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor IX or Factor X; dystrophin or nini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, f3-enolase, and glycogen synthase; lysosomal enzymes (e.g., beta-N-acetylhexosaminidase A); and any variants thereof.

The protein of interest can be, for example, an active fragment of a protein, such as any of the aforementioned proteins, a fusion protein comprising some or all of two or more proteins, or a fusion protein comprising all or a portion of any of the aforementioned proteins.

In some embodiments, the viral vector comprises a polynucleotide comprising coding regions for two or more proteins of interest, The two or more proteins of interest can be the same or different from each other. In some embodiments, the two or more proteins of interest are related polypeptides, for example light chain(s) and heavy chain(s) of the same antibody.

The protein of interest can be a multi-subunit protein. For example, the protein of interest can comprise two or more subunits, or two or more independent polypeptide chains. In some embodiments, the protein of interest can be an antibody, including, but not limited to, antibodies of various isotypes (for example, IgG1, IgG2, IgG3, IgG, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody. In some embodiments, the protein of interest is not an immune-adhesin.

In some embodiments, the resulting targeting molecules can be employed in methods and/or therapies relating to in vivo gene transfer applications to long-lived cell populations. In some embodiments, these can be applied to any rAAV-based gene therapy, including, for example: spinal muscular atrophy (SMA), amyotrophic lateral sclerosis (ALS), Parkinson's disease, Friedreich's ataxia, Pompe disease, Huntington's disease, Alzheimer's disease, Battens disease, lysosomal storage disorders, glioblastoma multiforme, Rett syndrome, Leber's congenital amaurosis, chronic pain, stroke, spinal cord injury, traumatic brain injury and lysosomal storage disorders. In addition, rAAVs can also be employed for in vivo delivery of transgenes for non-therapeutic scientific studies such as optogenetics, gene overexpression, gene knock-down with shRNA or miRNAs, modulation of endogenous miRNAs using miRNA sponges or decoys, recombinase delivery for conditional gene deletion, conditional (recombinase-dependent) expression, or gene editing with CRISPRs, TALENs, and zinc finger nucleases.

In some embodiments, the gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments of the nucleic acids provided herein can be employed as vaccines for the prophylaxis or treatment of infectious diseases (e.g., as vaccines).

As described herein, the nucleotide sequence encoding the protein can be modified to improve the expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of gene expression in the target cell can vary. The amount of the protein expressed in the subject (e.g., the CNS of the subject) can vary. For example, in some embodiments the protein can be expressed in the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the protein is expressed in the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a protein is needed for the method to be effective can vary depending on non-limiting factors such as the particular protein and the subject receiving the treatment, and an effective amount of the protein can be readily determined by a skilled artisan using conventional methods known in the art without undue experimentation.

The agent (e.g., the therapeutic agent) can be an inducer of cell death. The agent can induce cell death by a non-endogenous cell death pathway (e.g., a bacterial pore-forming toxin). In some embodiments, the agent (e.g., a protein encoded by a nucleic acid) can be a pro-survival protein. In some embodiments, the agent is a modulator of the immune system. The agent can activate an adaptive immune response, and innate immune response, or both. In some embodiments, the nucleic acid encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments of the compositions provided herein can be employed as vaccines for the prophylaxis or treatment of infectious diseases (e.g., as vaccines). The protein can comprise a CRE recombinase, GCaMP, a cell therapy component, a knock-down gene therapy component, a cell-surface exposed epitope, or any combination thereof. In some embodiments, the protein comprises CFTR, GDE, OTOF, DYSF, MYO7A, ABCA4, F8, CEP290, CDH23, DMD, and ALMS1.

The agent (e.g., the therapeutic agent) can comprise a non-protein coding gene, such as an RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some embodiments, the RNA agent can comprise non-natural or modified nucleotides (e.g., pseudouridine). In some embodiments, the non-protein coding gene can modulate the expression or the activity of a target gene or gene expression product. For example, the RNAs described herein may be used to inhibit gene expression in a target cell, for example, a cell in the central nervous system (CNS). In some embodiments, inhibition of gene expression refers to inhibition by at least about 20%, 30%, 40%, 50%, 60%, 70%, 80%, 85%, 90%, 95% or 100%. In some cases, the protein product of the targeted gene is inhibited by at least about 20%, 30%, 40%, 50%, 60%, 70%, 80%, 85%, 90%, 95% or 99%. The gene can be either a wild type gene or a gene with at least one mutation. The targeted protein can be a wild type protein, or a protein with at least one mutation.

Examples of genes encoding therapeutic proteins include a sequence associated with a signaling biochemical pathway, e.g., a signaling biochemical pathway-associated gene or polynucleotide (e.g., a signal transducer). In some embodiments, the methods and compositions disclosed herein comprise knockdown of an endogenous signal transducer accompanied by tuned expression of a protein comprising an appropriate version of signal transducer. Examples of DNA or RNA sequences contemplated herein include sequences for a disease-associated gene or polynucleotide. A “disease-associated” gene or polynucleotide refers to any gene or polynucleotide which is yielding transcription or translation products at an abnormal level or in an abnormal form in cells derived from disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products can be known or unknown, and can be at a normal or abnormal level. Signal transducers can be associated with one or more diseases or disorders. In some embodiments, a disease or disorder is characterized by aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder.

Many proteins (e.g., enzymes) are secreted and can exert cross-correction effects. For these, the genetic material can be delivered to brain endothelial cells using an AAV of the present disclosure, transforming these cells into a biofactory to produce and distribute therapeutics to other cell types. For example, the production of the secreted Sparcl1/Hevin protein in brain endothelial cells can rescue the thalamocortical synapse loss phenotype of Hevin KO mice. This proof-of-concept supports the brain endothelial cell biofactory model for the production of enzymes, antibodies, or other biological therapeutics, providing a novel therapeutic approach for diseases like lysosomal storage disorders.

In some embodiments, the rAAV having a capsid protein comprising one or more targeting peptides disclosed herein can be used to deliver genes to specific cell types in the target environment of a subject. For example, the rAAV can be used for delivering genes to neurons and glia in the nervous system (including PNS, CNS, or both) of a subject (e.g., a mammal). The compositions and methods disclosed herein can be used in, for example, (i) reducing the expression of mutant Huntingtin in patients with Huntington's Disease by, for example, incorporating a Huntingtin-specific microRNA expression cassette within a rAAV genome and packaging the rAAV genome into a variant rAAV for delivery through, for example the vasculature, (ii) delivering a functional copy of the Frataxin gene to patients with Friedreich's ataxia, (iii) restoring expression of an enzyme critical for normal lysosomal function in patients lacking expression of the enzyme due to genetic mutation (e.g., patients with Neimann-Pick disease, mucopolysaccharidosis III, and/or Gaucher's disease), (iv) using the rAAV to generate animal models of disease, or a combination thereof.

The subject in need can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

In some embodiments, the subject is suffering from an acute condition or injury. The subject in need can be a subject suffering from, at risk to develop, or has suffered from a stroke, traumatic brain injury, epilepsy, or spinal cord injury.

### Pharmaceutical Compositions and Methods of Administration

Also disclosed herein are pharmaceutical compositions comprising one or more of the rAAV viruses (or other delivery systems) disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Disclosed herein include methods of delivering an agent to a nervous system of a subject. In some embodiments, the method comprises: providing an AAV vector comprising an AAV capsid protein disclosed herein. In some embodiments, the AAV vector comprises an agent to be delivered to the nervous system. In some embodiments, the method comprises administering the AAV vector to the subject. The composition can be for intravenous administration. The composition can be for systemic administration. The agent can be delivered to endothelial lining of the ventricles in the brain, the central canal of the spinal cord, capillaries in the brain, arterioles in the brain, arteries in the brain, or a combination thereof of the subject. The subject can be an adult animal.

Titers of the rAAV to be administered will vary depending, for example, on the particular rAAV, the mode of administration, the treatment goal, the individual, and the cell type(s) being targeted, and can be determined by methods standard in the art. As will be readily apparent to one skilled in the art, the useful in vivo dosage of the recombinant virus to be administered and the particular mode of administration will vary depending upon the age, weight, severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

The exact dosage can be determined on a drug-by-drug basis, in most cases, some generalizations regarding the dosage can be made. In some embodiments, the rAAV for delivery of an agent to the nervous system (e.g., CNS) of a subject can be administered, for example via injection, to a subject at a dose of between 1×10¹⁰ viral genome (vg) of the recombinant virus per kg of the subject and 2×10¹⁴ vg per kg, for example between 5×10¹¹ vg/kg and 5×10¹² vg/kg. In some embodiments, the dose of the rAAV administered to the subject is no more than 2×10¹⁴ vg per kg. In some embodiments, the dose of the rAAV administered to the subject is no more than 5×10¹² vg per kg. In some embodiments, the dose of the rAAV administered to the subject is no more than 5×10¹¹ vg per kg.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g., individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some embodiments, pharmaceutical compositions in accordance with the present disclosure are administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations can be converted to vg or viral genomes per kg or into total viral genomes administered by one of skill in the art.

In some embodiments, a dose of the pharmaceutical composition comprises a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases the concentration of infectious particles is 2×10⁷, 2×10⁸, 2×10⁹, 2×10¹⁰, 2×10⁻¹¹, 2×10¹², 2×10¹³, 2×10¹⁴, 2×10¹⁵, 2×10¹⁶, 2×10¹⁷, or a range between any two of these values. In some cases the concentration of the infectious particles is 3×10⁷, 3×10⁸, 3×10⁹, 3×10¹⁰, 3×10⁻¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, 4×10¹⁷, or a range between any two of these values. In some cases the concentration of the infectious particles is 5×10⁷, 5×10⁸, 5×10⁹, 5×10¹⁰, 5×10⁻¹¹, 5×10¹², 5×10¹³, 5×10¹⁴, 5×10¹⁵, 5×10¹⁶, 5×10¹⁷, or a range between any two of these values. In some cases the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases the concentration of the infectious particles is 7×10⁷, 7×10⁸, 7×10⁹, 7×10¹⁰, 7×10⁻¹¹, 7×10¹², 7×10¹³, 7×10¹⁴, 7×10¹⁵, 7×10¹⁶, 7×10¹⁷, or a range between any two of these values. In some cases the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

The recombinant viruses disclosed herein can be administered to a subject (e.g., a human) in need thereof. The route of the administration is not particularly limited. For example, a therapeutically effective amount of the recombinant viruses can be administered to the subject by via routes standard in the art. The administration can be a systemic administration. The administration can be an intravenous administration.

Non-limiting examples of the route include intramuscular, intravaginal, intravenous, intraperitoneal, subcutaneous, epicutaneous, intradermal, rectal, intraocular, pulmonary, intracranial, intraosseous, oral, buccal, systematic, or nasal. In some embodiments, the recombinant virus is administered to the subject by systematic transduction. In some embodiments, the recombinant virus is administered to the subject by intramuscular injection. In some embodiments, the rAAV is administered to the subject by the parenteral route (e.g., by intravenous, intramuscular or subcutaneous injection), by surface scarification or by inoculation into a body cavity of the subject. Route(s) of administration and serotype(s) of AAV components of the rAAV virus can be readily determined by one skilled in the art taking into account the infection and/or disease state being treated and the target cells/tissue(s) that are to express the protein of interest. In some embodiments, it can be advantageous to administer the rAAV via intravenous administration. The variant AAV provided herein can advantageously provide for intravenous administration of vectors with enhanced tropisms for CNS.

In some embodiments, the subject is a primate and the agent is delivered to the endothelial cells and/or neurons of the nervous system. The nervous system can be the central nervous system (CNS). The agent can be delivered to the endothelial cells of the nervous system of the subject at least 1.5-fold, 2-fold, or 3-fold more efficiently than the delivery of the agent to the neurons of the nervous system. In some embodiments, the agent is delivered to the endothelial cells of the nervous system of the subject more than 3-fold more efficiently (e.g., 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to the neurons of the nervous system.

Disclosed herein include methods of delivering an agent (e.g., a therapeutic agent) to a cell. In some embodiments, the method comprises: contacting an AAV vector comprising an AAV capsid protein disclosed herein with the cell. In some embodiments, the AAV vector comprises an agent to be delivered to the nervous system. In some embodiments, the cell is an endothelial cell or a neuron. In some embodiments, contacting the AAV vector with the cell occurs in vitro, in vivo or ex vivo. The cell can be present in a tissue, an organ, or a subject. The cell can be a brain endothelial cell, a neuron, a cell in the capillaries in the brain, a cell in the arterioles in the brain, a cell in the arteries in the brain, a cell in the brain vasculature, or a combination thereof.

The AAV vector can be an AAV9 vector, or a variant thereof. In some embodiments, the AAV vector is a vector selected from AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. The serotype of the AAV vector can be different from the serotype of the AAV capsid.

The variant AAV capsid can comprise tropism for a tissue or a cell of a central nervous system (CNS). The target cell can be a neuronal cell, a neural stem cell, an astrocyte, or a tumor cell. The target cell can be located in a brain or spinal cord. The target cell can comprise an antigen-presenting cell, a dendritic cell, a macrophage, a neural cell, a brain cell, an astrocyte, a microglial cell, and a neuron. In some embodiments, the target cell is an endothelial cell.

Actual administration of the rAAV can be accomplished by using any physical method that will transport the rAAV into the nervous system of the subject. For example, the rAAV disclosed herein can advantageously be administered intravenously for delivery to the CNS. As disclosed herein, capsid proteins of the rAAV can be modified so that the rAAV is targeted to a particular target environment of interest such as central nervous system, and to enhance tropism to the target environment of interest (e.g., CNS tropism). Pharmaceutical compositions can be prepared, for example, as injectable formulations.

The recombinant virus to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of the recombinant virus expressing the therapeutic protein is administered to a host in need of such treatment. The use of the recombinant virus disclosed herein in the manufacture of a medicament for inducing immunity in, or providing gene therapy to, a host is within the scope of the present application.

In instances where human dosages for the rAAV have been established for at least one condition, those same dosages, or dosages that are between about 0.1% and 500%, more preferably between about 25% and 250% of the established human dosage can be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

A therapeutically effective amount of the rAAV can be administered to a subject at various points of time. For example, the rAAV can be administered to the subject prior to, during, or after the subject has developed a disease or disorder. The rAAV can also be administered to the subject prior to, during, or after the occurrence of a disease or disorder (e.g., Huntington's disease (HD), Alzheimer's disease, Parkinson's disease, Amyotrophic lateral sclerosis, spinal muscular atrophy, types I and II, Friedreich's Ataxia, Spinocerebellar ataxia and any of the lysosomal storage disorders that involve cells with CNS, which includes but is not limited to Krabbe disease, Sandhoff disease, Tay-Sachs, Gaucher disease (Type I, II, or 111), Niemann-Pick disease (NPC1 or NPC2 deficiency), Hurler syndrome, Pompe disease, Batten disease, or any combination thereof), chronic pain, or a combination thereof. In some embodiments, the rAAV is administered to the subject during remission of the disease or disorder. In some embodiments, the rAAV is administered prior to the onset of the disease or disorder in the subject. In some embodiments, the rAAV is administered to a subject at a risk of developing the disease or disorder.

The disease or disorder can comprise a neurological disease or disorder. For example, the neurological disease or disorder can comprise epilepsy, Dravet Syndrome, Lennox Gastaut Syndrome, myocolonic seizures, juvenile myocolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving f3-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

Disclosed herein, in some embodiments, are formulations of pharmaceutically-acceptable excipients and carrier solutions suitable for delivery of the compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some instances, the compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. In some embodiments, the rAAV disclosed herein can advantageously be administered intravenously for delivery to the CNS.

In some embodiments, the pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the compositions disclosed herein (e.g., rAAV compositions), which are prepared by incorporating the compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Formulations for intranasal administration can comprise a coarse powder comprising the active ingredient and having an average particle size from about 0.2 μm to 500 μm. Such formulations are administered in the manner in which snuff is taken, e.g., by rapid inhalation through the nasal passage from a container of the powder held close to the nose. Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, comprise 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions are within the purview of the skilled artisan having benefit of the present teachings. In some embodiments, however, that the administration of therapeutically-effective amounts of the disclosed compositions may be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In some embodiments, it is advantageous to provide multiple, or successive administrations of the AAV vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, infectious particles/ml given either as a single dose, or divided into two or more administrations as may be required to achieve therapy of the particular disease or disorder being treated. In fact, in some embodiments, it is desirable to administer two or more different AAV vector compositions, either alone, or in combination with one or more other therapeutic drugs to achieve the desired effects of a particular therapy regimen. In various embodiments, the daily and unit dosages are altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

The targeting peptides described herein can be used to generate rAAVs with enhanced CNS tropisms with capsid proteins derived from different AAV serotypes (e.g., AAV9 and AAV1). In some embodiments, this can advantageously provide for administration of two or more different AAV vector compositions without inducing immune response in the subject.

The dosing frequency of the rAAV virus can vary. For example, the rAAV virus can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the rAAV virus is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

Disclosed herein are kits comprising compositions disclosed herein. Also disclosed herein are kits for the treatment or prevention of a disease or conditions of the CNS, PNS, or target organ or environment (e.g., CNS). In some instances, the disease or condition is cancer, a pathogen infection, neurological disease, muscular disease, or an immune disorder, such as those described herein. In one embodiment, a kit can include a therapeutic or prophylactic composition containing an effective amount of a composition of a rAAV particle encapsidating a nucleic acid provided herein and a rAAV capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, rAAV are provided together with instructions for administering the rAAV to a subject having or at risk of developing the disease or condition. Instructions can generally include information about the use of the composition for the treatment or prevention of the disease or condition.

The kit can include allogenic cells. In some embodiments, a kit includes cells that can comprise a genomic modification. In some embodiments, a kit comprises “off-the-shelf” cells. In some embodiments, a kit includes cells that can be expanded for clinical use. In some embodiments, a kit contains contents for a research purpose.

In some embodiments, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some embodiments, instructions provide procedures for administering the rAAV to the subject alone. In some embodiments, instructions provide procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. In some instances, the instructions provide that the rAAV is formulated for intravenous injection. In some instances, the instructions provide that the rAAV is formulated for intranasal administration.

### EXAMPLES

Some aspects of the embodiments discussed above are disclosed in further detail in the following examples, which are not in any way intended to limit the scope of the present disclosure.

### Example 1

### Targets for Receptor-Mediated Control of Therapeutic Biodistribution and Efficacy

Described in this Example is a screen for identifying binding partners that mediate tropisms of engineered AAVs.

A panel of 6 AAVs including AAV9, AAV.CAP-Mac (aka CAP-C1), AAV.CAP-B22, AAV-X1.1 (aka X1B10), AAV.MaCPNS1 (aka PNS1), AAV.MaCPNS2 (aka PNS2) were added to fixed HEK293 cells expressing duplicate 6019 human plasma membrane proteins, secreted and cell surface tethered proteins as well as 397 human heterodimers. AAVs were pooled such that 3×10⁴ particles/cell of each of AAV9, AAV.CAP-Mac, AAV.CAP-B22, AAV.MaCPNS1, AAV.MaCPNS2, as well as 1×10⁴ particles/cell of AAV-X1.1 were applied. Following AAV addition, samples were directly fixed. Receptor-bound AAVs were labeled with an anti-AAV9 antibody and then a secondary detection antibody.

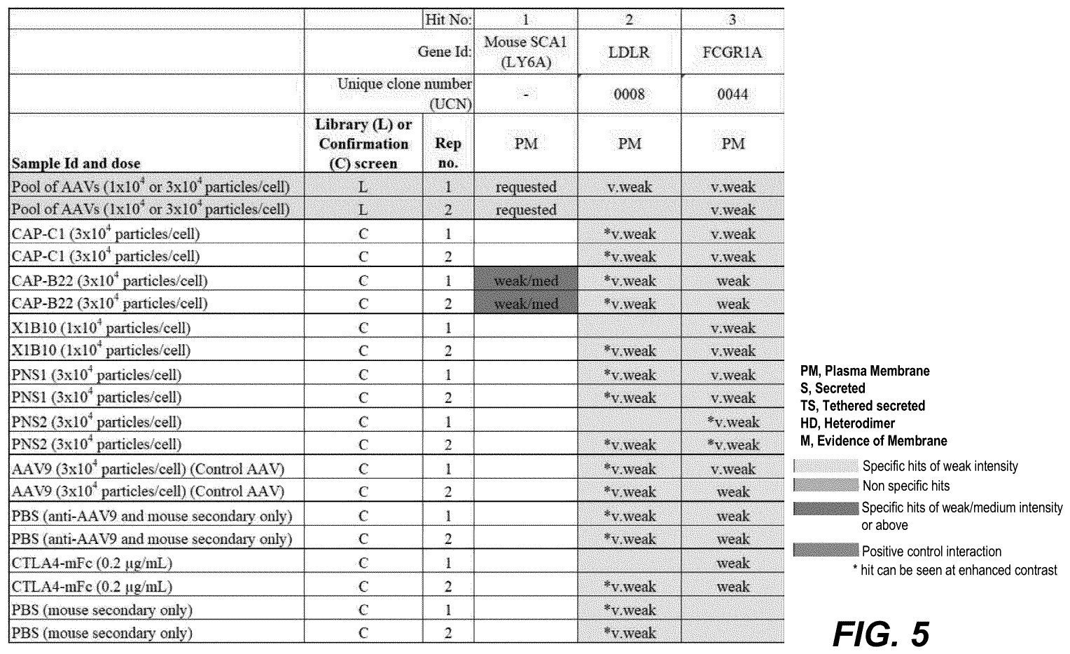

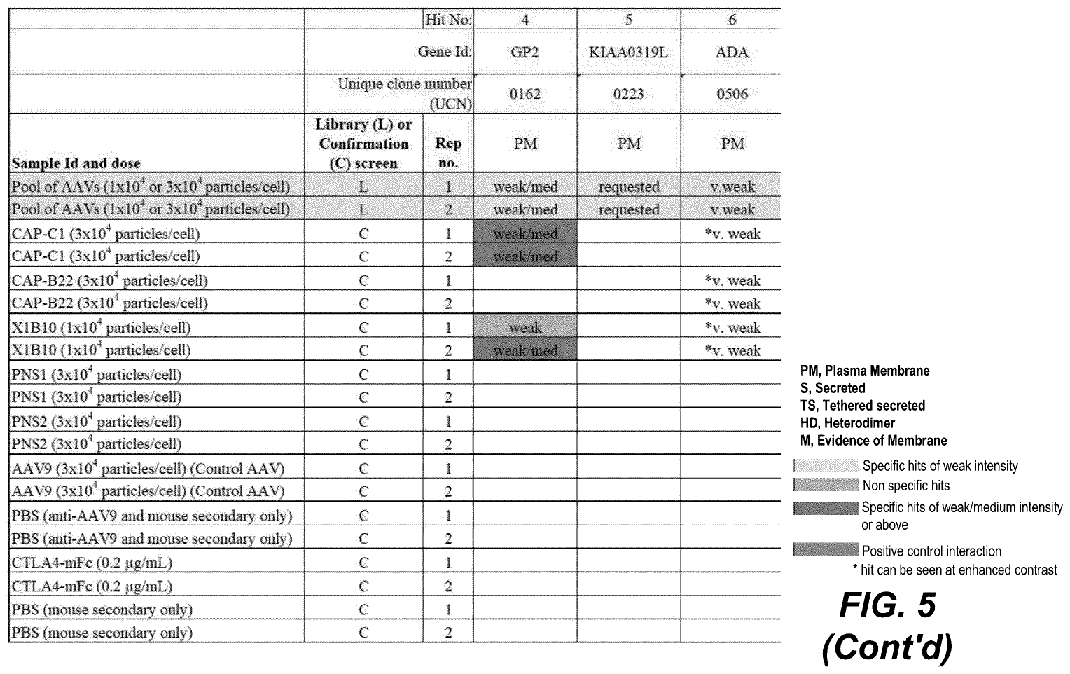

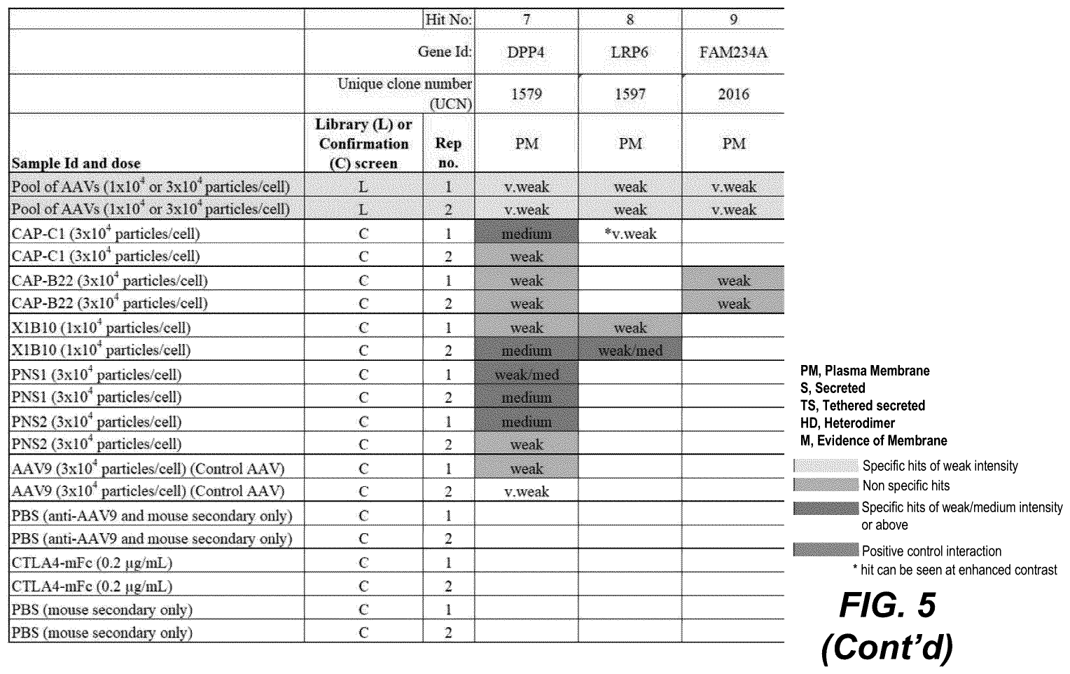

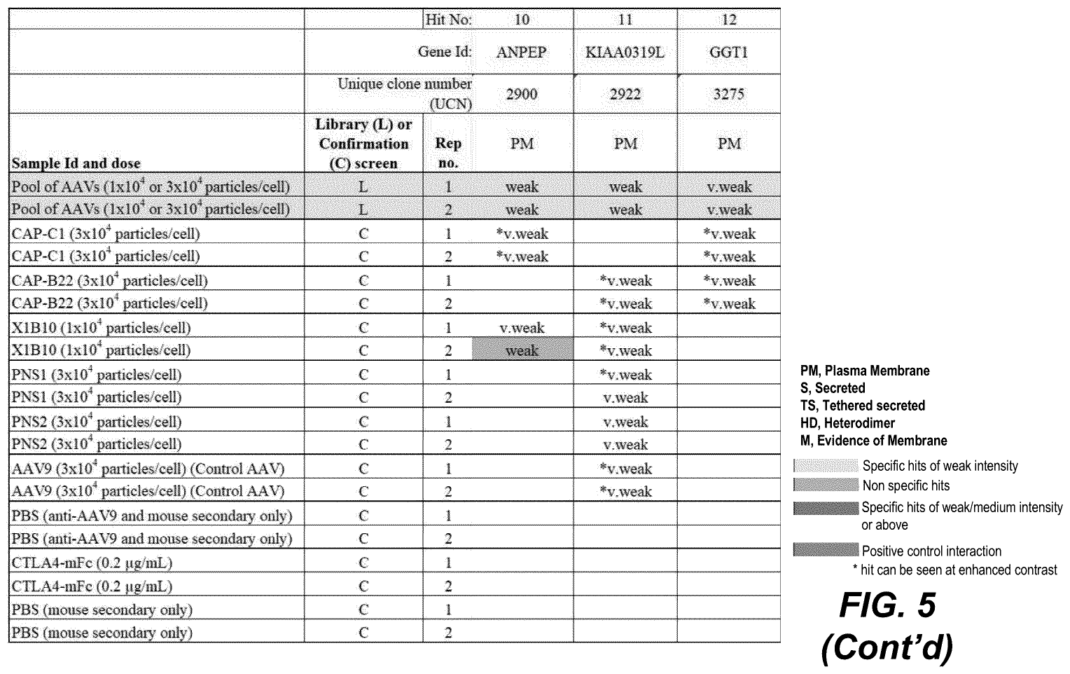

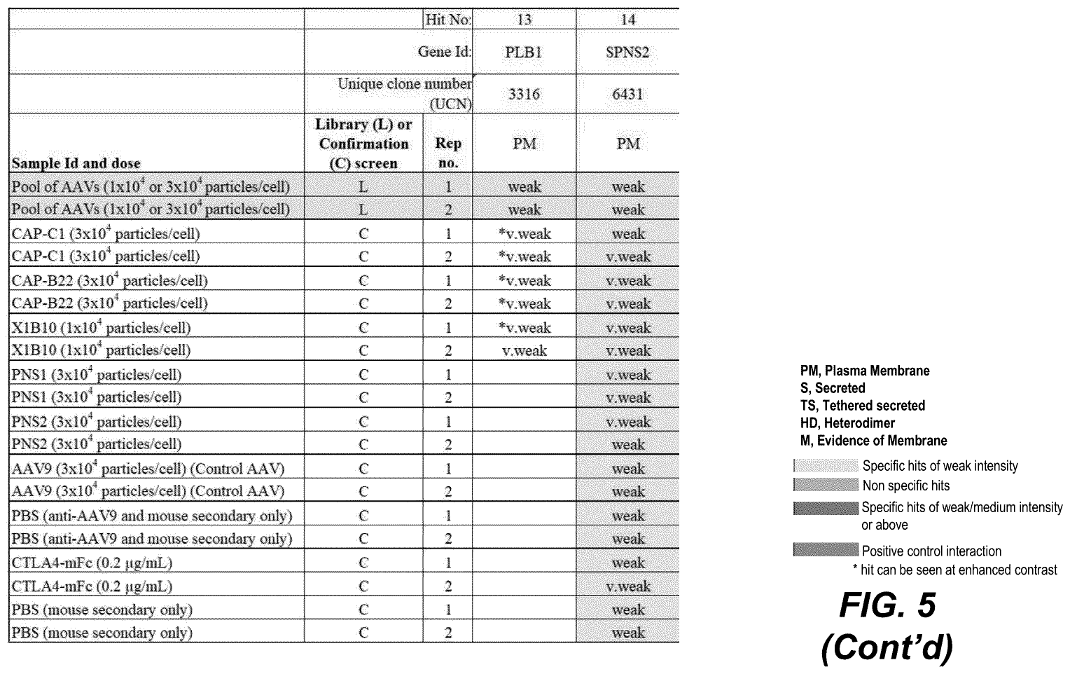

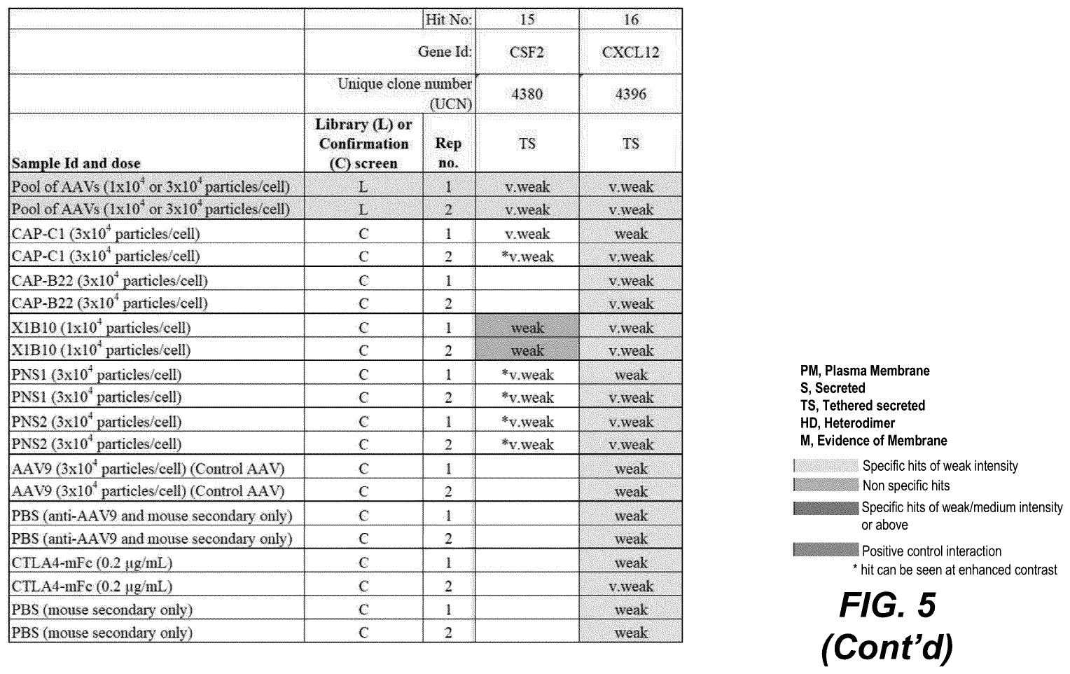

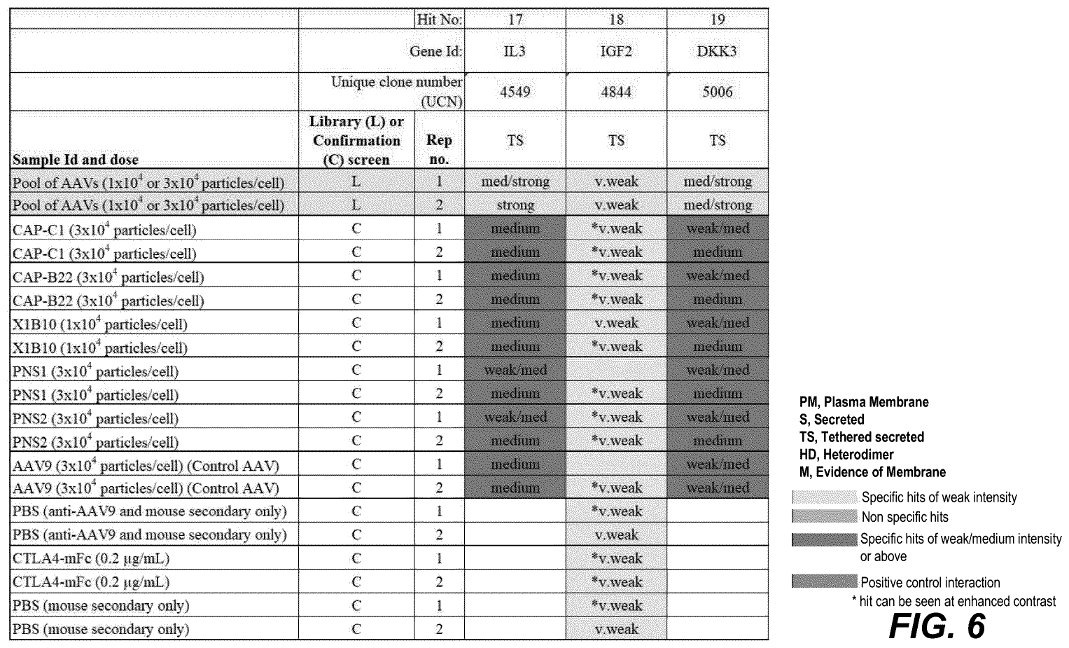

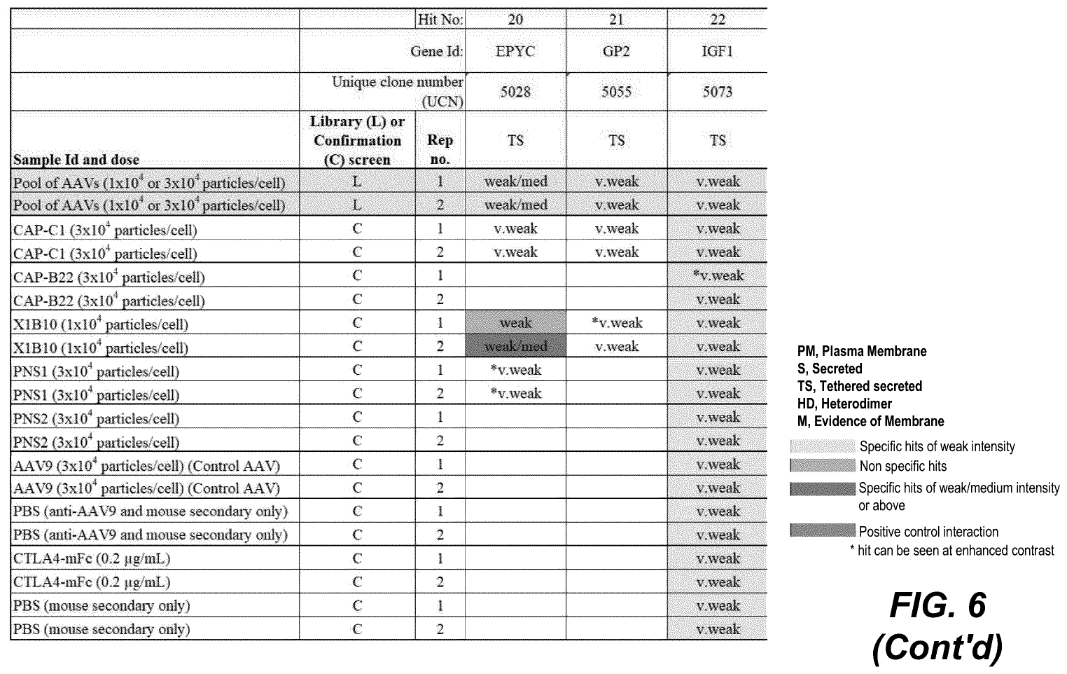

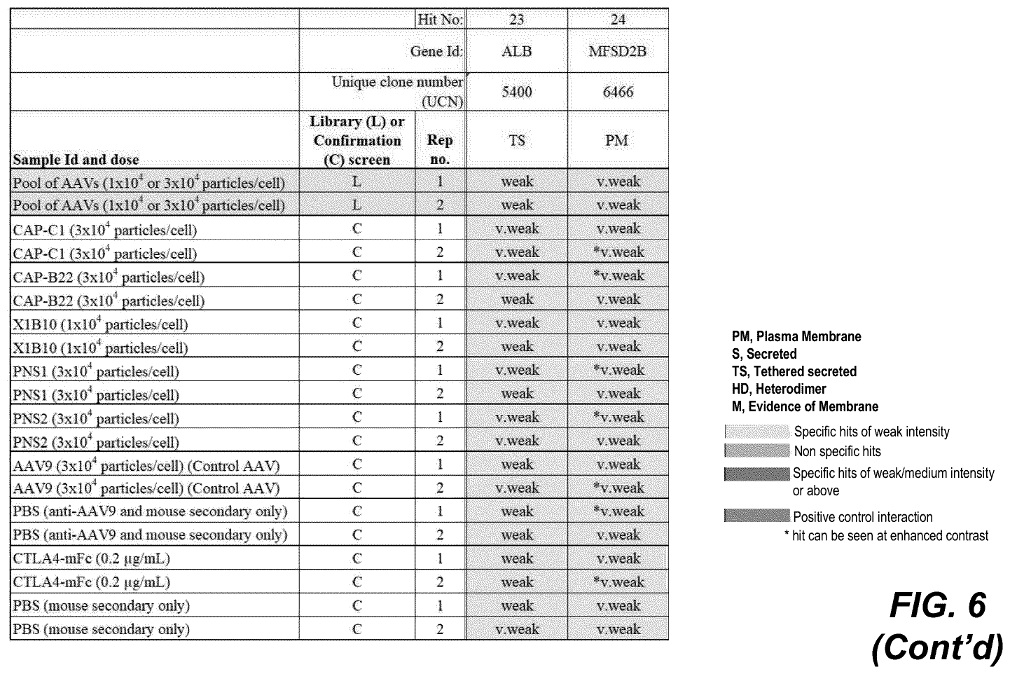

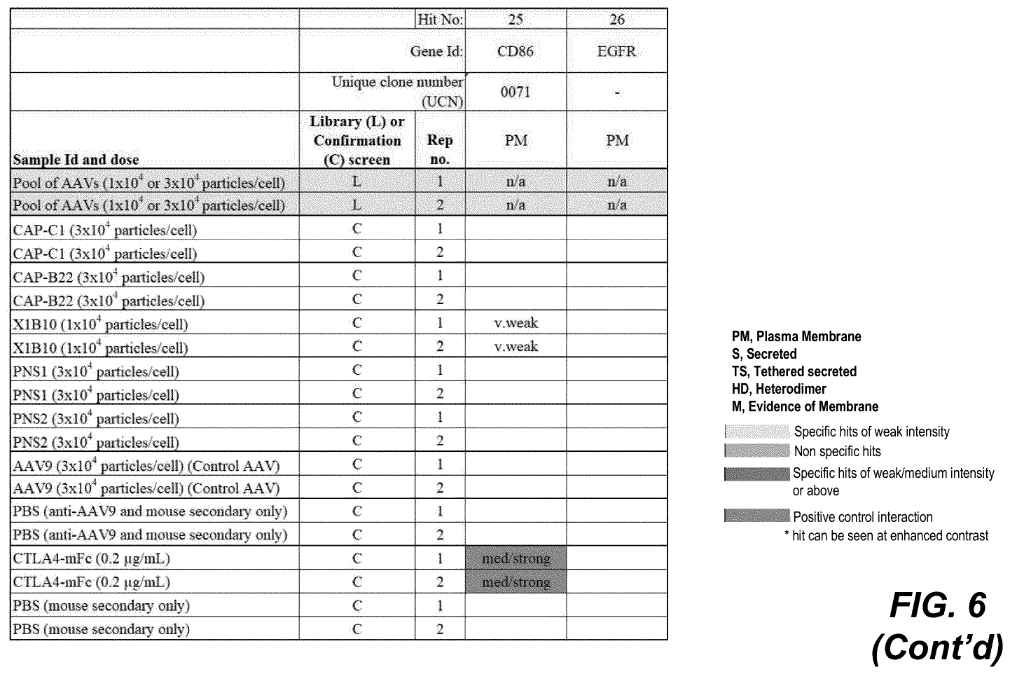

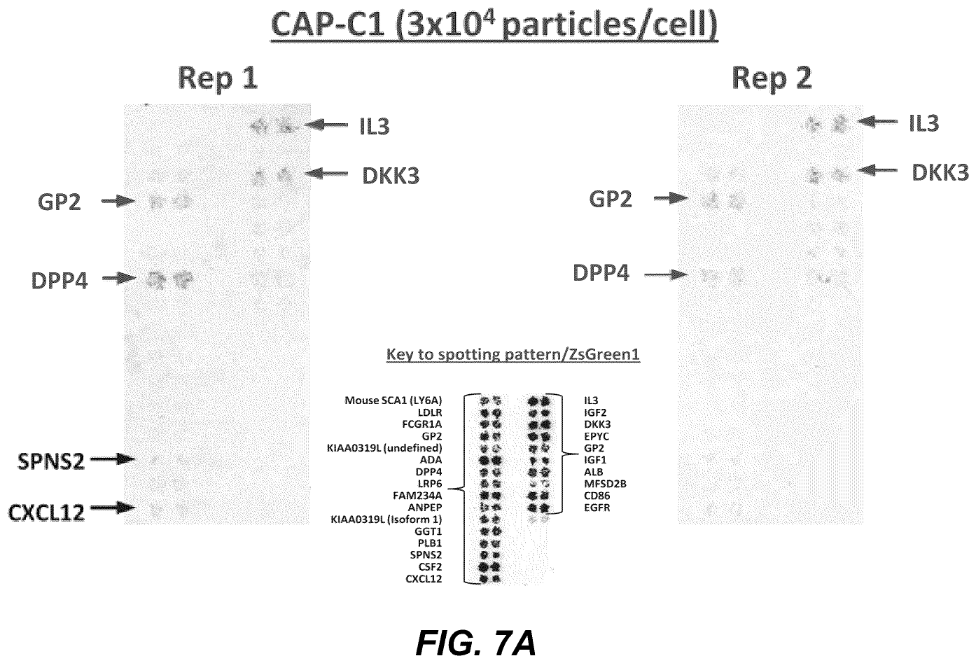

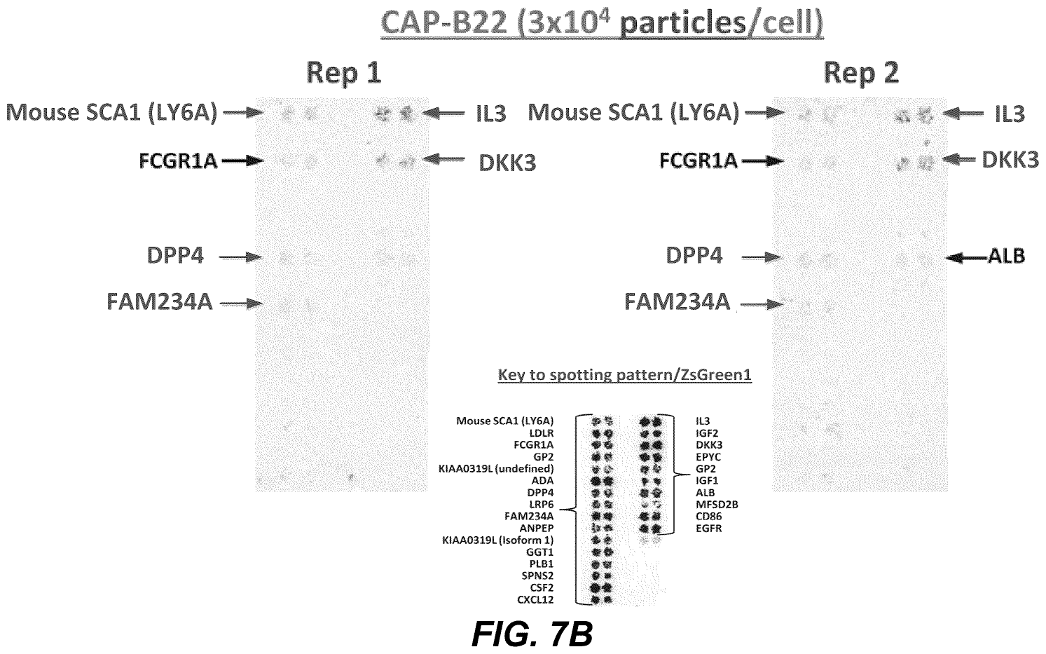

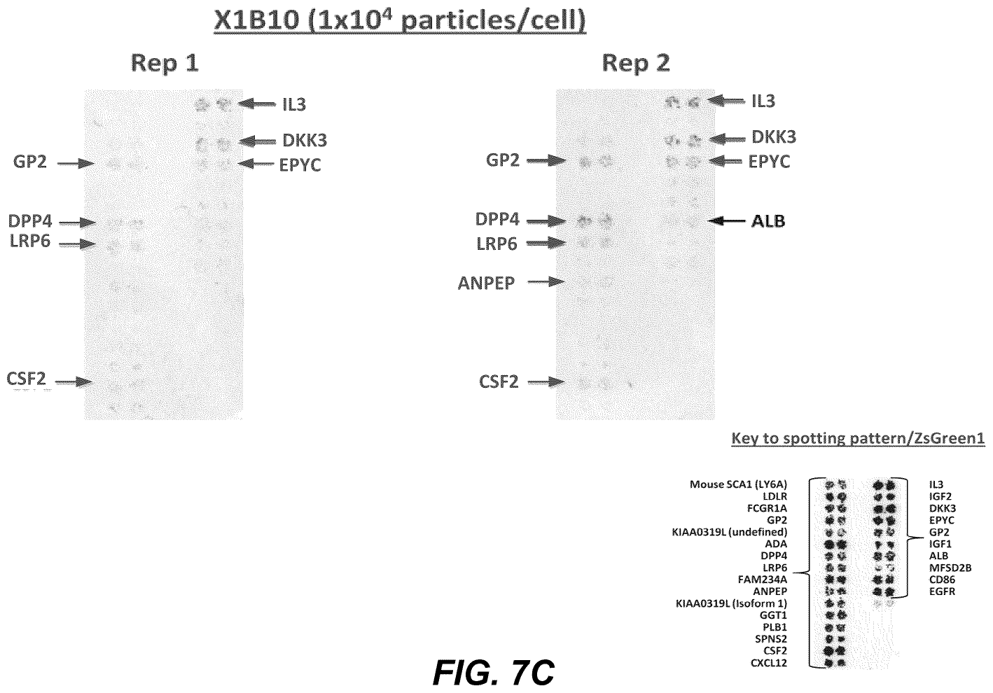

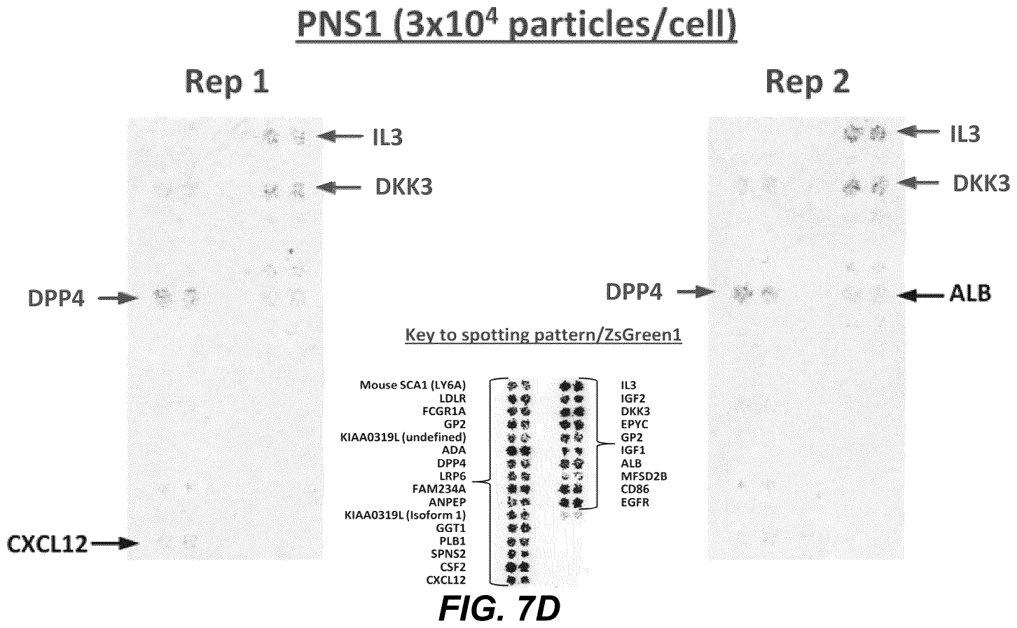

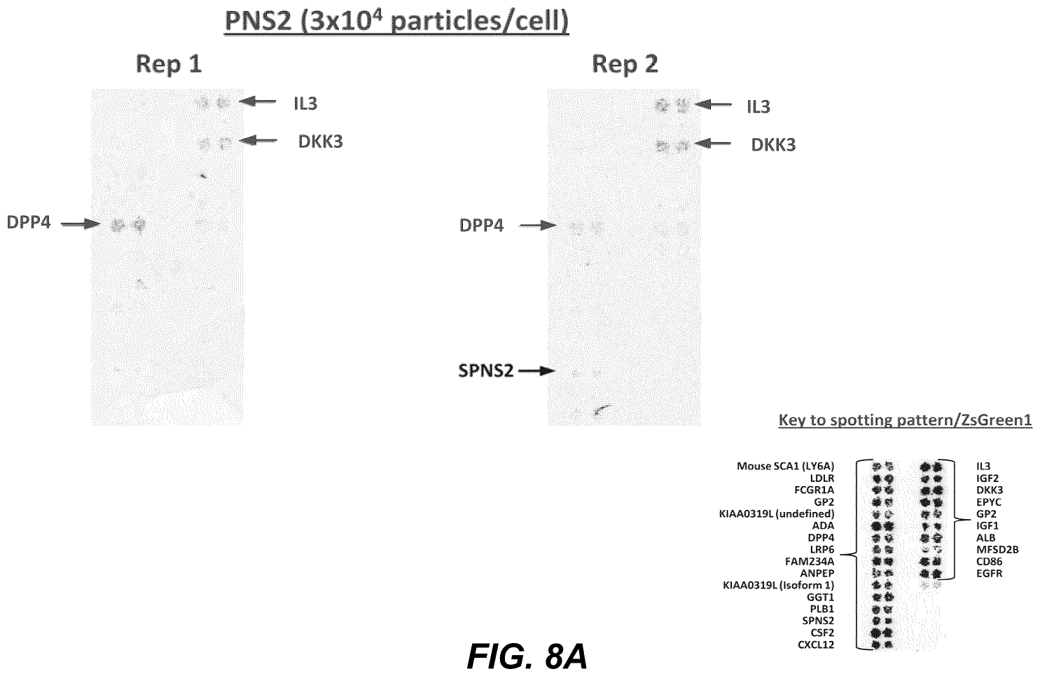

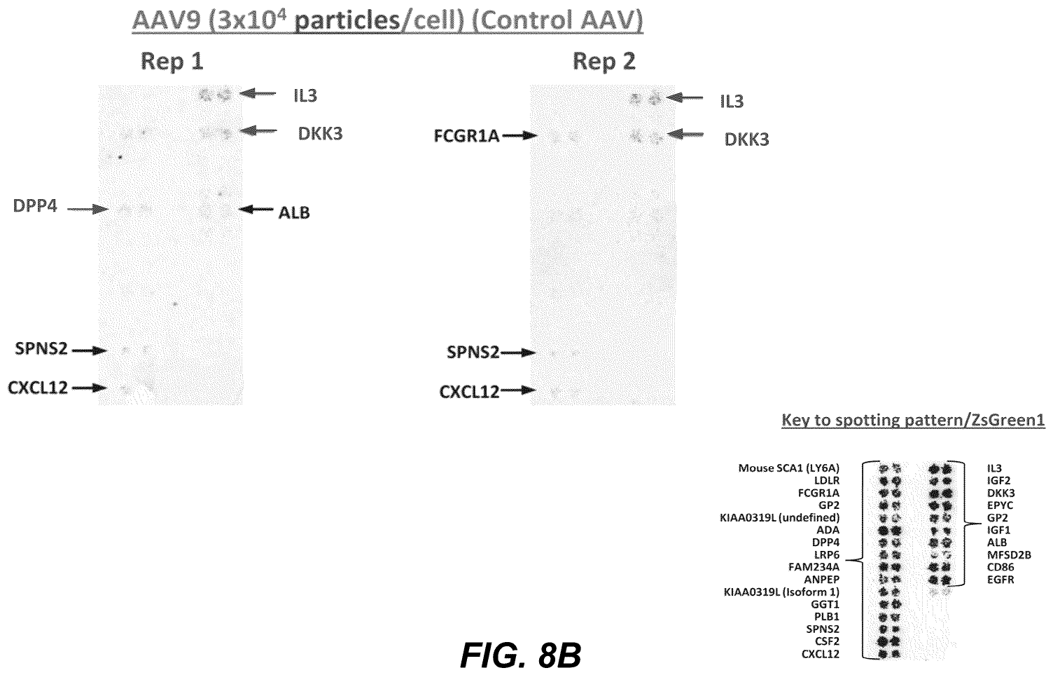

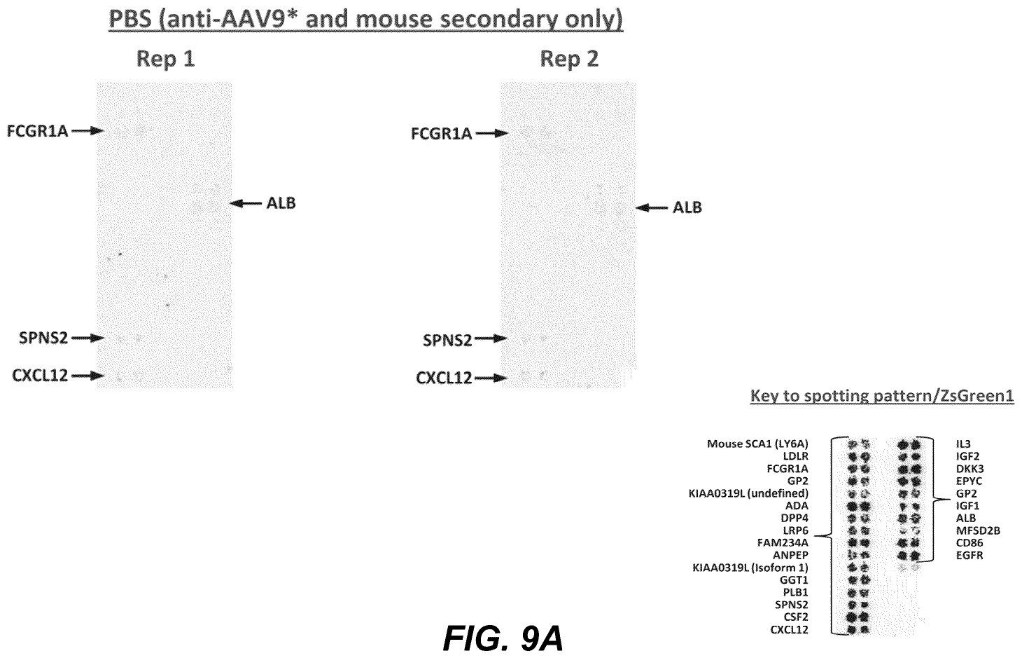

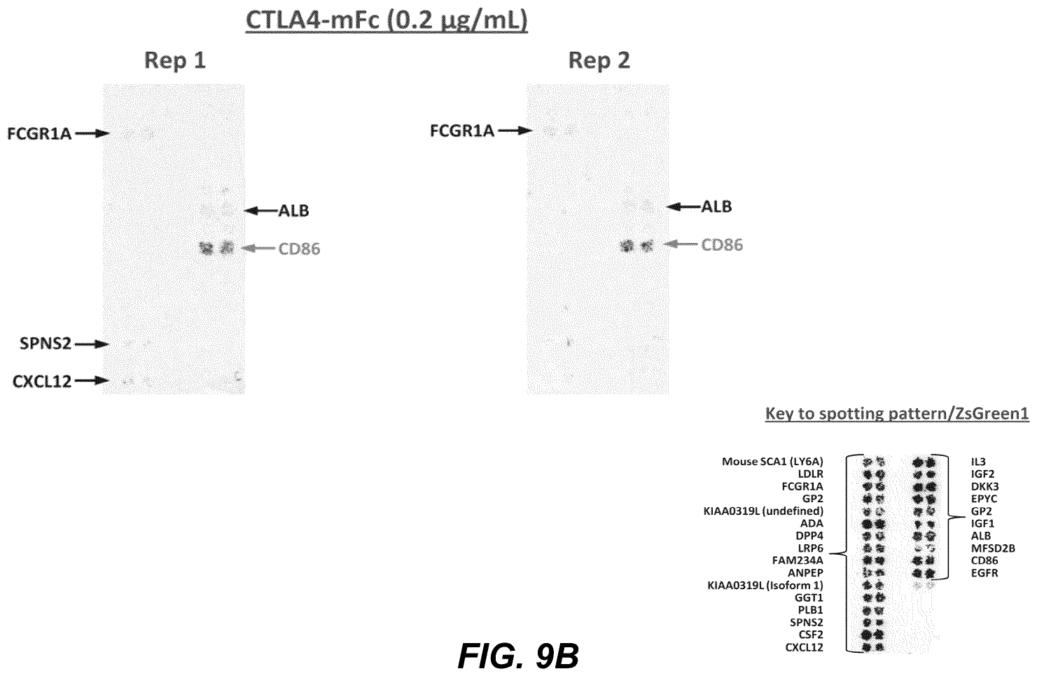

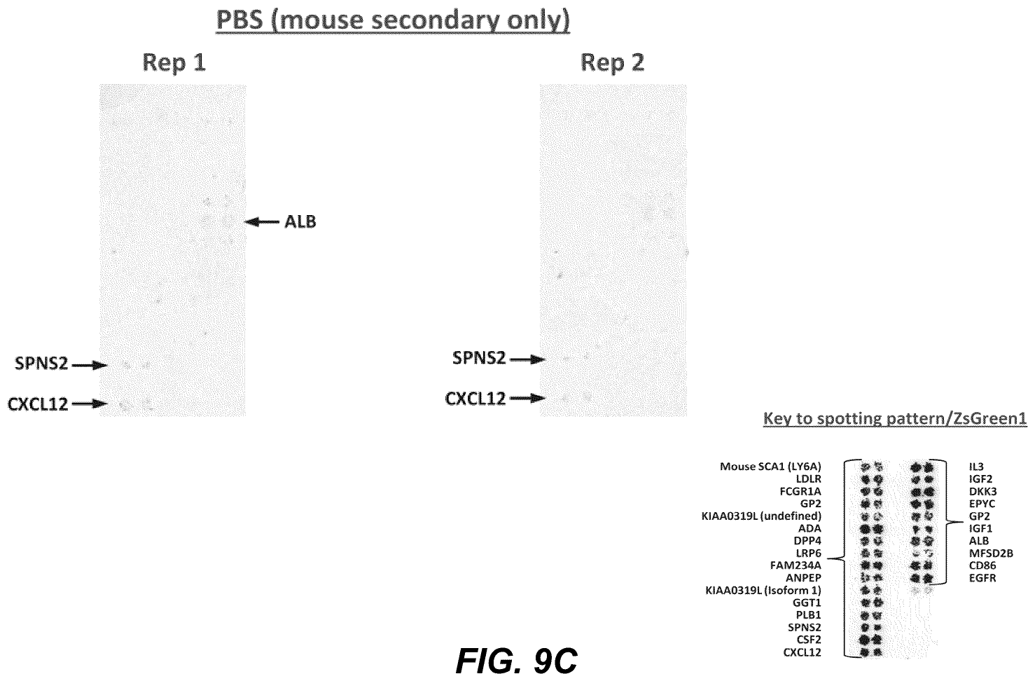

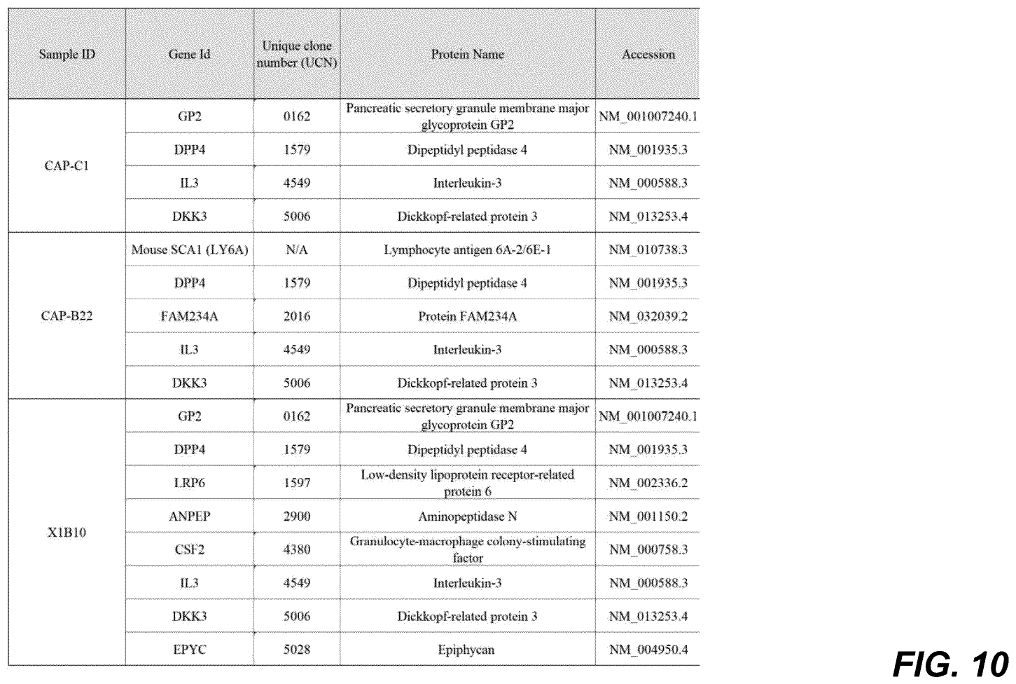

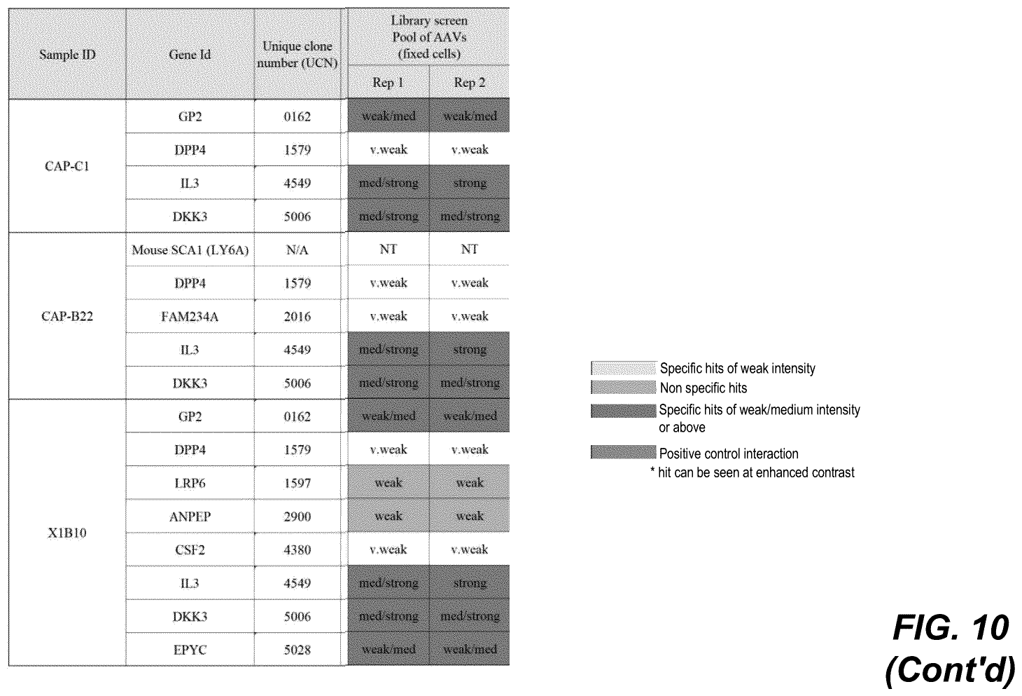

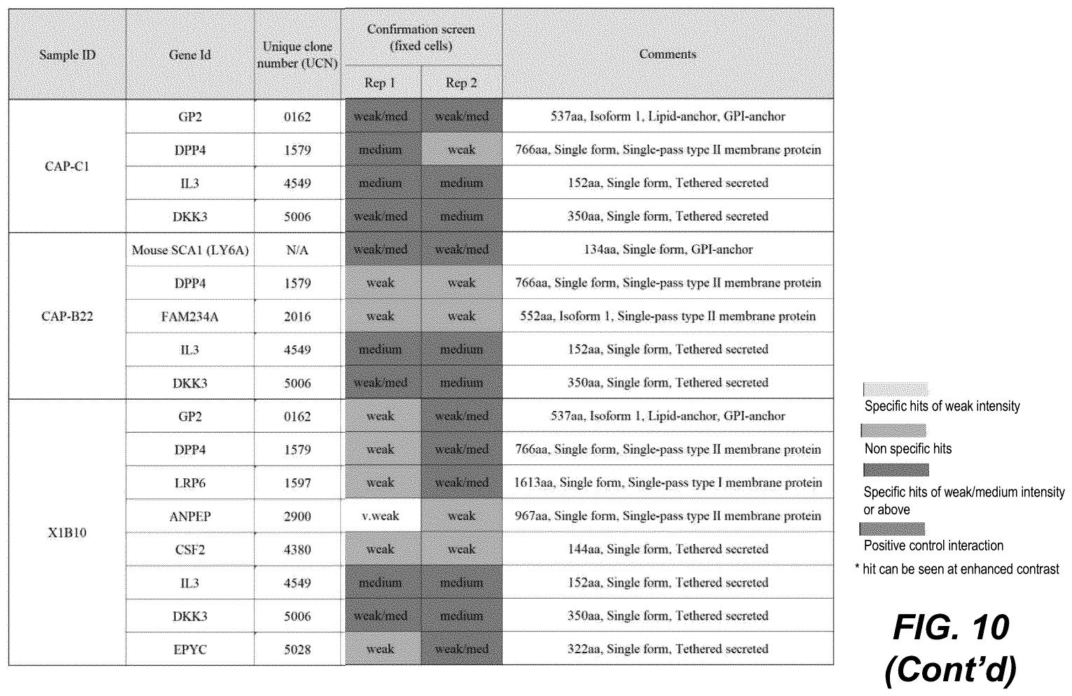

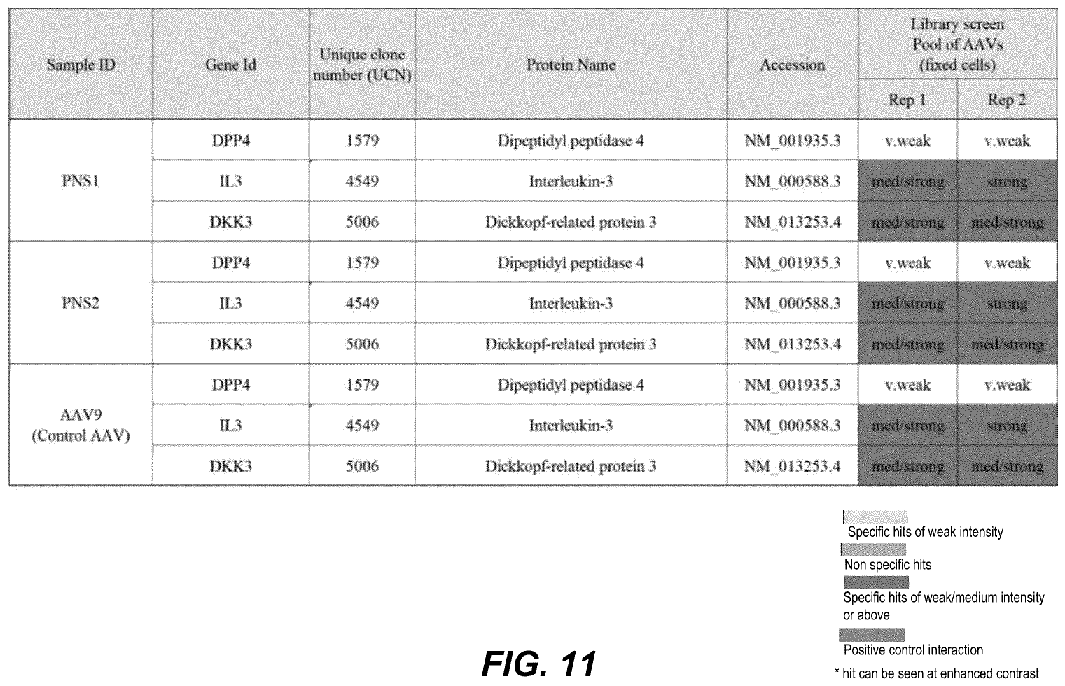

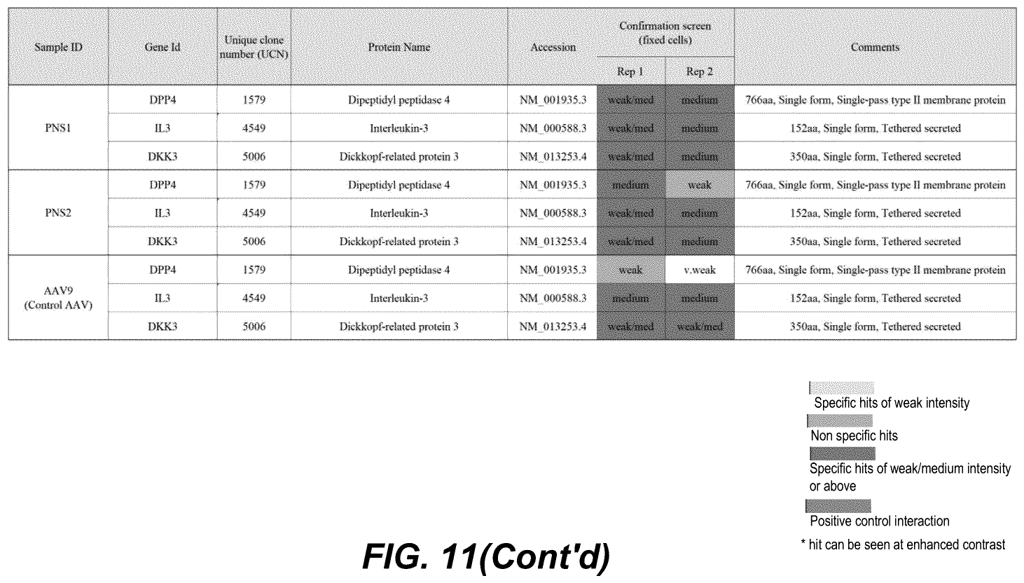

Following this, 22 library hits were identified and probed with each AAV individually in a confirmation screen that also included various positive and negative controls. The results of this screen are shown in FIG. 5-FIG. 11.

Three new targets were identified for AAV9: Dipeptidyl peptidase 4 (DPP4), interleukin-3 (IL-3), and Dickkopf-related protein 3 (DKK3). Without being bound by any particular theory, IL-3's role in T-cell activation and immune response to pathogens provides a means by which to modulate AAV's notably tolerant host immune response. Without being bound by any particular theory, DKK3's role as a WNT signaling inhibitor offers ways to modulate shuttling activity of DKK3 to WNT expressing cells and tissues. Binding of endogenous secreted factors like IL-3 and DKK3 may also function to cloak the viral particle in ‘self’ to minimize immune responses.

A new protein target for AAV.CAP-B22 is FAM234A. New targets that are specific for AAV-X1.1 include: pancreatic secretory granule membrane major glycoprotein GP2 (GP2), low-density lipoprotein receptor-related protein 6 (LRP6), aminopeptidase N (ANPEP), granulocyte-macrophage colony-stimulating factor (CSF2) and epiphycan (EPYC). These proteins collectively contribute to AAV-X1.1's altered tropism with respect to AAV9.

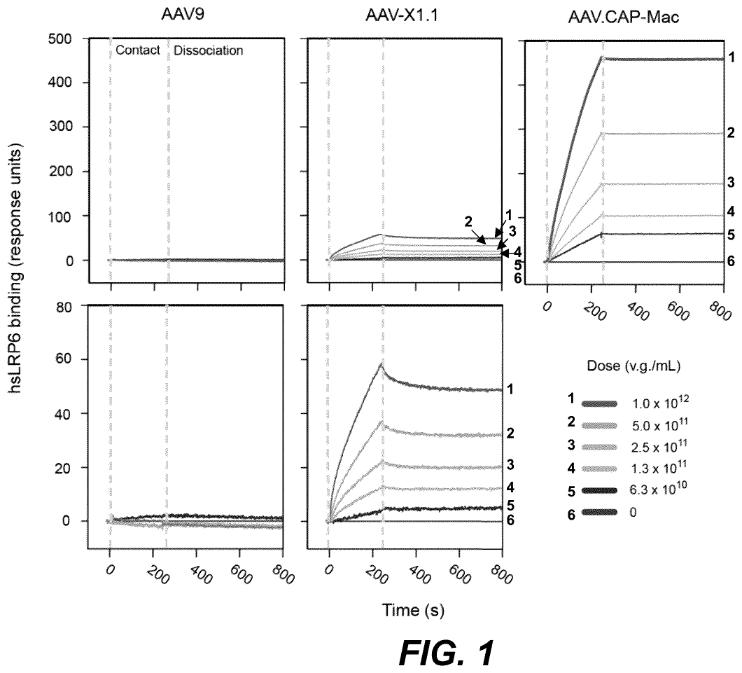

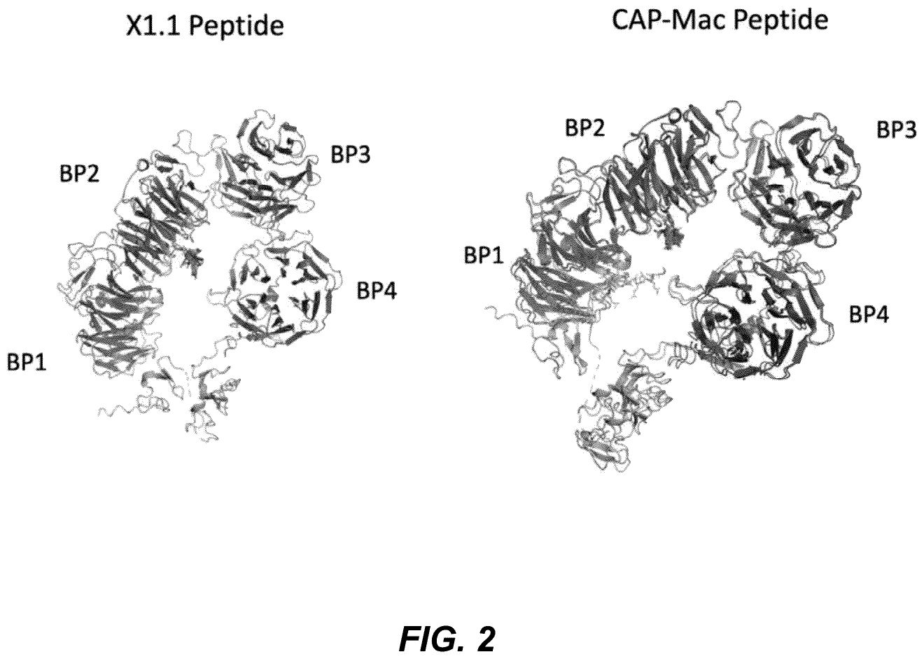

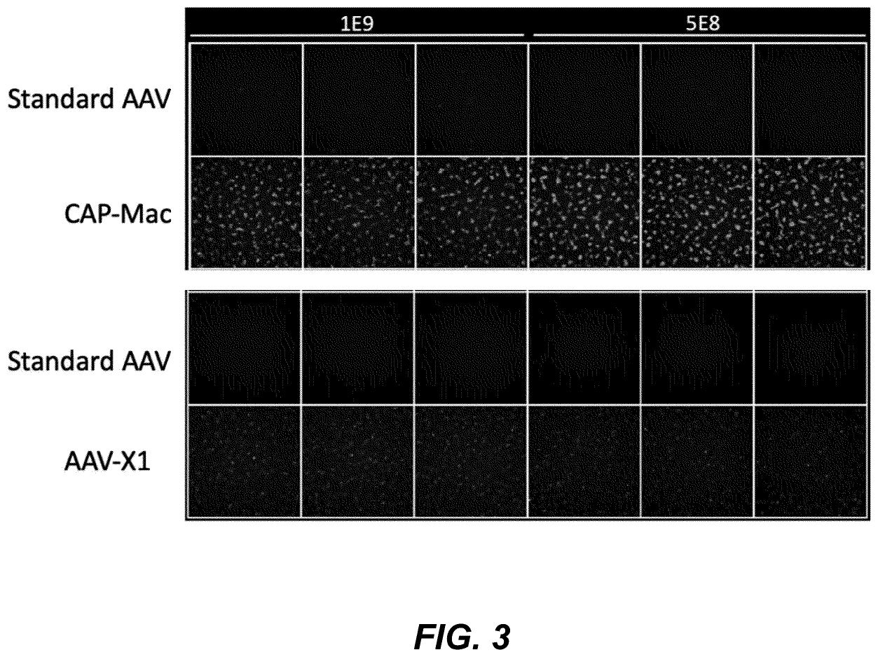

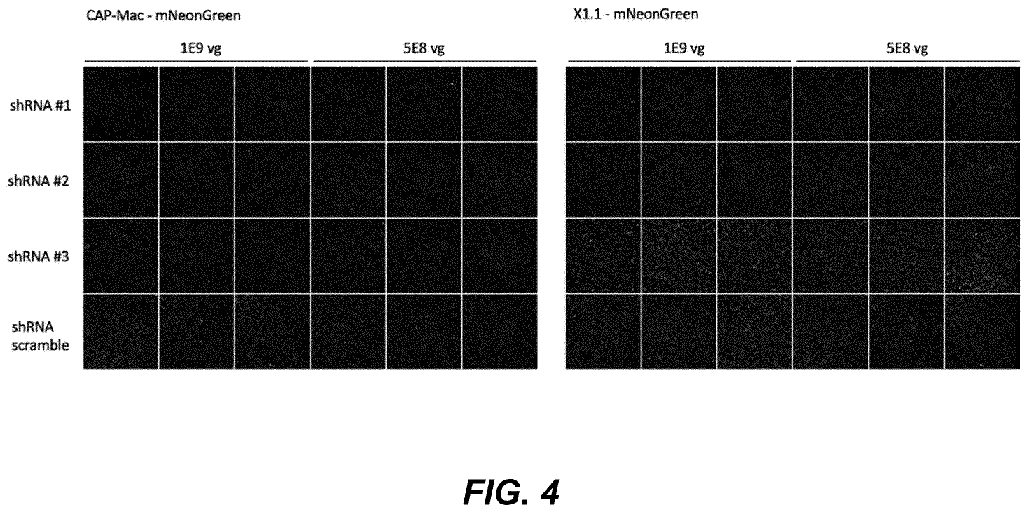

LRP6 is strongly expressed in the human BBB and is a target for AAV-X1.1's enhanced potency in the CNS across species. AAV-X1.1 and CAP-Mac were found to strongly and directly bind human LRP6 extracellular domain by SPR, with CAP-Mac binding appearing more potent, while no such interaction was found for AAV9 (FIG. 1). Computational modeling of AAV-X1.1 insertion peptide with human LRP6 extracellular domain shows binding to domain 2 while CAP-Mac's insertion peptide displays binding with either domain 1 or domain 2 (FIG. 2). Both of these engineered AAVs display enhanced potency on human HEK293 cells (which endogenously express LRP6) compared to standard AAV9-based AAVs (FIG. 3). Knockdown of endogenous LRP6 in HEK293 cells selectively reduces the potency of both AAV-X1.1 and CAP-Mac (FIG. 4).

These targets allow target-based engineering of AAVs to modulate biodistribution, potency, and host immune interaction. They also allow target-based engineering for therapeutic antibodies, peptides, Fabs, scFv, nanobody, and alternative protein scaffolds as well as ASO, and small molecules to gain properties of natural and engineered AAVs.

TABLE 1
TEST AND CONTROL SAMPLES

| | | | Molecular | Screening |
|---|---|---|---|---|
| Sample ID | Sample Type | Target(s)/Receptor(s) | Weight | Concentration |
| CAP-C1 | Test AAV | KIAA0319L (Isoform 1) | 3.8 MDa | 3 × 10⁴ particles/cell |
| CAP-B22 | Test AAV | Mouse SCA1(LY6A) and | 3.8 MDa | 3 × 10⁴ particles/cell |
| | | KIAA0319L (Isoform 1) | | |
| X1B10 | Test AAV | KIAA0319L (Isoform 1) | 3.8 MDa | 3 × 10⁴ particles/cell |
| PNS1 | Test AAV | KIAA0319L (Isoform 1) | 3.8 MDa | 3 × 10⁴ particles/cell |
| PNS2 | Test AAV | KIAA0319L (Isoform 1) | 3.8 MDa | 3 × 10⁴ particles/cell |
| AAV9 | Control AAV | KIAA0319L (Isoform 1) | 3.8 MDa | 3 × 10⁴ particles/cell |

### Methodology

Library Screen

A test AAV pool containing 3×10⁴ particles/cell of CAP-C1, CAP-B22, PNS1, PNS2 and AAV9 (Control AAV) and 1×10⁴ particles/cell of X1B10 (was screened for binding against fixed HEK293 cells/slides expressing duplicate 6019 human plasma membrane proteins, secreted and cell surface tethered human secreted proteins as well as 397 human heterodimers (18 slide sets, n=2 slides per slide set) using the direct fix method (following sample addition, AAVs were not removed and slides were fixed). All transfection efficiencies exceeded the minimum threshold. An anti-AAV9 antibody (Anti-Adeno-associated Virus 9, clone HL2372, supplied by Merck, Cat #MABF2309-100UL, 1:500 dilution) followed by AlexaFluor647 anti-mIgG H+L detection antibody was used. In total, 22 library hits (duplicate spots) were identified by analyzing fluorescence (AF647 and ZsGreen1) on ImageQuant. There were a range of intensities (signal to background) from very weak to medium/strong.

Confirmation/Specificity Screens

Vectors encoding all 22 hits, plus vectors encoding Mouse SCA1 (LY6A) and KIAA0319L and control vectors encoding CD86 and EGFR, were spotted in duplicate on new slides, and used to reverse transfect human HEK293 cells. All transfection efficiencies exceeded the minimum threshold. Transfected HEK293 cells were treated after fixation with 3×10⁴ particles/cell of CAP-C1, CAP-B22, PNS1, PNS2 or AAV9 (Control AAV), 1×10⁴ particles/cell of X1B10, 0.2 μg/mL of CTLA4-mFc (positive control), or no test molecule (secondary only; negative control) using the direct fix method as described above (n=2 slides per treatment, NB. controls were also fixed after sample incubation). Slides were analyzed as described above. Hits were categorized as specific, or non-specific (e.g., also came up with either the positive control or a negative control).

TABLE 2
SPOTTING PATTERN

| Position | Gene ID |
|---|---|
| 1 | Mouse SCA1 (LY6A) |
| 2 | LDLR |
| 3 | FCGR1A |
| 4 | GP2 |
| 5 | KIAA0319L (undefined) |
| 6 | ADA |
| 7 | DPP4 |
| 8 | LRP6 |
| 9 | FAM234A |
| 10 | ANPEP |
| 11 | KIAA0319L (Isoform 1) |
| 12 | GGT1 |
| 13 | PLB1 |
| 14 | SPNS2 |
| 15 | CSF2 |
| 16 | CXCL12 |
| 17 | IL3 |
| 18 | IGF2 |
| 29 | DKK3 |
| 20 | EPYC |
| 21 | GP2 |
| 22 | IGF1 |
| 23 | ALB |
| 24 | MFSD2B |
| 25 | CD86 |
| 26 | EGFR |

In sum, test AAVs CAP-C1, CAP-B22, PNS1, PNS2, AAV9 (Control AAV) and X1B10 were screened for binding against human HEK293 cells expressing 6019 human plasma membrane proteins, secreted and cell surface-tethered secreted proteins plus 397 human heterodimers. CAP-C1 showed significant specific interactions with GP2, DPP4, IL3 and DKK3. CAP-B22 showed significant specific interactions with one of its primary targets, Mouse SCA1 (LY6A) and with DPP4, FAM234A, IL3 and DKK3. X1B10 showed specific interactions with GP2, DPP4, LRP6, ANPEP, CSF2, IL3, DKK3 and EPYC. PNS1, PNS2 and AAV9 (Control AAV) all showed significant specific interactions with DPP4, IL3 and DKK3.

### Example 2

### Human Cell Surface-AAV Interactomes Identify LRP6 as Blood-Brain-Barrier Transcytosis Receptor and Immune Cytokine IL3 as AAV9 Binder

Adeno-associated viruses (AAVs) are foundational gene delivery tools for basic science and clinical therapeutics. However, lack of mechanistic insight, especially for engineered vectors created by directed evolution, can hamper their application. As described herein, an unbiased human cell microarray platform was adapted to determine the extracellular and cell surface interactomes of natural and engineered AAVs. Identified was a naturally-evolved and serotype-specific interaction of AAV9 with human interleukin 3 (IL3), which, without being bound by any particular theory, may play roles in host immune response. Also identified was lab-evolved low-density-lipoprotein-receptor-related-protein 6 (LRP6) interactions specific to engineered capsids that cross the blood-brain barrier in non-human primates upon intravenous administration. The unbiased cell microarray screening approach also allowed identification of off-target tissue binding interactions of engineered brain-enriched AAVs that inform vectors' peripheral organ tropism and side effects. These results allow confident application of engineered AAVs in diverse organisms and unlock target-informed engineering of improved viral and non-viral vectors for non-invasive therapeutic delivery to the brain.

Adeno-associated viruses (AAVs) have become the gene delivery vector of choice at the bench and in the clinic. Systemic administration of AAVs allows noninvasive targeting, particularly of large or distributed biological structures, but access to the brain from the periphery is restricted by the blood-brain barrier (BBB), a complex biological structure that regulates molecular access to the central nervous system (CNS). Systemic administration of AAVs also exposes the vectors to the host immune system and off-target tissues. The poor efficiency of brain targeting after systemic administration with natural serotypes often necessitates high doses that raise costs and may trigger adverse side effects. Thus, improved vectors are needed if AAV gene therapy is to realize its full therapeutic potential.

AAV capsid engineering, particularly through directed evolution methods, has demonstrated that markedly improved potency in desired cell types and tissues after systemic intravenous delivery is possible. As AAV capsids are applied across species however, the enhanced tropisms of many engineered vectors can vary. This is concerning for human clinical trials where a capsid developed in non-human species that performs poorly when translated may not only fail to provide therapeutic benefit but might preclude that patient from future therapies by inducing neutralizing antibodies.

This translational challenge of AAV engineering through directed evolution also represents an opportunity to better understand fundamental mechanisms of drug delivery to the brain. Directed evolution of engineered capsids with enhanced BBB crossing provides a platform with which researchers may survey the most efficient pathways through this barrier. While recent progress suggests that engineered AAVs may utilize diverse BBB-crossing receptors, the mechanisms of primate brain-enhanced vectors remain underexplored.

To address this challenge, as described herein, Retrogenix cell microarrays of the human membrane proteome and secretome were adapted to screen natural and engineered AAV capsid interactions with host cells. This allowed for rapid assay of more than 90% of the human membrane proteome and secretome, including key protein classes such as receptors, transporters, and cytokines. Using this broad, unbiased screen, several new AAV interactions were identified with implications for the host immune response (human interleukin 3 (IL3) binding to AAV9), enhanced BBB crossing across species (via low-density-lipoprotein-receptor-related-protein 6 (LRP6) binding by AAV9-X1.1 and CAP-Mac), and off-target tissue tropism (through glycoprotein 2 (GP2) binding by AAV9-X1.1 and CAP-Mac). Understanding the mechanism of action of systemic AAVs through methods such as those used here will be critical for successful vector translation and enables design of improved vectors, as well as other therapeutic protein modalities, for specific targets.

### High-Throughput Screening for AAV Binding Partners

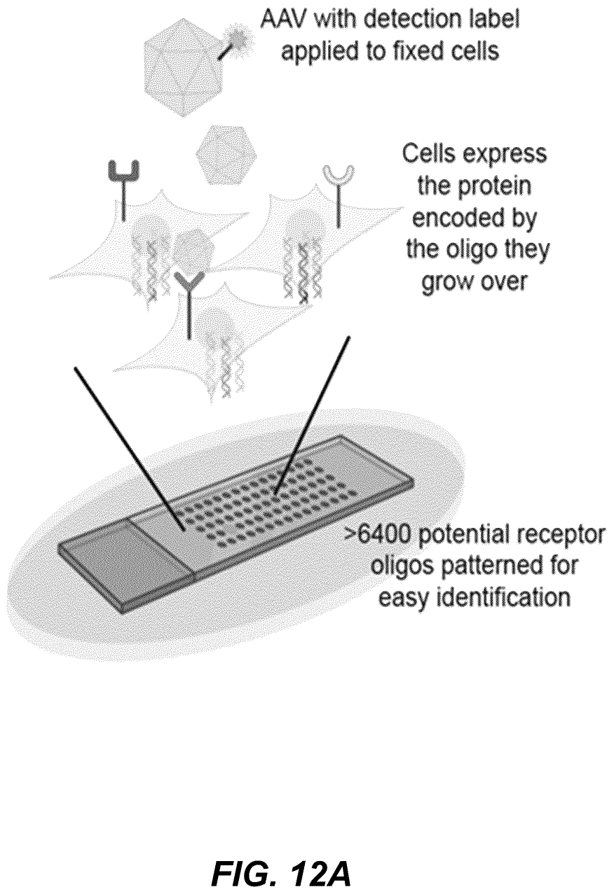

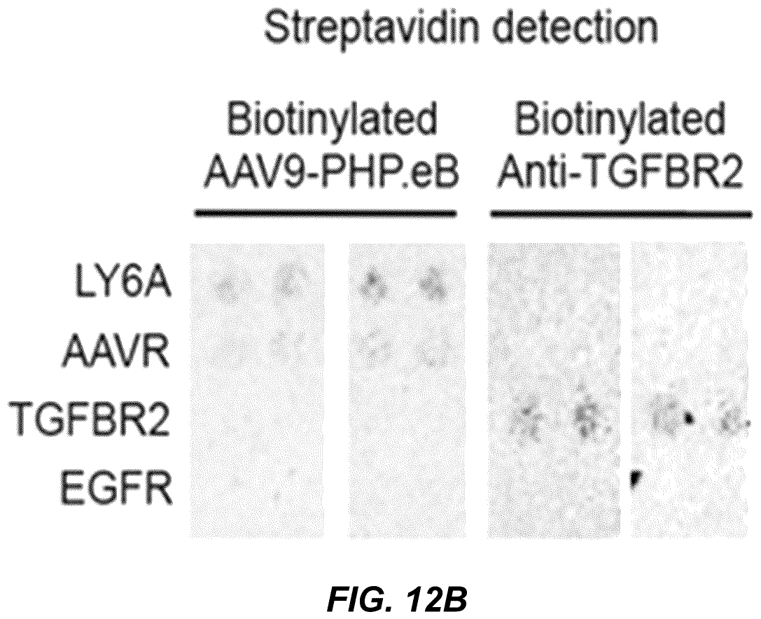

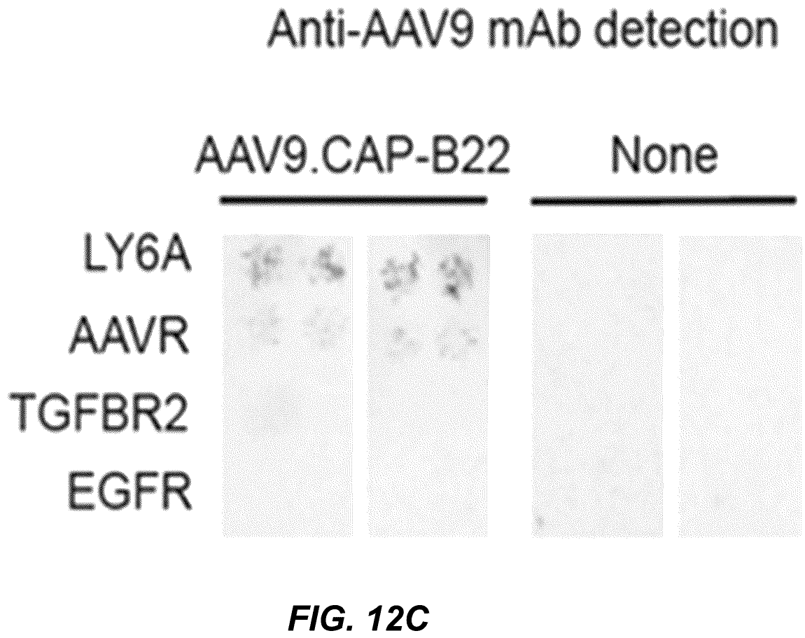

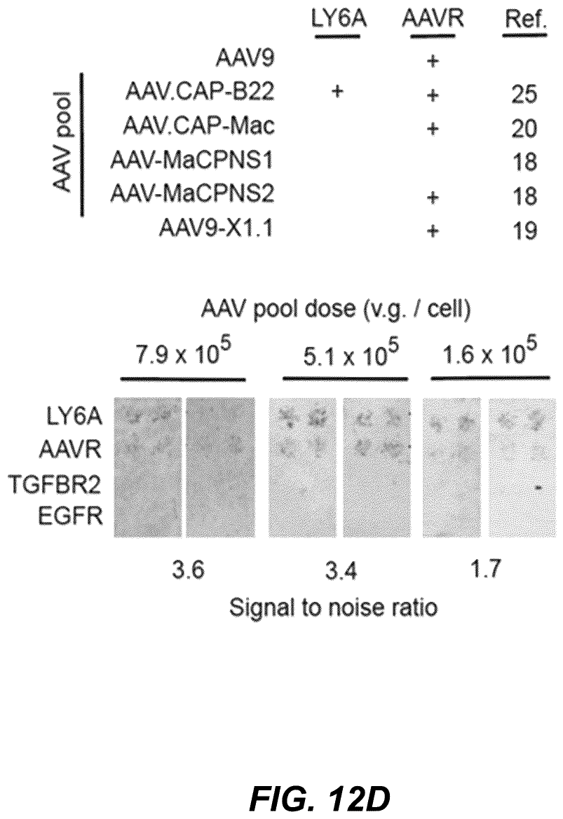

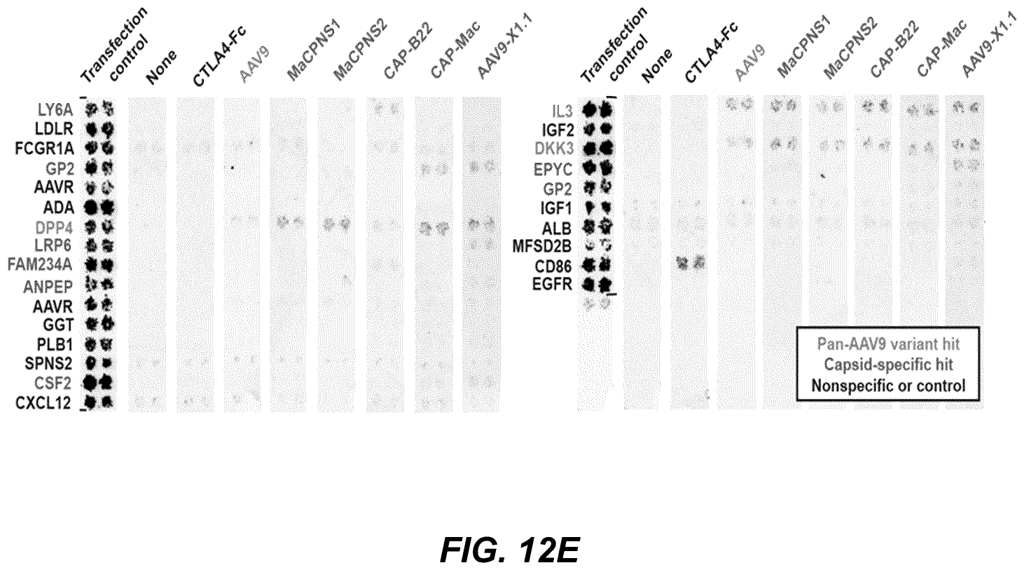

To screen AAV-binding proteins, Retrogenix cell microarrays of the human membrane proteome and secretome were used, in which DNA oligos encoding human membrane and secreted proteins are affixed at known slide locations (FIG. 12A). HEK293 cells are then grown on the slides and become individually reverse-transfected with the oligos in the corresponding pattern. AAVs that directly interact with a given protein will preferentially bind to cells expressing that protein; other slide locations define non-specific background binding. To increase confidence in binding specificity, each protein is patterned at two different locations (four locations presented for initial condition optimization) (FIG. 12B-FIG. 12C). Screen conditions were optimized using previously-identified AAV and interacting protein pairs, (1) AAV9 with AAVR (KIAA0319L) and (2) PHP.eB with mouse LY6A, for two different detection methods: biotin tagging and direct detection (FIG. 12B-FIG. 12C). Biotinylated capsids can be detected with fluorescent streptavidin, and unlabeled capsids are detected with an antibody whose epitope is distinct from the commonly engineered capsid variable regions IV and VIII. In some embodiments, capsid primary amine labeling levels must be tuned so that surface modification does not interfere with capsid key binding interactions. It was found that the best signal to noise ratio for duplicate spots (calculated as the average intensity across positive control spots compared to the average intensity of the rest of the slide) was achieved by directly fixing cell-bound AAVs without washes.

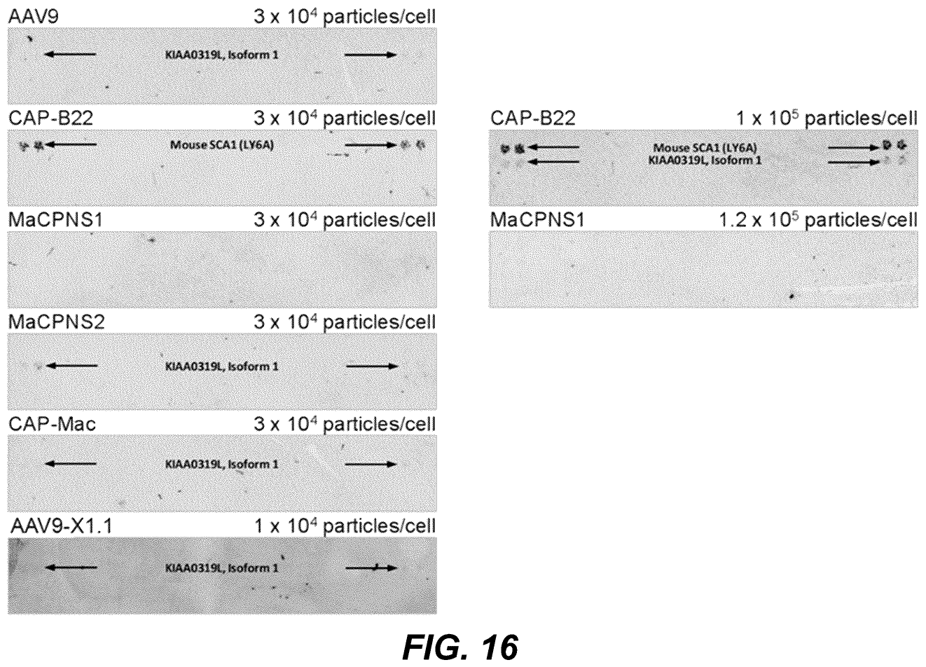

Next, direct capsid detection and validated conditions with a panel of AAV capsids, including AAV9 as well as five engineered AAV9 variants with enhanced potency in the CNS of non-human primates (NHPs) after systemic administration, was investigated (Table 3 and FIG. 12D). Testing these capsids individually revealed that all AAVs except MaCPNS1 exhibited detectable AAVR binding (the exception may be due to the geometry of the capsid's variable region VIII insertion), whereas only CAP-B22 interacted with mouse LY6A (likely through the PHP.eB loop in variable region VIII) (FIG. 12D and FIG. 16). To enable higher-throughput screening, the six capsids were tested as a pool. Pooled testing required additional dosage optimizing, first for the individual and then for the collective background binding levels of the included capsids (Table 5). An optimal dose was determined that minimized background binding while still allowing the unique interaction of CAP-B22 with mouse LY6A to be distinguished from the five non-LY6A-interacting capsids (FIG. 12D).

After these controls, the six-capsid pool was tested in a full screen of approximately 6400 proteins, including 6000 human plasma membrane proteins and secreted and cell surface-tethered proteins, as well as approximately 400 heterodimers. 22 library hits were identified in which each duplicate spot showed enhanced signal over background. To assign these hits to specific capsids, follow-up deconvolution screens were performed with each individual capsid from the pool (FIG. 12E). DNA oligos for the 22 identified hits, as well as the positive control CD86, were affixed in duplicate locations to new slides. A negative control condition with no AAV analyte and a positive control condition with CTLA4-Fc (CD86 binder) were also included. Hits were successfully assigned, including both membrane-localized and secreted proteins, to capsids. Some of these interactions were unique to specific AAV9 variants, while others were conserved across all capsids tested (Table 4).

### Validation of Individual AAV Binding Interactions

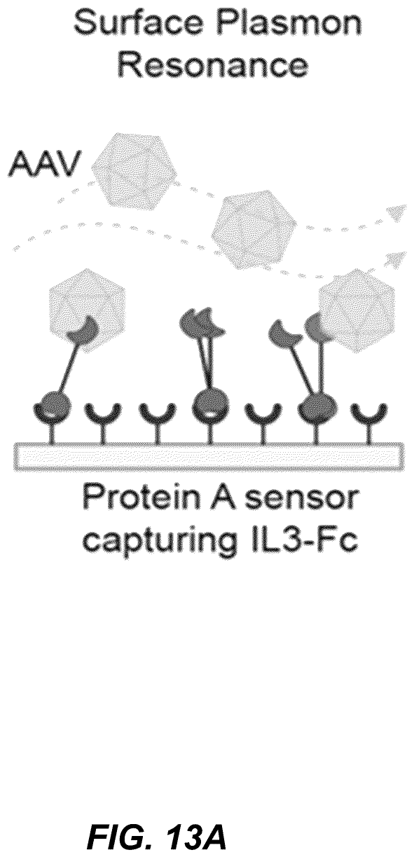

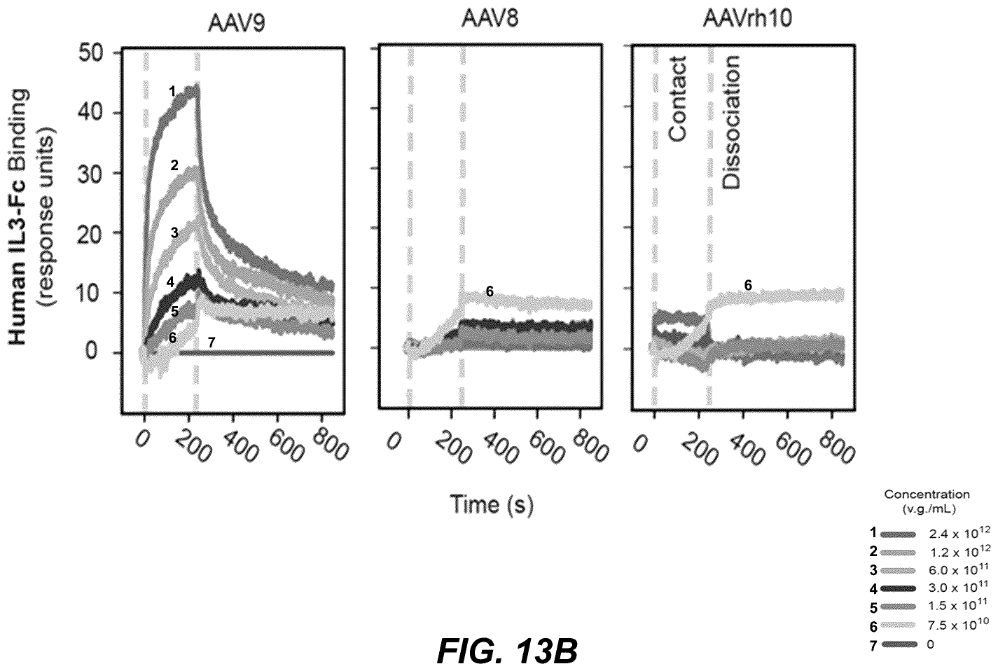

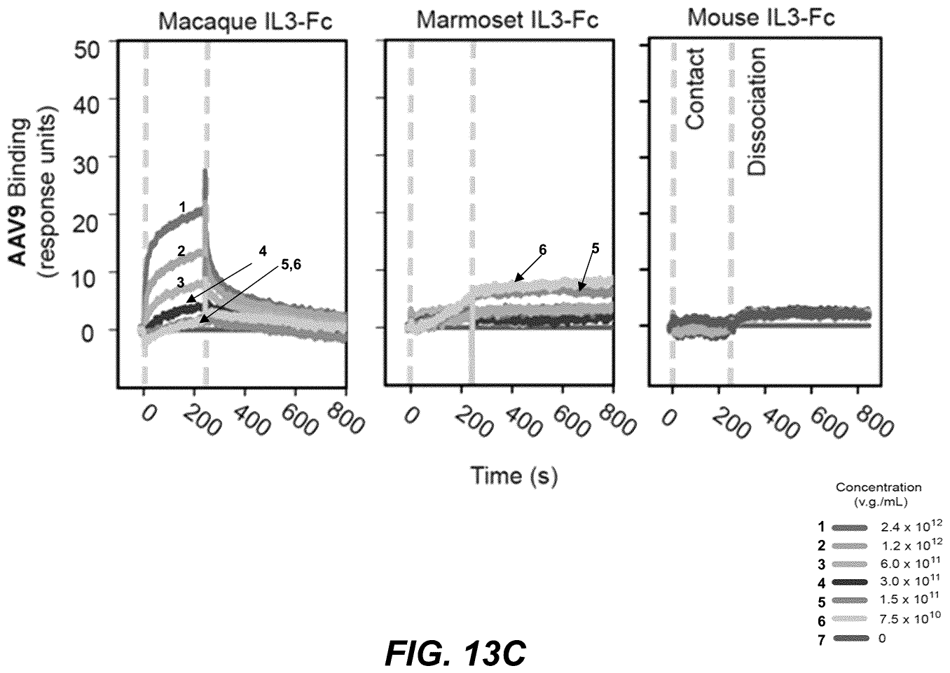

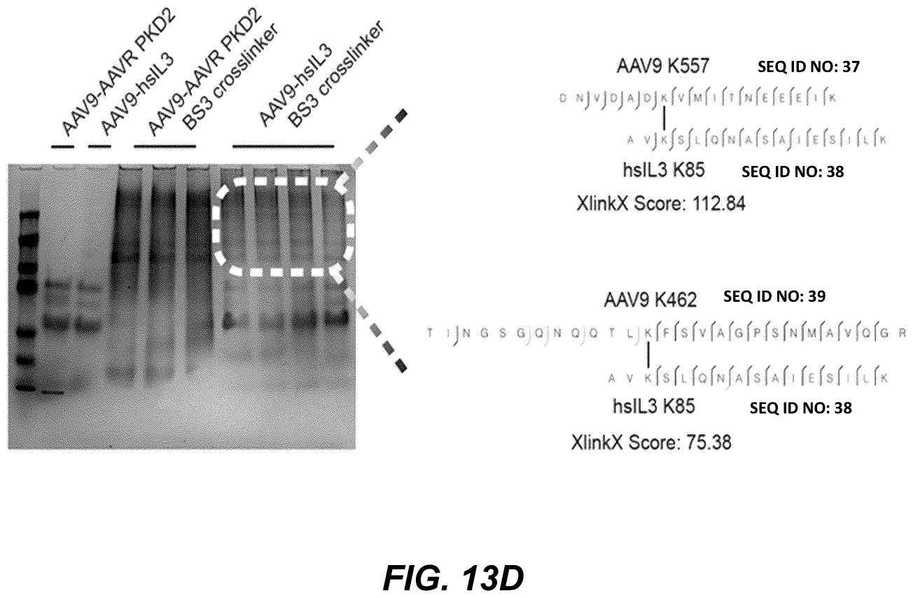

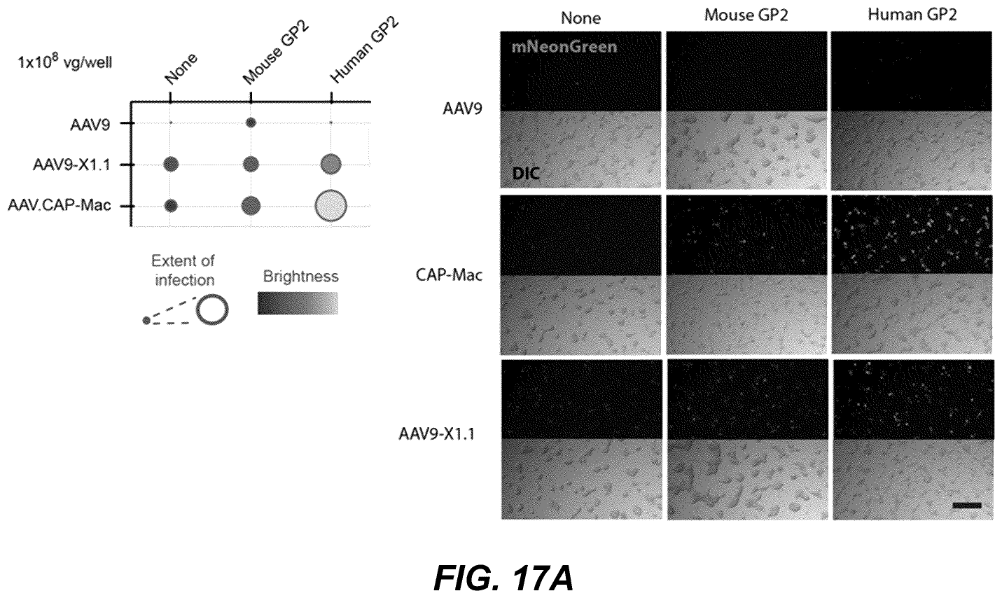

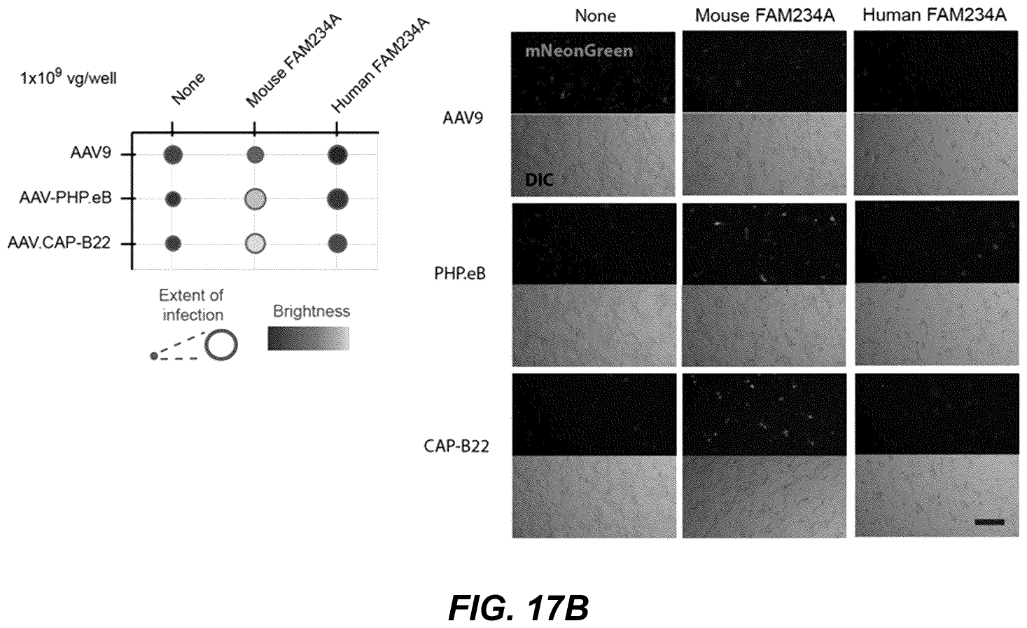

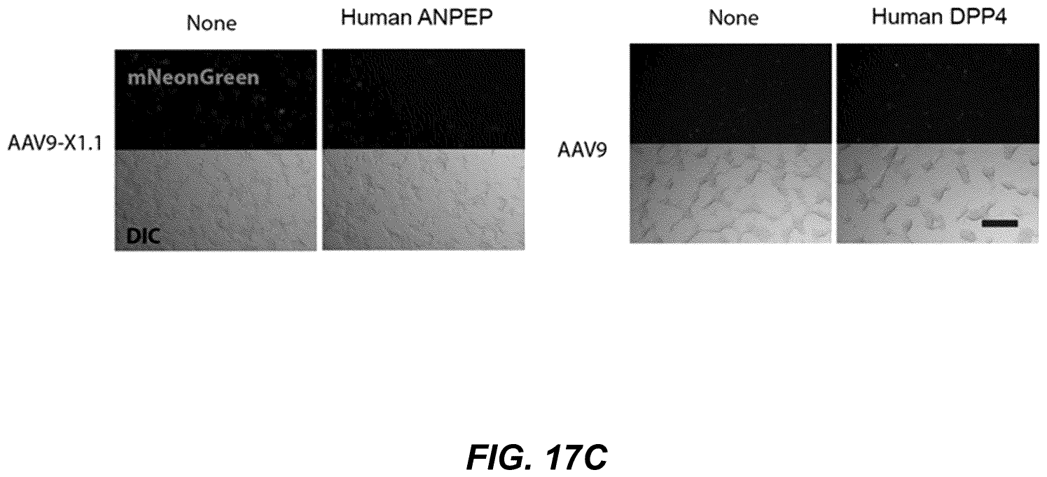

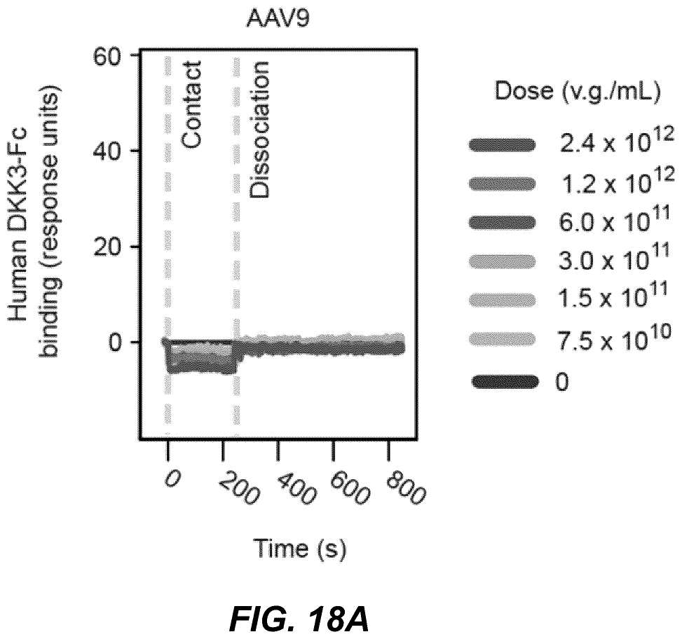

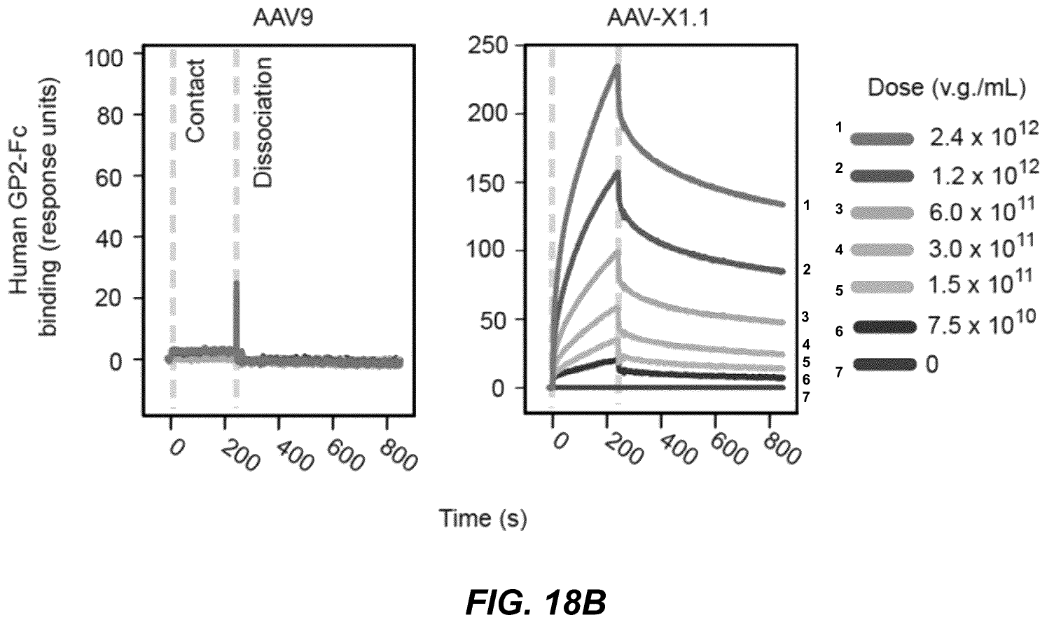

To validate binders from the cell microarray screen, hits were tested for their ability to enhance AAV potency in cell culture (FIG. 17A-FIG. 17C) and capsid-binding interactions were characterized by surface plasmon resonance (SPR) (FIG. 13A and FIG. 18A-FIG. 18B). This reduced the candidate receptors to a subset of validated interactors (Table 4). In analyzing these interactions, the identified interaction of AAV9 and all its lab-evolved derivatives with the human immunomodulatory protein interleukin-3 (IL3) was striking, because AAVs are relatively well tolerated by the immune system. IL3 is produced by activated T cells as part of the inflammatory response to viral infection, triggering expansion of various immune cells and activating type I interferon-secreting plasmacytoid dendritic cells. Using SPR, it was found that AAV9 binds human IL3 but not the closely related natural serotypes AAV8 and AAVrh10 (FIG. 13B). IL3 from different species were tested next, and it was found that AAV9 binds to human and macaque IL3 (83% sequence identity) but not marmoset or mouse IL3 (69% and 27% sequence identity, respectively)(FIG. 13B), suggesting a divergence between new and old world monkeys.

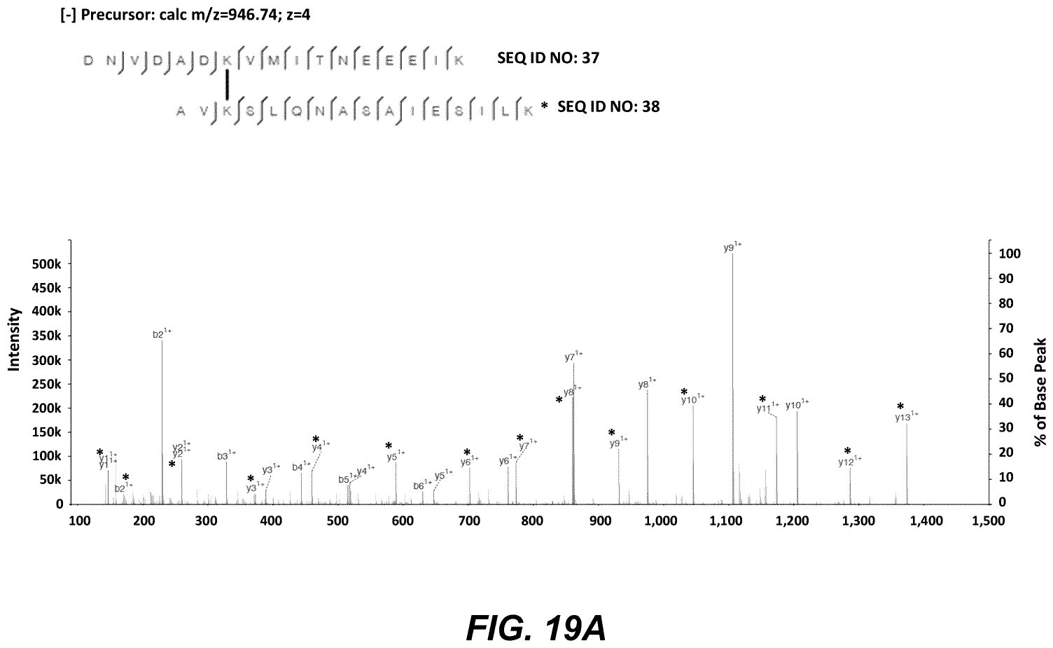

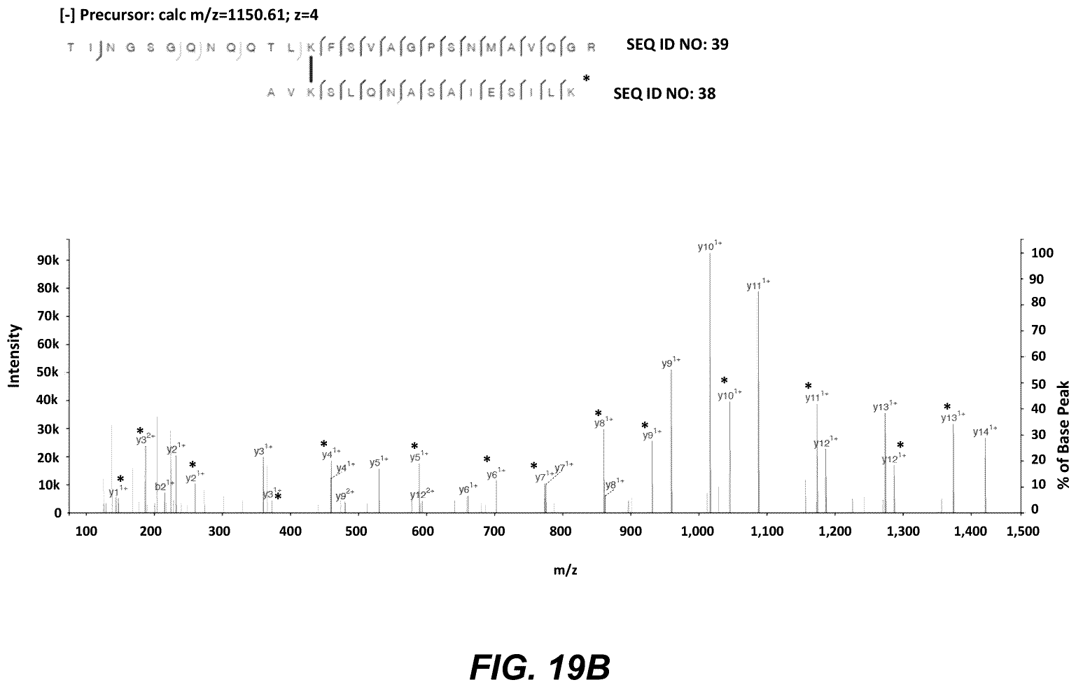

To understand the species and serotype specificity of IL3's interaction with AAV9, the structure of the bound complex was investigated. As functional AAV ligands may have weak and dynamic monomeric interactions, avidity was leveraged by flowing the 60-mer AAV9 capsid over protein A-captured dimeric IL3-Fc to detect all biologically meaningful interactions. Despite this high avidity in the SPR experiment, the apparent affinity of the interaction was consistent with only a high nM interaction. Therefore, chemical crosslinking of IL3-bound AAV9 was performed, followed by mass spectrometry (XL-MS). Using bis(sulfosuccinimidyl)suberate (BS3) crosslinking agent, 2 crosslinks between the proteins were detected (FIG. 13D and FIG. 19A-FIG. 19B). These cross-links place IL3 at the base of the 3-fold symmetry spike and in the 2-fold symmetry valley but do not allow for the generation of a binding model.

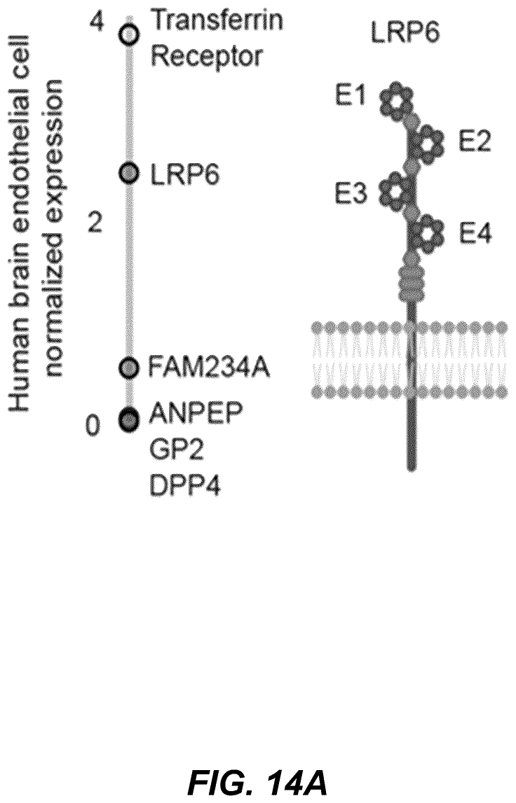

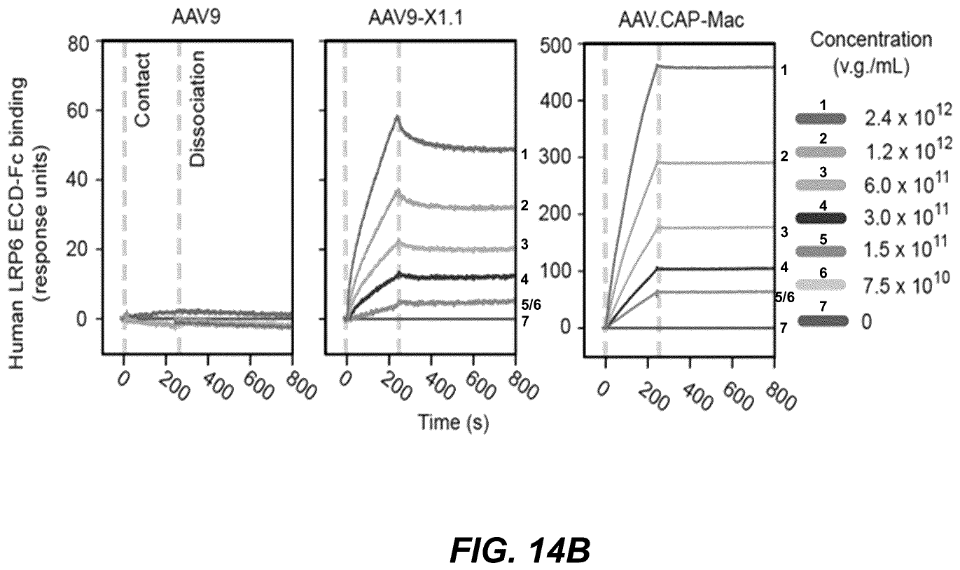

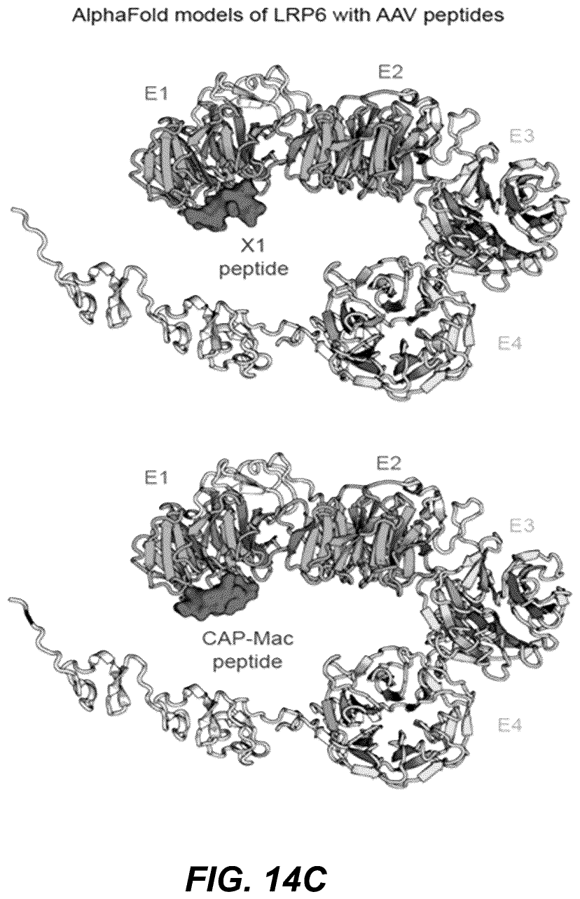

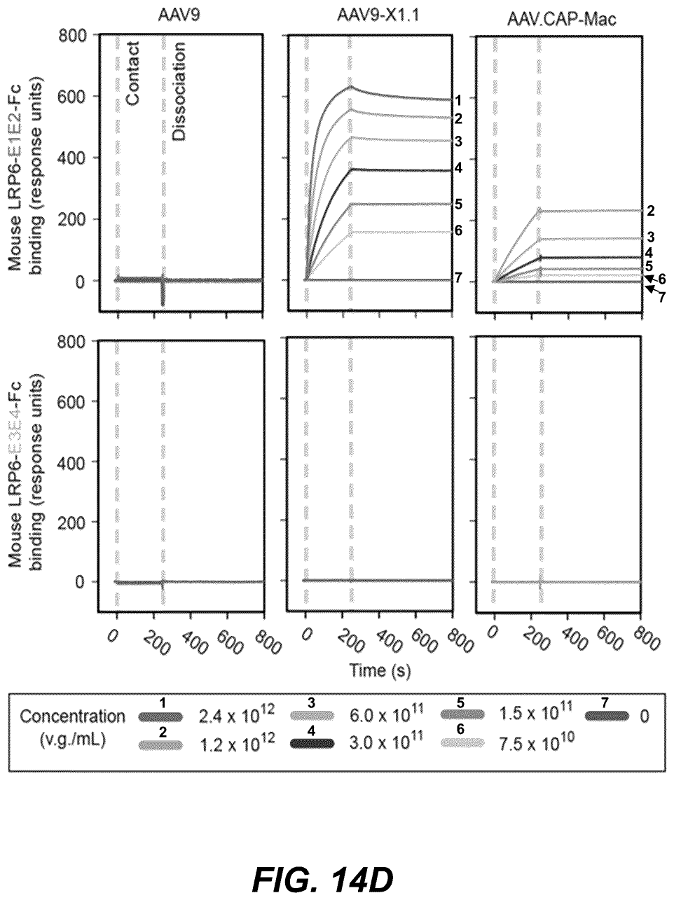

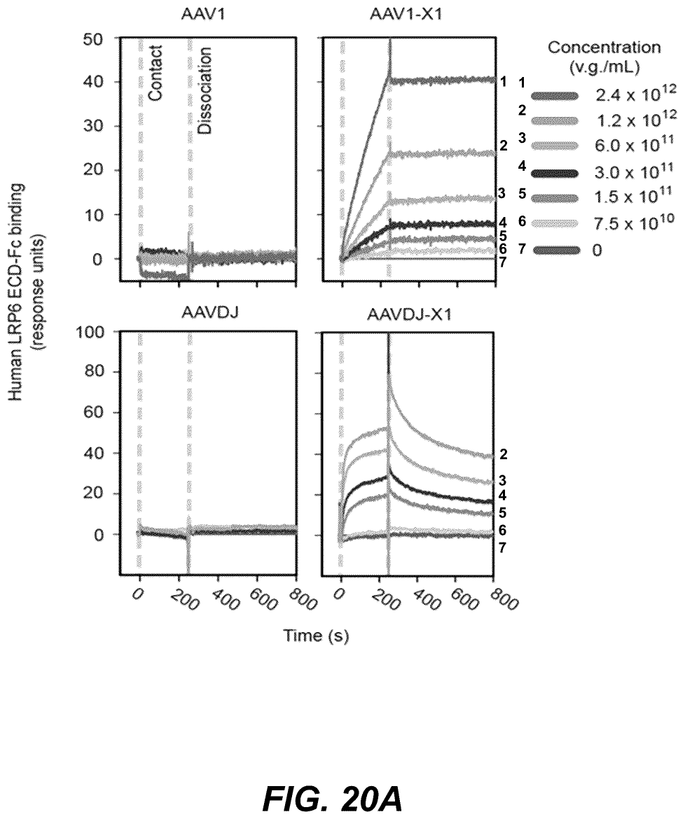

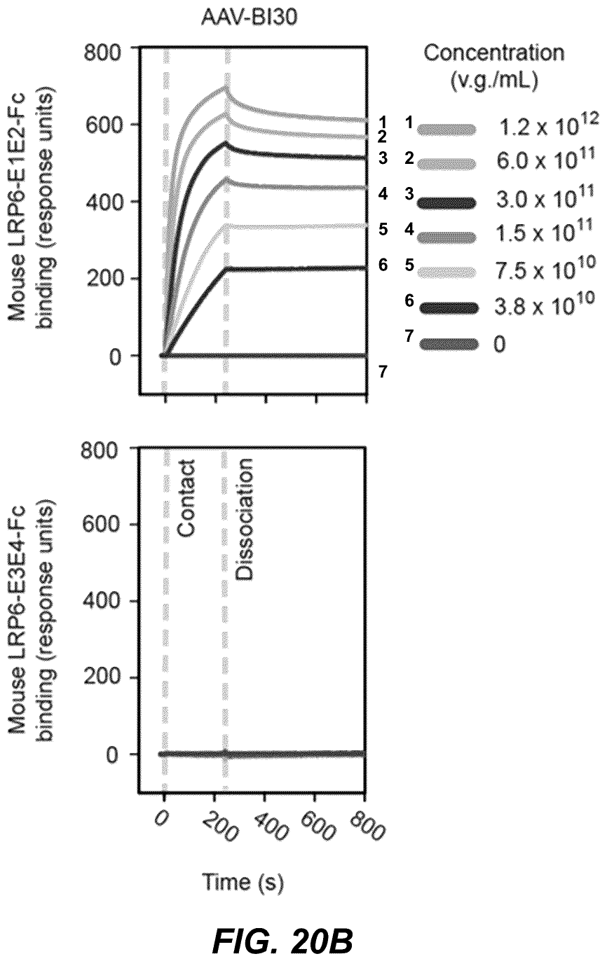

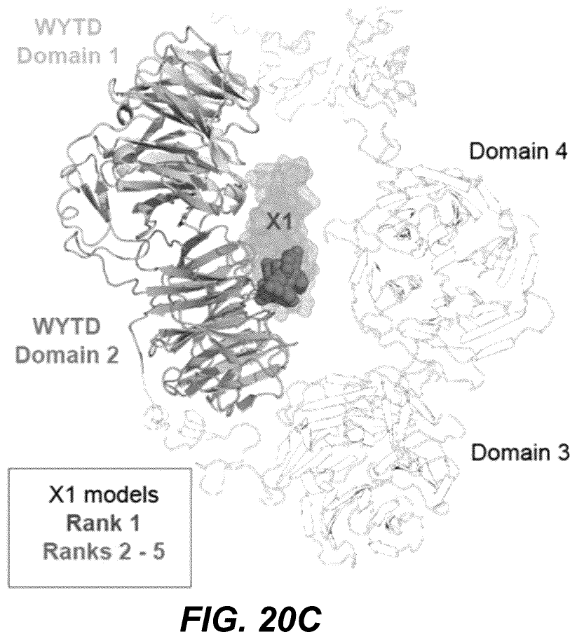

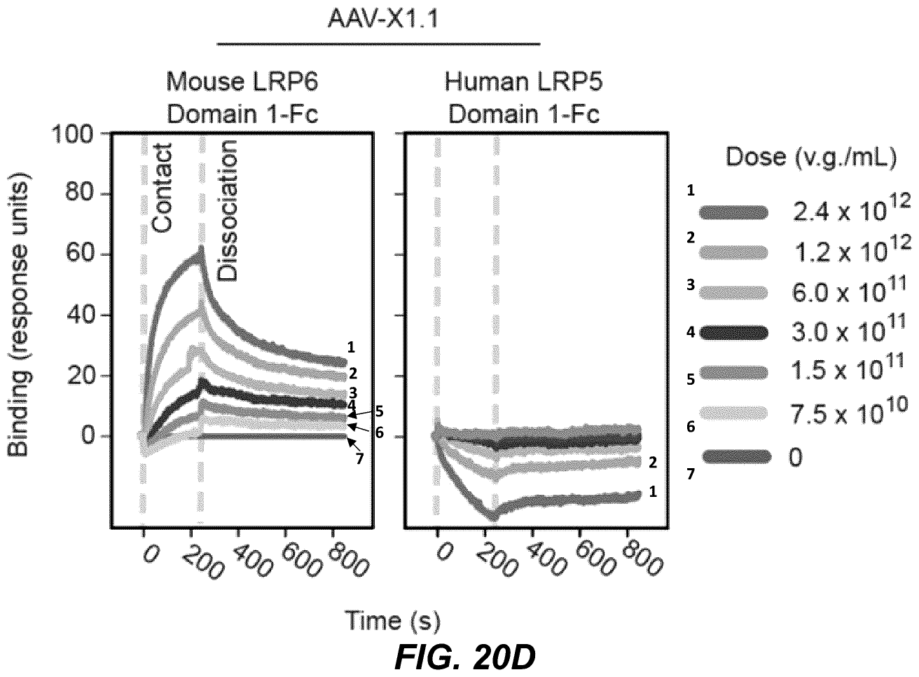

The validated AAV interactors were then assessed for their potential to explain the enhanced brain tropisms of the engineered capsids. Sorting the screen hits by their expression level in endothelial cells of the human BBB spotlighted a specific interaction of low-density-lipoprotein-receptor-related-protein 6 (LRP6) with AAV9-X1.1 (FIG. 14A). This capsid displays an enhanced brain endothelial-specific tropism in mice that shifts to an enhanced neuronal tropism in macaque (Table 3). Although AAV9-X1.1 contains modifications from AAV9 at both variable regions IV and VIII (Table 3), the tropism of AAV9-X1.1 can be transferred to other natural serotypes such as AAV1 and AAV-DJ by transferring only the variable region VIII insertion of AAV9-X1.1. By SPR, it was confirmed that the X1 peptide insertion in variable region VIII endows AAV1-X1 and AAVDJ-X1, but not their unmodified parent serotypes, with LRP6 binding (FIG. 20A). This demonstrates that the functional modularity of the X1 peptide in different AAV serotypes in vivo corresponds to LRP6-binding modularity.

LRP6 is a coreceptor of the canonical Wnt signaling pathway, with developmental and homeostatic roles in many tissues. The high degree of LRP6 sequence conservation across species (98% and 99.5% sequence identity conserved between human LRP6 and mouse or macaque LRP6, respectively) aligns with AAV9-X1.1's enhanced tropisms compared to AAV9 in rodents and primates. A similar enhancement in tropism across species is also seen for CAP-Mac (Table 3), which was engineered in marmosets and has enhanced endothelial tropism compared to AAV9 in marmosets as well as enhanced neuronal tropism in macaque. Therefore, CAP-Mac was also tested by SPR for interaction with the human LRP6 extracellular domain (FIG. 14B). As with IL3, avidity was utilized to ensure that weak yet functionally important interactions are captured. Both AAV9-X1.1 and CAP-Mac strongly bind human LRP6-Fc, unlike their parent capsid, AAV9, with a sub-nM apparent affinity.

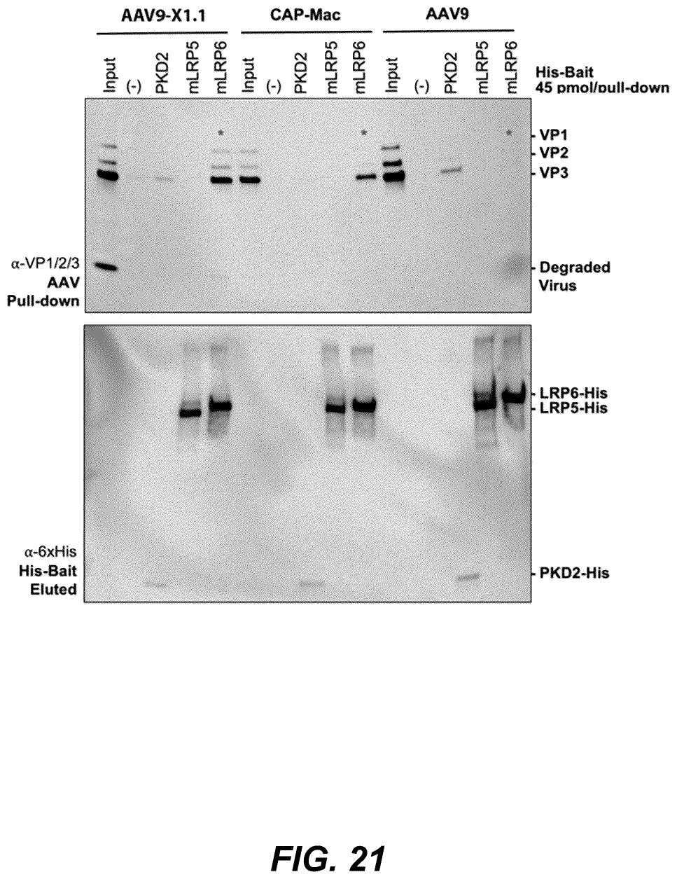

LRP6 has many endogenous WNT signaling partners with binding sites spanning either extracellular YWTD domains 1 and 2 (E1E2) or domains 3 and 4 (E3E4). AlphaFold-Multimer was applied to build models of the AAV interaction complexes, which predicted that the X1 and CAP-Mac variable region VIII peptides bind LRP6 YWTD domain 1 (FIG. 14C). While the cooperative folding of E1 and E2 complicates testing of individual domains, SPR results from mouse LRP6 extracellular domain fragments were consistent with the model prediction, with interaction observed for LRP6-E1E2 but not LRP6-E3E4 (FIG. 14D). AAV-BI30 was also tested, which is another engineered capsid with specific expression in the mouse brain endothelium (Table 3), finding that it also binds to LRP6-E1E2 but not LRP6-E3E4 (FIG. 20B). A pull-down assay confirmed that both AAV9-X1.1 and CAP-Mac bind the full length extracellular domain of mouse LRP6 but not that of the closely-related LRP5 (FIG. 21). AAV9, on the other hand, bound only to AAVR's PKD2, as reported previously.

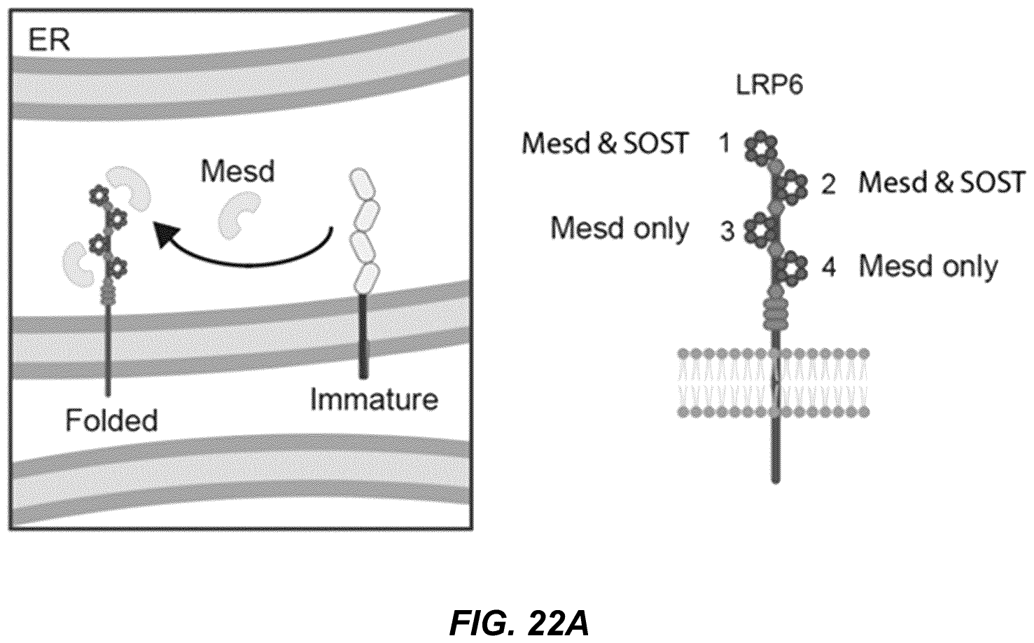

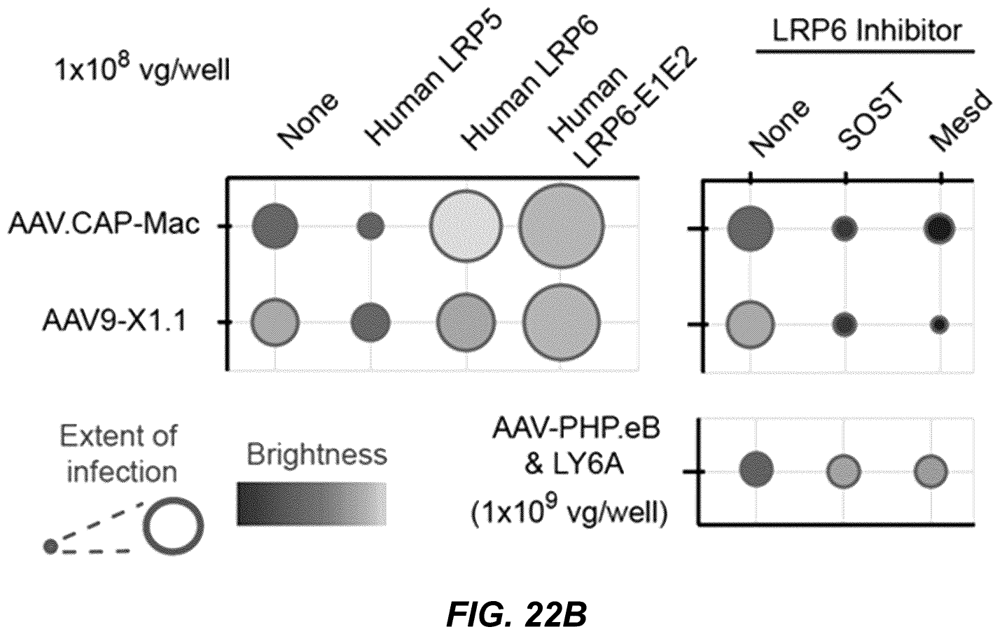

AAV9-X1.1 and CAP-Mac potently infect HEK293 cells, with AAV9-X1.1 having a stronger effect (FIG. 22B). To determine if this potency is mediated by endogenous LRP6 expression in HEK293 cells, the vectors were tested with LRP6 inhibitors. AAV9-X1.1 potency was markedly reduced by Mesoderm development LRP chaperone (Mesd), a natural endoplasmic reticulum chaperone and recombinant extracellular inhibitor of LRP5 and LRP6, and Sclerostin (SOST), which inhibits LRP6 through binding only E1E2 (FIG. 22A-FIG. 22B). Importantly, neither Mesd nor SOST inhibited the potency of PHP.eB on LY6A overexpressing cells. Transient overexpression of human LRP6 boosted the potency of both capsids, with a stronger effect for CAP-Mac (FIG. 22B). This effect was largely preserved when a truncated LRP6-E1E2 was used. As expected from our pull-down assay, transient overexpression of LRP5 did not enhance the potency of either CAP-Mac or AAV9-X1.1. Together, these results support a specific functional interaction between LRP6 and both CAP-Mac and AAV9-X1.1, although the two capsids may have different functional sensitivities to LRP6 expression level. While AAV9-X1.1 more productively engages LRP6 at the lower endogenous expression levels of LRP6, CAP-Mac shows more enhanced potency after transient overexpression of LRP6.

Of note, in addition to the intended CNS tropism receptors gained through these AAV capsid engineering efforts, both LRP6-binding engineered capsids also gained interactions with the GPI-linked protein glycoprotein 2 (GP2), which has specific pancreatic expression and, in a secreted form, plays antibacterial roles in the gut (FIG. 17A, FIG. 18A-FIG. 18B, and Table 4). GP2 boosted the potency of both capsids in cell culture, with a stronger effect for the human protein than the mouse (FIG. 17A).

FAM234A, which bound CAP-B22 in the cell microarray screen, is found in the brain with low expression across many neuron types. Although, FAM234A has been identified in disease-association studies, no specific molecular function has been assigned to this protein. FAM234A enhances the potency of both CAP-B22 and PHP.eB in cell culture, with the mouse receptor showing a stronger effect than the human protein (FIG. 17B). This suggests that the interaction is driven by the capsids' shared variable region VIII insertion sequences (Table 3).

### Engineered AAVs Utilize LRP6 at the Blood-Brain Barrier in Mouse and Primate

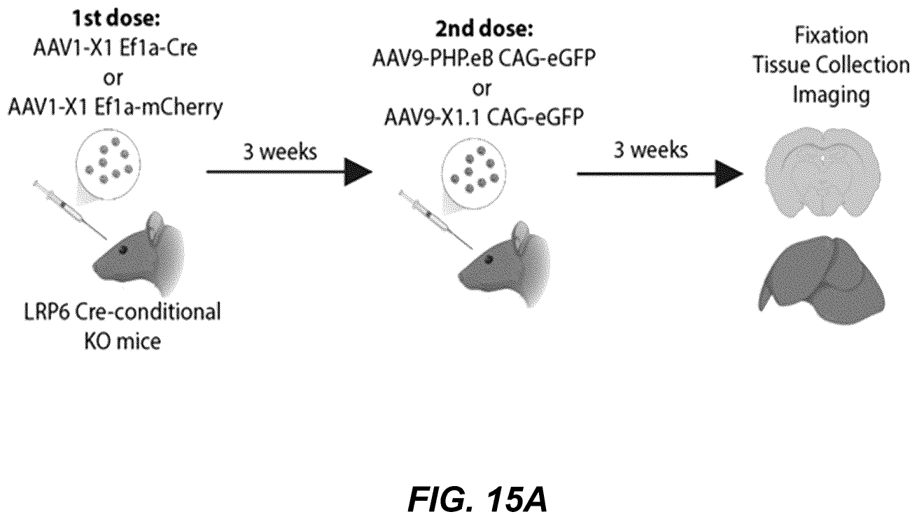

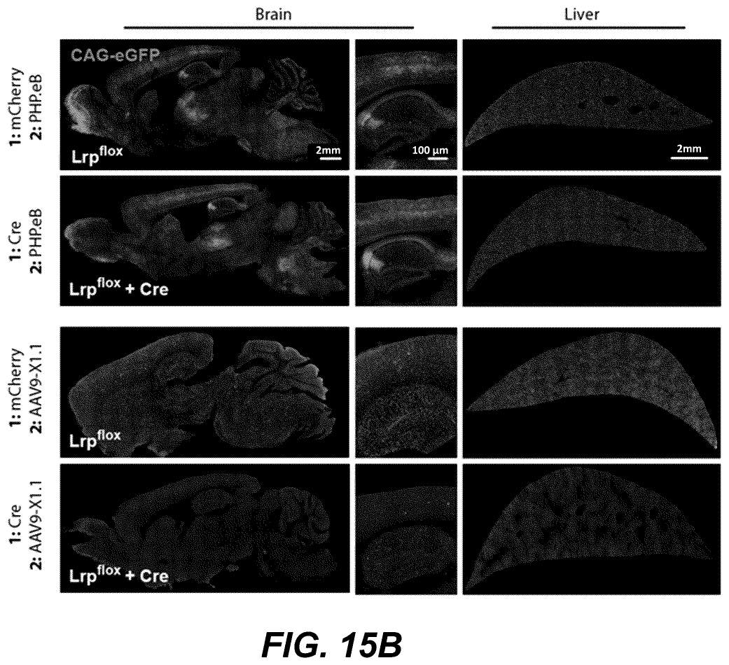

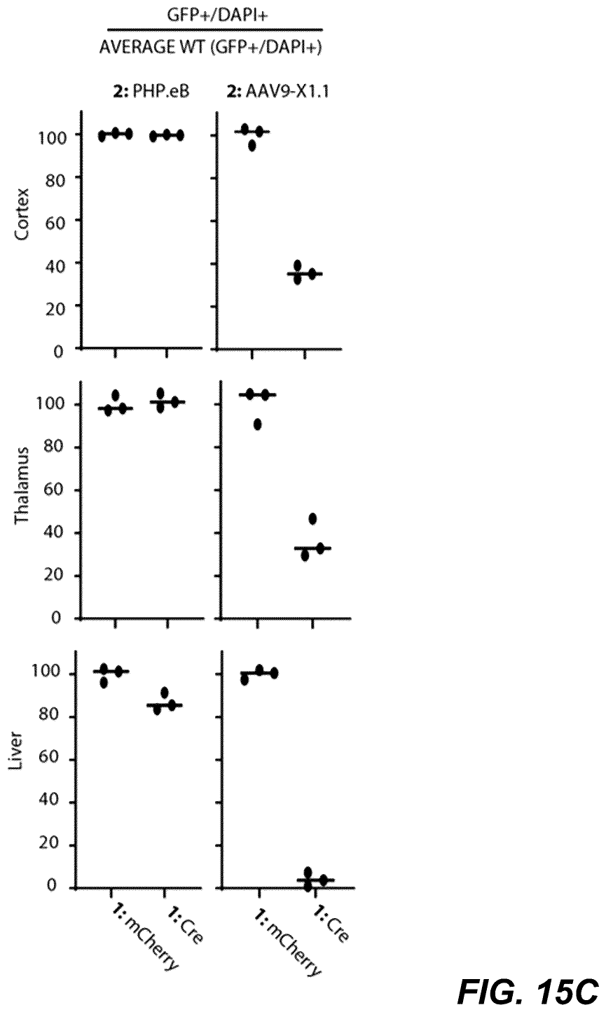

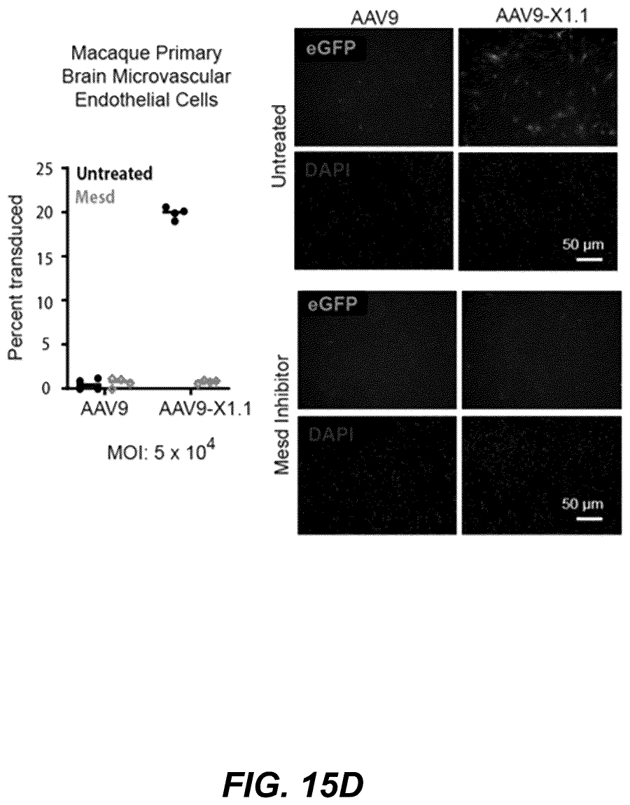

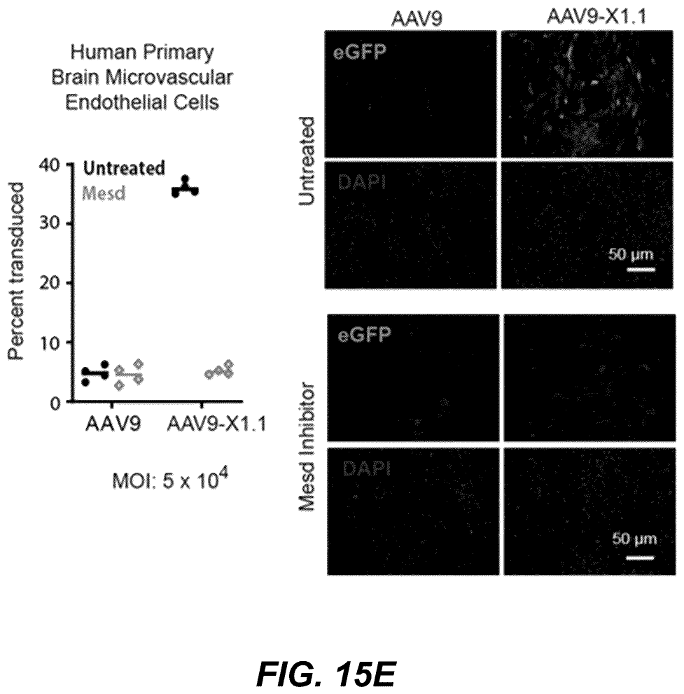

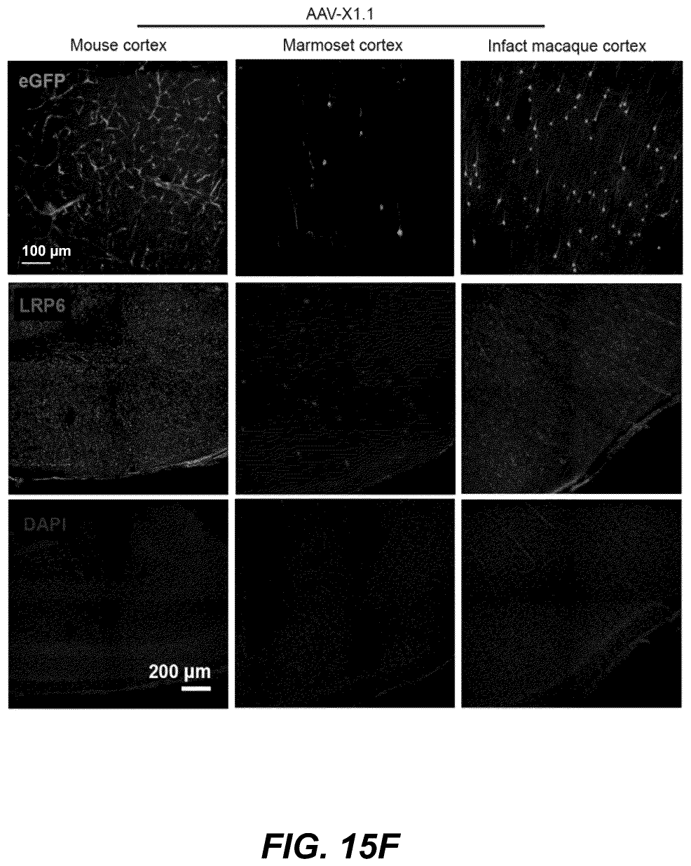

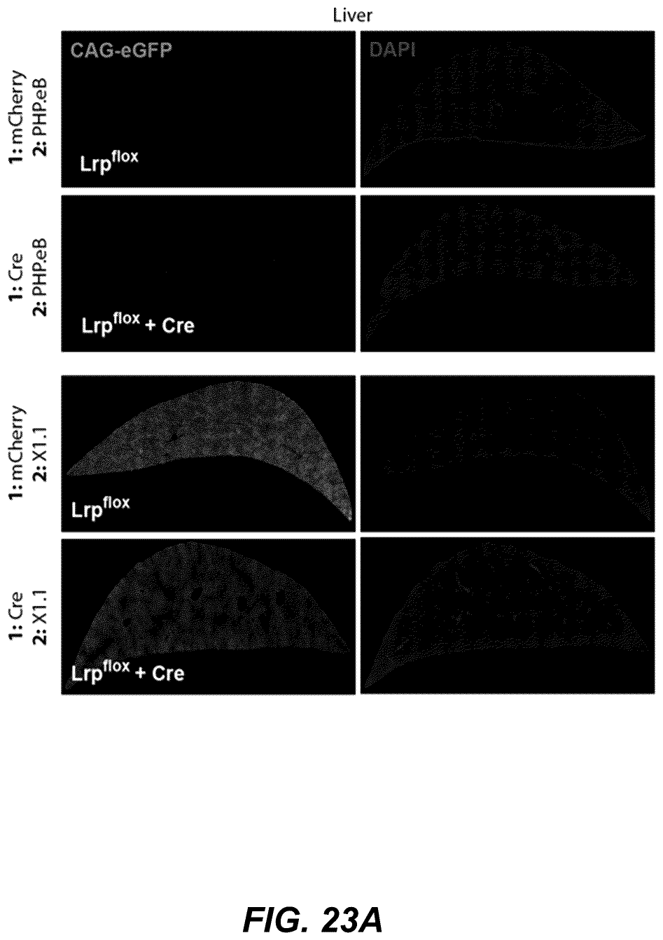

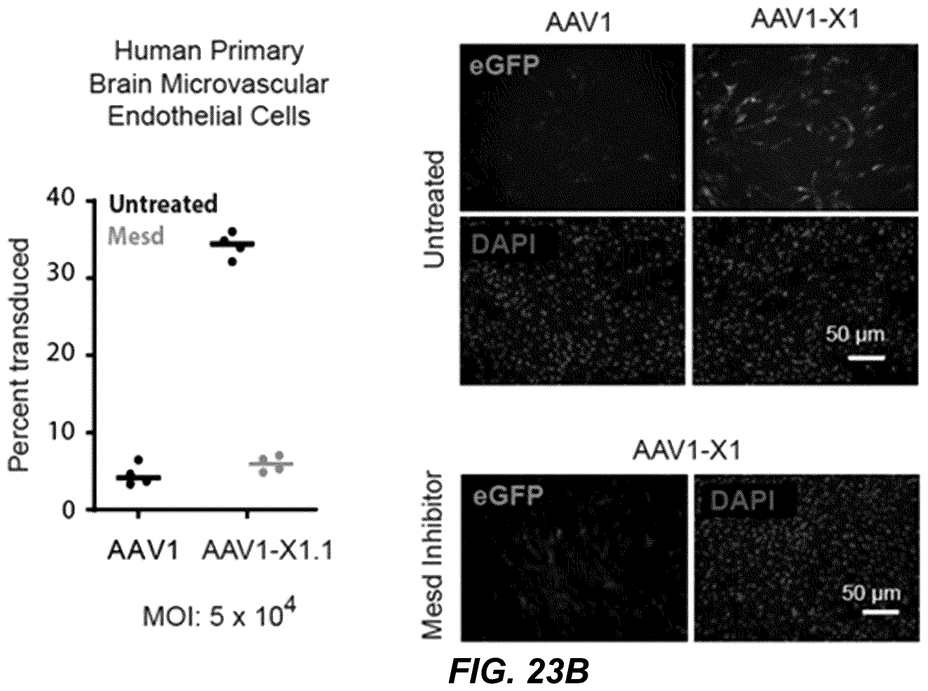

Host neutralizing antibodies, developed in response to prior exposure to AAVs, complicate repeat administration with the same serotypes. Serotype-switched X1 vectors, such as AAV1-X1, were shown to enable a second systemic dosing in mice previously exposed to AAV9-based vectors. This property was leveraged to determine the in vivo effects of AAV9-X1.1's LRP6 interaction (FIG. 15A). Brain endothelium-targeted AAV1-X1 packaging either mCherry as a control or Cre recombinase was systemically administered to Lrp6 Cre-conditional knockout mice. After three weeks, either AAV9-based PHP.eB or AAV9-X1.1 packaging eGFP was systemically delivered. Whereas PHP.eB showed characteristic strong brain transduction regardless of AAV1-X1 cargo, AAV9-X1.1 brain endothelial tropism was markedly reduced in the AAV1-X1-dosed mice with Lrp6 knocked out in AAV1-X1 transfected cells (FIG. 15B-FIG. 15C), confirming LRP6's necessity for capsid BBB entry in vivo. The AAV9-X1.1 capsid showed enhanced potency compared to PHP.eB in the liver, where LRP6 is also expressed (FIG. 23A). In Lrp6 knockout conditions, decreased AAV9-X1.1 liver transduction was also observed (FIG. 15B-FIG. 15C).

To confirm that LRP6 interaction is mediating the brain potency of AAV9-X1.1 in primates, the vector was tested on macaque and human primary brain microvascular endothelial cells (PBMECs) in culture (FIG. 15D-FIG. 15E). AAV9-X1.1 was markedly more potent than its parent, AAV9, in the PBMECs from both species. On the other hand, the LRP6 inhibitor Mesd selectively reduced AAV9-X1.1 potency in PBMECs back to AAV9 levels. A similar LRP6-dependent boost in potency by AAV1-X1 compared to AAV1 was also observed in human PBMECs (FIG. 23B). The similarity of the responses of AAV1-X1 and AAV9-X1.1 supports the SPR experiments (FIG. 20A) that showed the X1 peptide is necessary and sufficient to target capsids to the BBB through its interactions with LRP6.

Recent advances in capsid engineering have led to AAV vectors that can more efficiently cross the blood-brain barrier (BBB) in rodents and non-human primates after systemic administration, but predictable translation and further rational design of these and other non-viral BBB-crossing molecules is hampered by the limited understanding of transcytosis mechanisms, particularly in humans. This translational challenge is also an opportunity to better understand the biology of the BBB and AAV vectors. To date, only a few targets, such as transferrin receptor, are used for research or therapies. Disclosed herein is a pipeline to find cognate receptors for engineered AAVs, focusing on the human membrane proteome and secretome. The present disclosure validates the utility of cell microarray screening to identify receptors for natural and engineered AAVs. Identified herein is LRP6 as a novel and highly conserved target for blood-brain barrier transcytosis by AAV9-X1.1, a potent engineered capsid for primate neurons and human IL3 as an interaction partner for AAV9. These findings enable the leveraging of identified receptors for targeted drug delivery across diverse therapeutic modalities, such as small molecules, antibodies, or oligonucleotides.

Delivery vector safety and immune tolerance are key considerations with AAVs moving into the clinic as adverse reactions can occur. The immunomodulatory potential of the IL3-AAV9 interaction can include providing a host neutralization mechanism, or a cloaking mechanism for the AAV to evade the immune system or use decoy receptors to weaken its response. In addition to activated T cells, IL3 is also constitutively secreted by astrocytes in the brain to reprogram microglia, and combat Alzheimer's disease. Thus, AAV9 interaction with IL3, which is shared with all AAV9-based engineered capsids for enhanced BBB crossing, may, in some embodiments, impact processes beyond immune tolerance of the vector itself in the context of healthy and diseased brains. Importantly, AAV9, an isolate from human clinical tissue, binds to human and macaque (83% AA identity) but not marmoset or mouse IL3 (69% and 27% AA identity, respectively). Without being bound by any particular theory, this species-dependent interaction may contribute to a disconnect between rodent and primate AAV safety profiles, especially in neurodegeneration contexts.

The high degree of sequence conservation in LRP6 (98% AA identity between mouse and human) can, without being bound by any particular theory, explain the broad conservation across species of enhanced tropism by AAV capsids targeting this receptor.

That both AAV9-X1.1 and CAP-Mac also showed binding to GP2, which is not present in the CNS, suggests that the interaction, without being bound by any particular theory, may have piggybacked on the functional enhancement provided by LRP6-binding during directed evolution selections. This is supported by the finding that the AAVs more potently interact with human GP2 than the mouse protein that was present during the directed evolution of AAV9-X1.1. These findings highlight the importance of broad, unbiased interaction screens as disclosed herein to build full safety profiles for engineered capsids prior to clinical trials.

Surveying the diversity of mechanisms by which natural and engineered AAVs cross the BBB may also allow for preparation in defense against future pathogens. Just as antibiotic resistance is testing the modern world, one concern is that fast-evolving pathogens will develop “BBB resistance”—the ability to access the brain and cause severe disease (as some retroviruses, including HIV-1, already do). As a recent troubling example, SARS-CoV-2 capsid proteins were found in the brains of patients with long COVID, and correlated with neuropsychiatric symptoms. By screening existing pathogens and their likely molecular evolutions against the growing human BBB transcytosis receptor catalog (including transferrin receptor, insulin receptor, CD98hc, CA4, and the presently disclosed LRP6, outbreaks of pathogens with neuropsychiatric sequelae can be anticipated.

Provided herein are methods to efficiently screen natural AAV serotypes and engineered variants against the human proteome. The present disclosure expands the limited roster of targets for enhanced BBB crossing in primates. These findings suggest new strategies for successful clinical translation of engineered AAVs, provide targets for development of non-viral therapeutic modalities, and highlight latent vulnerabilities to future pathogens.

TABLE 3
CAPSID ENGINEERING DETAILS AND IN VIVO TROPISMS OF VECTORS
USED IN THIS STUDY

| | | | | Enhanced systemic tropism | | |
|---|---|---|---|---|---|---|
| | | | | in the nervous system | | |
| | AAV9 modified sequence | | Selection | | Adult | Infant |
| Capsid name | VR IV | VR VIII | species | Adult Mouse | Marmoset | Macaque |
| AAV9 | NGSGQNQ | AQ------AQ | | | | |
| (parent of | (SEQ ID NO: 1) | (SEQ ID NO: 2) | | | | |
| vectors below) | | | | | | |
| AAV.CAP-B10 | DGAATKN | DGTLAVPFKAQ | Mouse | CNS | CNS | |
| | (SEQ ID NO: 3) | (SEQ ID NO: 4) | | (neuronal) | (neuronal) | |
| AAV.CAP-B22 | DGQSSKS (SEQ | DGTLAVPFKAQ | Mouse | CNS | CNS | |
| | ID NO: 5) | (SEQ ID NO: 4) | | | (neuronal & | |
| | | | | | astrocytic) | |
| AAV- | | AQPHEGSSRAQ | Mouse | PNS | CNS & PNS | CNS & PNS |
| MaCPNS1 | | (SEQ ID NO: 6) | | | (neuronal & | (neuronal & |
| | | | | | astrocytic) | astrocytic) |
| AAV- | | AQPNASVNSAQ | Mouse | PNS | CNS & PNS | CNS & PNS |
| MaCPNS2 | | (SEQ ID NO: 7) | | | (neuronal & | (neuronal & |
| | | | | | astrocytic) | astrocytic) |
| AAV.CAP-Mac | | AQLNTTKPIAQ | Marmoset | CNS (weak | CNS | CNS |
| | | (SEQ ID NO: 8) | | endothelial) | (endothelial) | (neuronal) |
| AAV9-X1.1 | DGAATKN | AQGNNTRSVAQ | Mouse | CNS | CNS | CNS |
| | (SEQ ID NO: 3) | (SEQ ID NO: 9) | | (endothelial) | (weak | (neuronal) |
| | | | | | neuronal) | |
| AAV-BI30 | | AQNNSTRGGAQ | Mouse | CNS | | |
| | | (SEQ ID NO: 10) | | (endothelial) | | |

TABLE 4
SUMMARY OF IDENTIFIED AAV INTERACTIONS. AAV9 HITS
WERE OBSERVED IN ALL ENGINEERED DERIVATIVES

| | |
|---|---|
| Screen hits | Validated |

| Vector name | Membrane | Secreted | interactions |
|---|---|---|---|
| AAV9 | DPP4 | DKK3 | IL3 |
| | | IL3 | |
| AAV.CAP-B22 | LY6A | | LY6A |
| | FAM234A | | FAM234A |
| AAV-MaCPNS1 | | | |
| AAV-MacCPNS2 | | | |
| AAV.CAP-Mac | GP2 | | LRP6 |
| | | | GP2 |
| AAV9-X1.1 | GP2 | CSF2 | LRP6 |
| | LRP6 | EPYC | GP2 |
| | ANPEP | | |

TABLE 5
DOSES OF INDIVIDUAL VECTORS WITHIN THE
POOLS USED TO OPTIMIZE SIGNAL TO NOISE

| | Pool dose (v.g./cell) | | |
|---|---|---|---|
| Vector name | 7.9 × 10⁵ | 5.1 × 10⁵ | 1.6 × 10⁵ |
| AAV9 | 2 × 10⁵ | 1 × 10⁵ | 3 × 10⁴ |
| AAV.CAP-B22 | 6 × 10⁴ | 1 × 10⁵ | 3 × 10⁴ |
| AAV-MaCPNS1 | 1.5 × 10⁵ | 1 × 10⁵ | 3 × 10⁴ |
| AAV-MaCPNS2 | 1.5 × 10⁵ | 1 × 10⁵ | 3 × 10⁴ |
| AAV.CAP-Mac | 2 × 10⁵ | 1 × 10⁵ | 3 × 10⁴ |
| AAV9-X1.1 | 3 × 10⁴ | 1 × 10⁴ | 1 × 10⁴ |

Table 6 below displays amino acid sequences of exemplary targets of AAVs identified herein. Table 7 shows binding site residues on human LRP6.

TABLE 6
AMINO ACID SEQUENCES OF EXEMPLARY TARGETS IDENTIFIED

| | SEQ ID | |
|---|---|---|
| Name* | NO: | Sequence |
| hDPP4 | 19 | MKTPWKVLLGLLGAAALVTIITVPVVLLNKGTDDATADSRKTYTLTDY |
| | | LKNTYRLKLYSLRWISDHEYLYKQENNILVFNAEYGNSSVFLENSTFDE |
| | | FGHSINDYSISPDGQFILLEYNYVKQWRHSYTASYDIYDLNKRQLITEER |
| | | IPNNTQWVTWSPVGHKLAYVWNNDIYVKIEPNLPSYRITWTGKEDIIYN |
| | | GITDWVYEEEVFSAYSALWWSPNGTFLAYAQFNDTEVPLIEYSFYSDES |
| | | LQYPKTVRVPYPKAGAVNPTVKFFVVNTDSLSSVTNATSIQITAPASMLI |
| | | GDHYLCDVTWATQERISLQWLRRIQNYSVMDICDYDESSGRWNCLVA |
| | | RQHIEMSTTGWVGRFRPSEPHFTLDGNSFYKIISNEEGYRHICYFQIDKK |
| | | DCTFITKGTWEVIGIEALTSDYLYYISNEYKGMPGGRNLYKIQLSDYTK |
| | | VTCLSCELNPERCQYYSVSFSKEAKYYQLRCSGPGLPLYTLHSSVNDKG |
| | | LRVLEDNSALDKMLQNVQMPSKKLDFIILNETKFWYQMILPPHFDKSK |
| | | KYPLLLDVYAGPCSQKADTVFRLNWATYLASTENIIVASFDGRGSGYQ |
| | | GDKIMHAINRRLGTFEVEDQIEAARQFSKMGFVDNKRIAIWGWSYGGY |
| | | VTSMVLGSGSGVFKCGIAVAPVSRWEYYDSVYTERYMGLPTPEDNLDH |
| | | YRNSTVMSRAENFKQVEYLLIHGTADDNVHFQQSAQISKALVDVGVDF |
| | | QAMWYTDEDHGIASSTAHQHIYTHMSHFIKQCFSLP |
| hDKK3 | 20 | MQRLGATLLCLLLAAAVPTAPAPAPTATSAPVKPGPALSYPQEEATLNE |
| | | MFREVEELMEDTQHKLRSAVEEMEAEEAAAKASSEVNLANLPPSYHNE |
| | | TNTDTKVGNNTIHVHREIHKITNNQTGQMVFSETVITSVGDEEGRRSHE |
| | | CIIDEDCGPSMYCQFASFQYTCQPCRGQRMLCTRDSECCGDQLCVWGH |
| | | CTKMATRGSNGTICDNQRDCQPGLCCAFQRGLLFPVCTPLPVEGELCH |
| | | DPASRLLDLITWELEPDGALDRCPCASGLLCQPHSHSLVYVCKPTFVGS |
| | | RDQDGEILLPREVPDEYEVGSFMEEVRQELEDLERSLTEEMALREPAAA |
| | | AAALLGGEEI |
| mIL3 | 21 | MVLASSTTSIHTMLLLLLMLFHLGLQASISGRDTHRLTRTLNCSSIVKEII |
| | | GKLPEPELKTDDEGPSLRNKSFRRVNLSKFVESQGEVDPEDRYVIKSNL |
| | | QKLNCCLPTSANDSALPGVFIRDLDDFRKKLRFYMVHLNDLETVLTSRP |
| | | PQPASGSVSPNRGTVEC |
| nhpIL3 | 22 | MSRLPVLLLLHLLVSPGLQAPMTQTTSLKTSWAKCSNMIDEIITHLNQPP |
| | | LPSPDFNNLNEEDQTILVEKNLRRSNLEAFSKAVKSLQNASAIESILKNLP |
| | | PCLPMATAAPTRPPIRITNGDRNDFRRKLKFYLKTLENEQAQ |
| hIL3 | 23 | MSRLPVLLLLQLLVRPGLQAPMTQTTPLKTSWVNCSNMIDEIITHLKQP |
| | | PLPLLDFNNLNGEDQDILMENNLRRPNLEAFNRAVKSLQNASAIESILKN |
| | | LLPCLPLATAAPTRHPIHIKDGDWNEFRRKLTFYLKTLENAQAQQTTLS |
| | | LAIF |
| mFAM234A | 24 | MMDNKDLEAEIHPLKNEDKKSQENPGNLPRNEDNLKSKPVPSRLSRCR |
| | | TVAFFLSLFTCLFVVFVLSFIIPCPDRPSSQGTWKLDYNNAVMYDFLALG |
| | | DINKDKVQDVLFLYKNTNSSNNLTRSCADEGFSTPCAFVVAVSGANGS |
| | | VLWERPVAQDVALVKCAMPQTLDSDEVSSACIVVGRAGSFVAVSFFTG |
| | | ETLWSHPSSFSGNVSILSPLLQVPDIDGDGDGTPDLLILAQEGQEVSGAL |
| | | YSGSTGYQIGHRGSLGVDGDGVALLHVTRTGAQYILLPCASALCGFSV |
| | | KSLYERITGRDGHFKEDPYWENMLNHSVHRRLLHRLGAVRYLMNIPGK |
| | | AGQDLLLVTSEACVLLDGQDLEPRWTLGEVQVLRKPILGHYKPDTLAV |
| | | VIENGTSIDRQILLLDLSTGSILWSQPLPSLPGGPPSTSLMTADHRSAFFF |
| | | WGLHDLVSTNEMDPPDVQHSLYMFHPTLPGILLELANVSANIVAFDAV |
| | | LLEPSRHAAYVLLTGPASSDVPGLVSVTKHKVQDLVPGSRVIHLGEGSS |
| | | DSDQAIRDRFSRLRYRSEM |
| hFAM234A | 25 | MMDNKDLEAEIHPLKNEDKKSQENPGNLPRNEDNLKSKPVPSRLSRCR |
| | | TVAFFLSLFTCLFVVFVLSFIIPCPDRPSSQGTWKLDYNNAVMYDFLALG |
| | | DINKDKVQDVLFLYKNTNSSNNLTRSCADEGFSTPCAFVVAVSGANGS |
| | | VLWERPVAQDVALVKCAMPQTLDSDEVSSACIVVGRAGSFVAVSFFTG |
| | | ETLWSHPSSFSGNVSILSPLLQVPDIDGDGDGTPDLLILAQEGQEVSGAL |
| | | YSGSTGYQIGHRGSLGVDGDGVALLHVTRTGAQYILLPCASALCGFSV |
| | | KSLYERITGRDGHFKEDPYWENMLNHSVHRRLLHRLGAVRYLMNIPGK |
| | | AGQDLLLVTSEACVLLDGQDLEPRWTLGEVQVLRKPILGHYKPDTLAV |
| | | VIENGTSIDRQILLLDLSTGSILWSQPLPSLPGGPPSTSLMTADHRSAFFF |
| | | WGLHDLVSTNEMDPPDVQHSLYMFHPTLPGILLELANVSANIVAFDAV |
| | | LLEPSRHAAYVLLTGPASSDVPGLVSVTKHKVQDLVPGSRVIHLGEGSS |
| | | DSDQAIRDRFSRLRYRSEM |
| mGP2 | 26 | MKRMVGCDLLWLAAASCVLTLVSPSTIHQGYGRPRNSSNLDLDCGSPD |
| | | SPSSGICFDPCQNHTVLNDPTRSTENNDSSVAWCDDNLHGWYRFVGDG |
| | | GVKMPETCVSVFRCHTSAPMWLSGSHPILGDGIVSHTACANWNENCCF |
| | | WRSEVQVKACSEELGEYHVYKLQGTPECSLRYCTDPSTAPKNCEITCRP |
| | | EEECVFQNNNWSCVCRQDLHVSDSQSLQPLLDCGDNEIKVKLDKCLLG |
| | | GMGFKEEIIAYLNDRNCNGTMQDEPNNWVSMTSPVVANYCGNILEKN |
| | | GTHAIYRNTLSLATDFIIRDFRVNVNFQCAYPLDMSVSLETALQPIVSSL |
| | | TVDVDGAGEFNVKMALFQDQSYTNPYEGAEVLLPVESILYVGVLLNRG |
| | | DTSRFKLLLTNCYATPSEDRHDPVKYFIIKNRCPNQRDSTINVRENGVSS |
| | | ESRFSVQMFMFAGNYDLVFLHCEVYLCDSTTEQCQPSCSTNRLRSSRPA |
| | | IDYNRVLDLGPITKRSAQSSATSKGTPHTTGFLLAWPMFFLPVFLALLF |
| nhpGP2 | 27 | MPHLMERIVGSGLLWLALVSCILAQASAVQQGYGNPSEASSYGLDLDC |
| | | GAPGTPEAHICFDPCQNYTLLDEPFRSTENSEEKEGCDDNMSGWYRFA |
| | | GEGGVRMSETCVQVHRCQTAAPMWLNGTHPALGDGIVNRTACAHWS |
| | | GNCCLWKTEVLVKACPGGYHVYRLEGTPECSLRYCTDPSTVEDKCEKA |
| | | CRPEEECLARNSTWGCFCRQDLNSSDFHSLQPQLDCGAEEIKVKVDKCL |
| | | LGGLGLEEEVIAYLRDPNCSSILQTGETNWVSVTSPVQASACRNILERNQ |
| | | THAIYKNTLSLVSDFIIRDTILNINFQCAYPLDMKVSLQAALKPIVSFLNV |
| | | SVGGDGEFIVRMALFQDQNYTNPYEGDVAELSVESMLYVGAILEQGDT |
| | | SRFNLVLRNCYATPTEDKADPVKYFIIRNSCSNQRDSTIHVEENGRSSES |
| | | RFSVQMFMFAGHYDLVFLHCEIHLCDSFNEQCQPSCPRSQVRSEVPAID |
| | | LSRVLDLGPITRRGAQSPGVMNGTPGTAGFLVAWPMVLLTVLLAWLF |
| hGP2 | 28 | MPHLMERMVGSGLLWLALVSCILTQASAVQRGYGNPIEASSYGLDLDC |
| | | GAPGTPEAHVCFDPCQNYTLLDEPFRSTENSAGSQGCDKNMSGWYRFV |
| | | GEGGVRMSETCVQVHRCQTDAPMWLNGTHPALGDGITNHTACAHWS |
| | | GNCCFWKTEVLVKACPGGYHVYRLEGTPWCNLRYCTVPRDPSTVEDK |
| | | CEKACRPEEECLALNSTWGCFCRQDLNSSDVHSLQPQLDCGPREIKVKV |
| | | DKCLLGGLGLGEEVIAYLRDPNCSSILQTEERNWVSVTSPVQASACRNI |
| | | LERNQTHAIYKNTLSLVNDFIIRDTILNINFQCAYPLDMKVSLQAALQPI |
| | | VSSLNVSVDGNGEFIVRMALFQDQNYTNPYEGDAVELSVESVLYVGAI |
| | | LEQGDTSRFNLVLRNCYATPTEDKADLVKYFIIRNSCSNQRDSTIHVEEN |
| | | GQSSESRFSVQMFMFAGHYDLVFLHCEIHLCDSLNEQCQPSCSRSQVRS |
| | | EVPAIDLARVLDLGPITRRGAQSPGVMNGTPSTAGFLVAWPMVLLTVL |
| | | LAWLF |
| mLRP6 | 29 | MGAVLRSLLACSFCVLLRAAPLLLYANRRDLRLVDATNGKENATIVVG |
| | | GLEDAAAVDFVFGHGLIYWSDVSEEAIKRTEFNKSESVQNVVVSGLLSP |
| | | DGLACDWLGEKLYWTDSETNRIEVSNLDGSLRKVLFWQELDQPRAIAL |
| | | DPSSGFMYWTDWGEVPKIERAGMDGSSRFVIINTEIYWPNGLTLDYQER |
| | | KLYWADAKLNFIHKSNLDGTNRQAVVKGSLPHPFALTLFEDTLYWTD |
| | | WNTHSILACNKYTGEGLREIHSNIFSPMDIHAFSQQRQPNATNPCGIDNG |
| | | GCSHLCLMSPVKPFYQCACPTGVKLMENGKTCKDGATELLLLARRTDL |
| | | RRISLDTPDFTDIVLQLEDIRHAIAIDYDPVEGYIYWTDDEVRAIRRSFID |
| | | GSGSQFVVTAQIAHPDGIAVDWVARNLYWTDTGTDRIEVTRLNGTMR |
| | | KILISEDLEEPRAIVLDPMVGYMYWTDWGEIPKIERAALDGSDRVVLVN |
| | | TSLGWPNGLALDYDEGTIYWGDAKTDKIEVMNTDGTGRRVLVEDKIPH |
| | | IFGFTLLGDYVYWTDWQRRSIERVHKRSAEREVIIDQLPDLMGLKATSV |
| | | HRVIGSNPCAEDNGGCSHLCLYRPQGLRCACPIGFELIGDMKTCIVPEAF |
| | | LLFSRRADIRRISLETNNNNVAIPLTGVKEASALDFDVTDNRIYWTDISL |
| | | KTISRAFMNGSALEHVVEFGLDYPEGMAVDWLGKNLYWADTGTNRIE |
| | | VSKLDGQHRQVLVWKDLDSPRALALDPAEGFMYWTEWGGKPKIDRA |
| | | AMDGSERTTLVPNVGRANGLTIDYAKRRLYWTDLDTNLIESSDMLGLN |
| | | REVIADDLPHPFGLTQYQDYIYWTDWSRRSIERANKTSGQNRTIIQGHL |
| | | DYVMDILVFHSSRQAGWNECASSNGHCSHLCLAVPVGGFVCGCPAHY |
| | | SLNADNRTCSAPSTFLLFSQKSAINRMVIDEQQSPDIILPIHSLRNVRAID |
| | | YDPLDKQLYWIDSRQNSIRKAHEDGGQGFNVVANSVANQNLEIQPYDL |
| | | SIDIYSRYIYWTCEATNVIDVTRLDGRSVGVVLKGEQDRPRAIVVNPEK |
| | | GYMYFTNLQERSPKIERAALDGTEREVLFFSGLSKPIALALDSKLGKLF |
| | | WADSDLRRIESSDLSGANRIVLEDSNILQPVGLTVFENWLYWIDKQQQ |
| | | MIEKIDMTGREGRTKVQARIAQLSDIHAVKELNLQEYRQHPCAQDNGG |
| | | CSHICLVKGDGTTRCSCPMHLVLLQDELSCGEPPTCSPQQFTCFTGDIDC |
| | | IPVAWRCDGFTECEDHSDELNCPVCSESQFQCASGQCIDGALRCNGDA |
| | | NCQDKSDEKNCEVLCLIDQFRCANGQCVGKHKKCDHSVDCSDRSDEL |
| | | DCYPTEEPAPQATNTVGSVIGVIVTIFVSGTIYFICQRMLCPRMKGDGET |
| | | MTNDYVVHSPASVPLGYVPHPSSLSGSLPGMSRGKSMISSLSIMGGSSG |
| | | PPYDRAHVTGASSSSSSSTKGTYFPAILNPPPSPATERSHYTMEFGYSSNS |
| | | PSTHRSYSYRPYSYRHFAPPTTPCSTDVCDSDYAPSRRMTSVATAKGYT |
| | | SDVNYDSEPVPPPPTPRSQYLSAEENYESCPPSPYTERSYSHHLYPPPPSP |
| | | CTDSS |
| nhpLRP6 | 30 | MGAVLRSLLACSFCVLLRAAPLLLYANRRDLRLVDATNGKENATIVVG |
| | | GLEDAAAVDFVFGHGLIYWSDVSEEAIKRTEFNKTESVQNVVVSGLLSP |
| | | DGLACDWLGEKLYWTDSETNRIEVSNLDGSLRKVLFWQELDQPRAIAL |
| | | DPSSGFMYWTDWGEVPKIERAGMDGSSRFIIINSEIYWPNGLTLDYEEQ |
| | | KLYWADAKLNFIHKSNLDGTNRQAVVKGSLPHPFALTLFEDILYWTDW |
| | | STHSILACNKYTGEGLREIHSNIFSPMDIHAFSQQRQPNATNPCGIDNGG |
| | | CSHLCLMSPVKPFYQCACPTGVKLLENGKTCKDGATELLLLARRTDLR |
| | | RISLDTPDFTDIVLQLEDIRHAIAIDYDPVEGYIYWTDDEVRAIRRSFIDG |
| | | SGSQFVVTAQIAHPDGIAVDWVARNLYWTDTGTDRIEVTRLNGTMRKI |
| | | LISEDLEEPRAIVLDPMVGYMYWTDWGEIPKIERAALDGSDRVVLVNTS |
| | | LGWPNGLALDYDEGKIYWGDAKTDKIEVMNTDGTGRRVLVEDKIPHIF |
| | | GFTLLGDYVYWTDWQRRSIERVHKRSAEREVIIDQLPDLMGLKATNVH |
| | | RVIGSNPCAEENGGCSHLCLYRPQGLRCACPIGFELISDMKTCIVPEAFL |
| | | LFSRRADIRRISLETNNNNVAIPLTGVKEASALDFDVTDNRIYWTDISLK |
| | | TISRAFMNGSALEHVVEFGLDYPEGMAVDWLGKNLYWADTGTNRIEV |
| | | SKLDGQHRQVLVWKDLDSPRALALDPAEGFMYWTEWGGKPKIDRAA |
| | | MDGSERTTLVPNVGRANGLTIDYAKRRLYWTDLDTNLIESSNMLGLNR |
| | | EVIADDLPHPFGLTQYQDYIYWTDWSRRSIERANKTSGQNRTIIQGHLD |
| | | YVMDILVFHSSRQSGWNECASSNGHCSHLCLAVPVGGFVCGCPAHYAL |
| | | NADNRTCSAPTTFLLFSQKSAINRMVIDEQQSPDIILPIHSLRNVRAIDYD |
| | | PLDKQLYWIDSRQNMIRKAQEDGSQGFTVVVSSVPSQNLEIQPYDLSIDI |
| | | YSRYIYWTCEATNVINVTRLDGRSIGVVLKGEQDRPRAIVVNPEKGYM |
| | | YFTNLQERSPKIERAALDGTEREVLFFSGLSKPIALALDSRLGKLFWADS |
| | | DLRRIESSDLSGANRIVLEDSNILQPVGLTVFENWLYWIDKQQQMIEKID |
| | | MTGREGRTKVQARIAQLSDIHAVKELNLQEYRQHPCAQDNGGCSHICL |
| | | VKGDGTTRCSCPMHLVLLQDELSCGEPPTCSPQQFTCFTGEIDCIPVAW |
| | | RCDGFTECEDHSDELNCPVCSESQFQCASGQCIDGALRCNGDANCQDK |
| | | SDEKNCEVLCLIDQFRCANGQCIGKHKKCDHNVDCSDKSDELDCYPTE |
| | | EPAPQATNTVGSVIGVIVTIFVSGTIYFICQRMLCPRMKGDGETMINDY |
| | | VVHGPASVPLGYVPHPSSLSGSLPGMSRGKSMISSLSIMGGSSGPPYDRA |
| | | HVTGASSSSSSSTKGTYFPAILNPPPSPATERSHYTMEFGYSSNSPSTHRS |
| | | YSYRPYSYRHFAPPTTPCSTDVCDSDYAPSRRMTSVATAKGYTSDLNY |
| | | DSEPVPPPPTPRSQYLSAEENYESCPPSPYTERSYSHHLYPPPPSPCTDSS |
| hLRP6 | 31 | MGAVLRSLLACSFCVLLRAAPLLLYANRRDLRLVDATNGKENATIVVG |
| | | GLEDAAAVDFVFSHGLIYWSDVSEEAIKRTEFNKTESVQNVVVSGLLSP |
| | | DGLACDWLGEKLYWTDSETNRIEVSNLDGSLRKVLFWQELDQPRAIAL |
| | | DPSSGFMYWTDWGEVPKIERAGMDGSSRFIIINSEIYWPNGLTLDYEEQ |
| | | KLYWADAKLNFIHKSNLDGTNRQAVVKGSLPHPFALTLFEDILYWTDW |
| | | STHSILACNKYTGEGLREIHSDIFSPMDIHAFSQQRQPNATNPCGIDNGG |
| | | CSHLCLMSPVKPFYQCACPTGVKLLENGKTCKDGATELLLLARRTDLR |
| | | RISLDTPDFTDIVLQLEDIRHAIAIDYDPVEGYIYWTDDEVRAIRRSFIDG |
| | | SGSQFVVTAQIAHPDGIAVDWVARNLYWTDTGTDRIEVTRLNGTMRKI |
| | | LISEDLEEPRAIVLDPMVGYMYWTDWGEIPKIERAALDGSDRVVLVNTS |
| | | LGWPNGLALDYDEGKIYWGDAKTDKIEVMNTDGTGRRVLVEDKIPHIF |
| | | GFTLLGDYVYWTDWQRRSIERVHKRSAEREVIIDQLPDLMGLKATNVH |
| | | RVIGSNPCAEENGGCSHLCLYRPQGLRCACPIGFELISDMKTCIVPEAFL |
| | | LFSRRADIRRISLETNNNNVAIPLTGVKEASALDFDVTDNRIYWTDISLK |
| | | TISRAFMNGSALEHVVEFGLDYPEGMAVDWLGKNLYWADTGTNRIEV |
| | | SKLDGQHRQVLVWKDLDSPRALALDPAEGFMYWTEWGGKPKIDRAA |
| | | MDGSERTTLVPNVGRANGLTIDYAKRRLYWTDLDTNLIESSNMLGLNR |
| | | EVIADDLPHPFGLTQYQDYIYWTDWSRRSIERANKTSGQNRTIIQGHLD |
| | | YVMDILVFHSSRQSGWNECASSNGHCSHLCLAVPVGGFVCGCPAHYSL |
| | | NADNRTCSAPTTFLLFSQKSAINRMVIDEQQSPDIILPIHSLRNVRAIDYD |
| | | PLDKQLYWIDSRQNMIRKAQEDGSQGFTVVVSSVPSQNLEIQPYDLSIDI |
| | | YSRYIYWTCEATNVINVTRLDGRSVGVVLKGEQDRPRAVVVNPEKGY |
| | | MYFTNLQERSPKIERAALDGTEREVLFFSGLSKPIALALDSRLGKLFWA |
| | | DSDLRRIESSDLSGANRIVLEDSNILQPVGLTVFENWLYWIDKQQQMIE |
| | | KIDMTGREGRTKVQARIAQLSDIHAVKELNLQEYRQHPCAQDNGGCSH |
| | | ICLVKGDGTTRCSCPMHLVLLQDELSCGEPPTCSPQQFTCFTGEIDCIPV |
| | | AWRCDGFTECEDHSDELNCPVCSESQFQCASGQCIDGALRCNGDANCQ |
| | | DKSDEKNCEVLCLIDQFRCANGQCIGKHKKCDHNVDCSDKSDELDCYP |
| | | TEEPAPQATNTVGSVIGVIVTIFVSGTVYFICQRMLCPRMKGDGETMTN |
| | | DYVVHGPASVPLGYVPHPSSLSGSLPGMSRGKSMISSLSIMGGSSGPPYD |
| | | RAHVTGASSSSSSSTKGTYFPAILNPPPSPATERSHYTMEFGYSSNSPSTH |
| | | RSYSYRPYSYRHFAPPTTPCSTDVCDSDYAPSRRMTSVATAKGYTSDLN |
| | | YDSEPVPPPPTPRSQYLSAEENYESCPPSPYTERSYSHHLYPPPPSPCTDS |
| | | S |
| hANPEP | 32 | MAKGFYISKSLGILGILLGVAAVCTIIALSVVYSQEKNKNANSSPVASTT |
| | | PSASATTNPASATTLDQSKAWNRYRLPNTLKPDSYRVTLRPYLTPNDRG |
| | | LYVFKGSSTVRFTCKEATDVIIIHSKKLNYTLSQGHRVVLRGVGGSQPP |
| | | DIDKTELVEPTEYLVVHLKGSLVKDSQYEMDSEFEGELADDLAGFYRSE |
| | | YMEGNVRKVVATTQMQAADARKSFPCFDEPAMKAEFNITLIHPKDLTA |
| | | LSNMLPKGPSTPLPEDPNWNVTEFHTTPKMSTYLLAFIVSEFDYVEKQA |
| | | SNGVLIRIWARPSAIAAGHGDYALNVTGPILNFFAGHYDTPYPLPKSDQI |
| | | GLPDFNAGAMENWGLVTYRENSLLFDPLSSSSSNKERVVTVIAHELAH |
| | | QWFGNLVTIEWWNDLWLNEGFASYVEYLGADYAEPTWNLKDLMVLN |
| | | DVYRVMAVDALASSHPLSTPASEINTPAQISELFDAISYSKGASVLRMLS |
| | | SFLSEDVFKQGLASYLHTFAYQNTIYLNLWDHLQEAVNNRSIQLPTTVR |
| | | DIMNRWTLQMGFPVITVDTSTGTLSQEHFLLDPDSNVTRPSEFNYVWIV |
| | | PITSIRDGRQQQDYWLIDVRAQNDLFSTSGNEWVLLNLNVTGYYRVNY |
| | | DEENWRKIQTQLQRDHSAIPVINRAQIINDAFNLASAHKVPVTLALNNT |
| | | LFLIEERQYMPWEAALSSLSYFKLMFDRSEVYGPMKNYLKKQVTPLFIH |
| | | FRNNTNNWREIPENLMDQYSEVNAISTACSNGVPECEEMVSGLFKQWM |
| | | ENPNNNPIHPNLRSTVYCNAIAQGGEEEWDFAWEQFRNATLVNEADKL |
| | | RAALACSKELWILNRYLSYTLNPDLIRKQDATSTIISITNNVIGQGLVWD |
| | | FVQSNWKKLFNDYGGGSFSFSNLIQAVTRRFSTEYELQQLEQFKKDNEE |
| | | TGFGSGTRALEQALEKTKANIKWVKENKEVVLQWFTENSK |
| hCSF2 | 33 | MWLQSLLLLGTVACSISAPARSPSPSTQPWEHVNAIQEARRLLNLSRDT |
| | | AAEMNETVEVISEMFDLQEPTCLQTRLELYKQGLRGSLTKLKGPLTMM |
| | | ASHYKQHCPPTPETSCATQIITFESFKENLKDFLLVIPFDCWEPVQE |
| hEPYC | 34 | MKTLAGLVLGLVIFDAAVTAPTLESINYDSETYDATLEDLDNLYNYENI |
| | | PVDKVEIEIATVMPSGNRELLTPPPQPEKAQEEEEEEESTPRLIDGSSPQE |
| | | PEFTGVLGPHTNEDFPTCLLCTCISTTVYCDDHELDAIPPLPKNTAYFYS |
| | | RFNRIKKINKNDFASLSDLKRIDLTSNLISEIDEDAFRKLPQLRELVLRDN |
| | | KIRQLPELPTTLTFIDISNNRLGRKGIKQEAFKDMYDLHHLYLTDNNLDH |
| | | IPLPLPENLRALHLQNNNILEMHEDTFCNVKNLTYIRKALEDIRLDGNPI |
| | | NLSKTPQAYMCLPRLPVGSLV |

*m, mouse; nhp, Rhesus Macaque; h, human

TABLE 7
HUMAN LRP6 E1 DOMAIN BINDING SITE RESIDUES

| AAV.CAP-Mac | AAV9-X1.1 |
|---|---|
| R28 | S96 |
| G158 | S114 |
| E159 | E115 |
| W183 | R141 |
| A201 | W157 |
| K202 | W183 |
| H226 | W242 |

LRP6 amino acid side chains within 3 angstroms or main chains within 5 angstroms of engineered AAV-derived peptide

### Methods

Viral Vector Production

Briefly, HEK293 cells were triple transfected with capsid, genome, and helper plasmids. Media was exchanged the next day then collected and replaced two days after. At five days post-transfection, media and cells were collected and processed for AAV purification. Cells were lysed in a high salt solution and treated with salt-activated nuclease. Media were PEG precipitated and resuspended in salt-activated nuclease solution. Both solutions were added to iodixanol density columns, ultracentrifuged, and extracted from the 40%/60% interface. Finally, AAVs were buffer exchanged, concentrated, titered, and (for vectors destined for non-human primates) assayed for endotoxin using Piece LAL chromogenic endotoxin kit (cat #A39552).

Retrogenix Cell Microarray

Retrogenix cell microarray was performed as described below for AAV analytes. Pre-screen optimizations were performed on slides of HEK293 cells and cells overexpressing mouse LY6A and human AAVR (KIAA0319L), TGFBR2, and EGFR. Transfection efficiencies were validated to exceed a minimum threshold prior to analyte application. AAVs were added to fixed cells at a concentration of 6×10⁴ AAV particles per HEK293 cell.

Biotinylated AAVs were created by incubation for 2 hours at room temperature with 10,000-fold molar ratio of NHS-PEG4-biotin (Thermo A39259) to AAV at 1×10¹³ viral genomes (v.g.) per mL in PBS. Reactions were quenched with 1 M Tris, pH 8 prior to buffer exchange, concentration, and AAV re-titer. Biotinylated AAVs were detected on HEK293 cells post fixation by AF647-labeled streptavidin. Unlabeled AAVs were detected on HEK293 cells post fixation by anti-AAV9 clone HL2372 (Merck, MABF2309-100UL) at a 1:500 dilution followed by AF647-labeled anti-mIgG H+L.

To achieve a suitable signal to noise ratio, necessary for minimizing false positives and false negatives, unlabeled AAVs were screened individually and as a pool at various concentrations, using anti-AAV9 detection. The final test pool was screened against fixed HEK293 cells/slides expressing approximately 6000 human plasma membrane proteins, secreted and cell surface tethered human secreted proteins and approximately 400 human heterodimers, each in duplicate. Hits were identified using ImageQuant as spots observed in duplicate. Following the screen, the 22 identified hits and CD86 positive control protein were spotted on new slides for individual AAV testing in a deconvolution screen. A negative control condition with no analyte and a positive control condition with CTLA4-Fc (to interact with CD86) were also included.

Protein Preparation

Lyophilized mouse LRP6 (AA20-1366) tagged with 6×His tag, N-terminal (E1E2) and C-terminal half (E3E4) fragments of mouse LRP6 (N-half: AA 20-628, C-half: AA 629-1244) tagged with Fc (mouse IgG2a), and full-length human LRP6 (AA 20-1368) tagged with Fc (human IgG₁), and LRP5 (AA1-1383) tagged with 6×His tag, and SOST protein were purchased from Bio-Techne (cat #2960-LR-025, 9950-LR-050, 9954-LR-050, 1505-LR-025, 7344-LR-025/CF, 1406-ST, respectively). Mesd protein was purchased from SinoBiological (cat #10949-H08H). All proteins were reconstituted in Dulbecco's phosphate buffered saline (DPBS, Gibco™) at desired concentrations before use.

Interleukin 3 protein from human (AA 1-152), mouse (AA1-166), marmoset, (AA1-143), and macaque (AA 1-144) were triple tagged with Fc-Myc-6×His, human and mouse GP2 (hGP2: AA 1-518, mGP2: AA 1-515) triple tagged with Fc-Myc-6×His, and human and mouse DKK3 (hDKK3: AA 1-350, mDKK3: AA 1-349) triple tagged with Fc-Myc-6×His were transfected into Expi293F™ (Thermo Fisher Scientific) cells at a density of 3×10⁶ viable cells/mL using ExpiFectamine™ (Thermo Fisher Scientific) according to the manufacturer's manual, and secreted proteins in media were harvested after 120 hours and cleared using a 0.45-μm PVDF vacuum filter (Sigma Millipore). Each His-tagged protein in media was captured with Ni-NTA resin (Qiagen) and eluted with DPBS containing 150 mM imidazole.

Human Adeno-Associated Virus Receptor (AAVR) PKD2 domain (AA 401-498) tagged with 6×His was purified using methods known in the art. Briefly, PKD2 was expressed in BL21(DE3)-RIPL E. coli. Cells were lysed by sonication, and the insoluble fraction was cleared by centrifugation. Cleared lysate was applied to a Ni-NTA column (Qiagen) and eluted using DPBS containing 250 mM imidazole.

Surface Plasmon Resonance (SPR)

A Sierra SPR-32 (Bruker) loaded with a protein A sensor chip was used. Fc-fusion proteins in HBS-P+ buffer (GE Healthcare) were immobilized at a capture level of 600-800 response units (RU) for FIG. 13B-FIG. 13C, FIG. 14D, FIG. 18A-FIG. 18B, and FIG. 20A-FIG. 20D, and 1200-1500 RU for FIG. 14B. AAVs were injected at a flow rate of 10 μL per min for 240 seconds followed by a 600 second dissociation. AAV concentrations began at 2.4×10¹² v.g. per mL and proceeded at 2-fold dilution intervals. A regeneration step with 10 mM glycine pH 1.5 was performed between each cycle. All kinetic data were double reference subtracted.

Pull-Down Assay

The pull-down assay was performed generally as described below. Briefly, prey AAVs were mixed with His-tagged bait protein and Ni-NTA resin in a binding buffer of DPBS containing 20 mM imidazole for 1 h at 4° C. on an orbital mixer. Resin was then collected in a spin column, washed twice with 10 column volumes of binding buffer and eluted in 45 μL of DPBS containing 150 mM imidazole. Eluate was analyzed by Western blot using anti-VP1/VP2/VP3 (ARP, cat #03-61058) and anti-6×His (Abcam, cat #ab18184) antibodies.

HEK293 Cell Culture Potency Assay

HEK293T cells in Dulbecco's Modified Eagle Medium (DMEM) containing 5% fetal bovine serum (FBS), 1% non-essential amino acids (NEAA), and 100 U per mL penicillin-streptomycin were cultured in 6-well plates at 37° C. in 5% CO₂. At 80% confluency, cells were transiently transfected with 2.53 μg plasmid DNA encoding a membrane protein hit from the Retrogenix cell microarray screen. Cells were transferred to 96-well plates at 20% confluency and maintained in FluoroBrite™ DMEM supplemented with 0.5% FBS, 1% NEAA, 100 U per mL penicillin-streptomycin, 1×GlutaMAX, and 15 μM HEPES. Plates were imaged 24 hours after application of AAV on a Keyence BZ-X700 (4× objective). For experiments with protein inhibitors, Mesd (26 ug/ml) and SOST (0.2 μg/ml) were added 4 hours prior to AAV addition. NucBlue™ Live ReadyProbes™ reagent (Hoechst 33342) was added to each well to aid autofocusing. Image quantification was performed using a custom Python image processing pipeline.

Primary Cell Culture Potency Assay

Human brain microvascular endothelial cells (ScienCell Research Laboratories, cat #1000) and cynomolgus monkey primary brain microvascular endothelial cells (CellBiologics, cat #MK-6023) were cultured as per the instructions provided by the vendor. The cell cultures were then treated with single-stranded AAV genome CAG-eGFP packaged viral vectors at a multiplicity of infection (MOI) of 5×10⁴ per well (4 wells per vector). The fluorescence expression of the culture was inspected and quantified one day after the infection procedure.

Animals

Adult (6-8 weeks old) homozygous B6;129S-Lrp6tm1.1Vari/J mice (Jackson Labs #026267) were retro-orbitally administered with 1×10¹² v.g. per animal of AAV1-X1 packaging either Ef1a-mCherry or Ef1A-Cre (N=6 per condition). After 3 weeks, those mice were re-administered with 1×10¹² v.g. per animal of PHP.eB or AAV-X1.1 packaging CAG-eGFP (N=3 per condition). Mice were randomly assigned to a particular AAV condition. Experimenters were not blinded for any of the experiments performed in this study.

Lrp6 Conditional Knockout Tissue Preparation and Imaging

Mice were anesthetized with Euthasol (pentobarbital sodium and phenytoin sodium solution, Virbac AH) and transcardially perfused with about 50 mL of 0.1 M PBS, pH 7.4 followed by an equal volume of 4% paraformaldehyde (PFA) in 0.1 M PBS. Collected organs were post-fixed in 4% PFA overnight at 4° C., washed, and stored in 0.1 M PBS with 0.05% sodium azide at 4° C. A Leica VT1200 vibratome was used to prepare 100 μm brain sections to be imaged on a Zeiss LSM 880 confocal microscope using a Plan-Apochromat 10×0.45 M27 (working distance, 2.0 mm) objective. Images were analyzed in Zen Black 2.3 SP1 (Zeiss) and ImageJ.

AlphaFold Structure Modeling

The complex structures of LRP6 ECD and AAV-X1 or AAV.CAP-Mac VR-VIII peptide were modeled using a cloud-based implementation of AlphaFold-Multimer-v3 provided in ColabFold v2.3.5. The input comprised two sequences: surface-exposed residues in VR-VIII of AAV-X1 (587-AQGNNTRSVAQAQTG-594, SEQ ID NO: 35) or AAV-CAP-Mac (587-AQLNTTKPIAQAQTG-594, SEQ ID NO: 36) and the extracellular domain of human LRP6 (UniProt entry 075581, residues 20-1370). The Google Colaboratory notebook was run using an A100 SXM4 40 GB GPU. Five structure models were produced using a protocol with up to 20 recycles, and MSA generated with MMseqs2 (UniRef+Environmental) and templates from PDB70. The structure models were ranked using a weighted combination of pTM and iPTM scores.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of increasing permeability of the blood brain barrier, comprising:
providing a targeting peptide capable of binding to low density lipoprotein receptor related protein 6 (LRP6), thereby increasing permeability of the blood brain barrier, wherein the targeting peptide binds YWTD domain 1 and/or domain 2 of LRP6.

2. (canceled)

3. The method of claim 1, wherein the permeability of the blood brain barrier is increased by at least 25%, 50%, 75%, 100%, or more as compared to the absence of the targeting peptide.

4. A method of delivering a payload to a nervous system of a subject, the method comprising:
providing a targeting peptide capable of binding to low density lipoprotein receptor related protein 6 (LRP6) or a derivative thereof, wherein the targeting peptide is part of a delivery system, and wherein the delivery system comprises the payload to be delivered to the nervous system; and
administering the delivery system to the subject.

5. The method of claim 4, wherein the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and any combination thereof; or
wherein the delivery system comprises a viral vector or a non-viral vector, wherein the targeting peptide enhances the binding affinity of the viral vector or the non-viral vector to LRP6.

6. (canceled)

7. (canceled)

8. The method of claim 5, wherein the viral vector comprises an AAV vector; wherein the targeting peptide is part of a capsid protein of an AAV vector.

9. The method of claim 8, wherein the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof.

10. The method of claim 5, wherein the non-viral vector comprises lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

11. The method of claim 4, wherein the payload to be delivered to a nervous system is a biological molecule, a non-biological molecule, or a combination thereof; wherein the biological molecule is selected from the group consisting of a nucleic acid sequence, a protein, a peptide, a lipid, a polysaccharide, and any combination thereof.

12. The method of claim 4, wherein the payload is a therapeutic molecule.

13. The method of claim 11, wherein the nucleic acid sequence to be delivered to a nervous system comprises one or more of:
a) a sequence encoding a trophic factor, a growth factor, or other soluble factors that might be released from the transduced cells and affect the survival or function of that cell and/or surrounding cells;
b) a DNA that restores protein function to humans or animals harboring a genetic mutation(s) in that gene;
c) a DNA that encodes a protein that can be used to control or alter the activity or state of a cell;
d) a DNA that encodes a protein or a nucleic acid used for assessing the state of a cell;
e) a DNA and/or associated guide RNA for performing genomic engineering;
f) a sequence for genome editing via homologous recombination;
g) a DNA sequence encoding a therapeutic RNA;
h) an shRNA or an artificial miRNA delivery system; or
i) a DNA sequence that influences the splicing of an endogenous gene.

14. The method of claim 4, wherein the LRP6 is a mouse LRP6; wherein the LRP6 has an amino acid sequence having at least 80% sequence identity to an amino acid sequence of SEQ ID NO: 29; or wherein the LRP6 is a macaque LRP6; wherein the LRP6 has an amino acid sequence having at least 80% sequence identity to an amino acid sequence of SEQ ID NO: 30; or wherein the LRP6 is a human LRP6; wherein the LRP6 has an amino acid sequence having at least 80% sequence identity to an amino acid sequence of SEQ ID NO: 31.

15. (canceled)

16. (canceled)

17. The method of claim 4, wherein upon binding the targeting peptide is capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

18. The method of claim 9, wherein the targeting peptide is inserted between two adjacent amino acids in AA587-594 of SEQ ID NO: 11 of the AAV9 vector or functional equivalents of AA587-594 in an amino acid sequence at least 80% identical to SEQ ID NO: 11; or wherein the targeting peptide is inserted between AA588-589 of SEQ ID NO: 11 of the AAV9 vector or functional equivalents of AA588-589 in an amino acid sequence at least 80% identical to SEQ ID NO: 11.

19. (canceled)

20. The method of claim 8, wherein the AAV vector is conjugated to a nanoparticle, a second molecule, or a combination thereof.

21. The method of claim 4, wherein the administration is a systemic administration.

22. The method of claim 4, wherein the subject is a human and wherein the subject is a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS; wherein the lysosomal storage disorder that involve cells within the CNS is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

23. (canceled)

24. The method of claim 4, wherein the subject is a subject suffering from, at risk to develop, or has suffered from a stroke, traumatic brain injury, epilepsy, or spinal cord injury.

25.-49. (canceled)

50. A method of designing a targeting peptide having specificity to low density lipoprotein receptor related protein 6 (LRP6), comprising:
generating in silico one or more targeting peptides each capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

51. The method of claim 50, wherein generating in silico the one or more targeting peptides comprises:
generating in silico a plurality of candidate peptides;
performing computer-assisted docking simulations for each of the plurality of candidate peptides binding to LRP6; and
analyzing the structure of LRP6 binding to one or more of the plurality of candidate peptides to identify one or more targeting peptides capable of interacting with (1) one or more positions functionally equivalent to R28, G158, E159, W183, A201, K202, or H226 in LRP6 having an amino acid sequence of SEQ ID NO: 31; or (2) one or more positions functionally equivalent to S96, S114, E115, R141, W157, W183, or W242 in LRP6 having an amino acid sequence of SEQ ID NO: 31.

52. The method of claim 50 comprising:
obtaining a binding score for each of the plurality of candidate peptides binding to LRP6; and
selecting one or more of the plurality of candidate peptides having a binding score above a threshold value as a targeting peptide having specificity to LRP6; wherein the method
